(19) United States
(12) Patent Application Publication
(10) Pub. No.: US 2005/0130125 A1
(43) Pub. Date: Jun. 16, 2005

(54) End of aids for general virology, based on profound science as protein foldings: safe vaccines, universal antimicrobial means, mad cow end

(76) Inventor: Yuly Zagyansky, Paris (FR)

Correspondence Address: Y. Zagyansky, Entraide, 22 rue Ste Marthe, Paris, 75010, FR

(21) Appl. No.: 10/505,353

(22) PCT Filed: Mar. 4, 2002
(86) PCT No.: PCT/EP02/02302

(51) Int. Cl. C12Q 1/70

(52) U.S. Cl. 435/5

(57) ABSTRACT

All AIDS principal mysteries are resolved. “One step” AIDS is successive contaminations.(with low [anti-env]). Strong sole lethal animal doses are confirming. Mobility macrophage (mφ) receptors contaminate at 1st stage with nonproductive entry with nonintegrated and heterogenous (due to nef) HIV DNA and proteins and preudoinfectious A particles. Such heterogeneity is obligatory for 2nd productive contamination with heterogenous anti-env, with integrated DNA and homologous proteins. Encephalites are due to moving into brain myp due to locally liberated cyto and chemokines Nongenetic factors are determining: at general persistent seronegativity (contact regularity) or absence of 2nd contamination due to different Fc receptor carbohydrates (babies before 3 months, chimpanzee). Minor genetic factors (as CCR5-2) only modify. AIDS in explaining all dangerous vaccinations. Carbohydrate origin of NK-cell mechanism. Artificial. virus culture contaminations. HIV signallings are resolved with general laws of functional recognitions and foldings with help of Universalest “Du-2T”-like peptides and 2 prolyl-isomerases (coupled trans-cis transitions). Chaperons protect proteins against intercarbohydrate aggregations. Prione “scarpie” state is artificial dissociation of their “Du-2T”. MHC and TRC allbtypes are due to their carbohydrates. Hearts of any cell functionning: “PKC” transporting vesicle cycle and independent direct DNA activation. Apoptosis: irreversibility of preparation of next signal with stock exhaustions. Ribosome cycles. Key proprotein primary structures confirm above data. Consequences of profoundest bases: mφ mobility stopping against encephalites; charged antibodies eliminate viruses, cancer cells and harmful antibody clones (anti-viral ,anti-auto); “Du-2T” eliminates viruses and “Mad Cow”; vaccines from homogenous viruses with one neutralizing epitope and correct virus titres; ribosomal protein synthesis; means against clinical death and coma; perfectest hypnotics.

### TECHNICAL FIELD OF INNOVATION

Innovation concerns the solutions of principal “mysteries” of AIDS, generalized with the fundamental problems of General Virology, based on the fundamental science as the resolved general mechanism of the protein foldings, functionings and recognitions and their practical consequences. non-dangerous immunisation, Universal antimicrobial preparations, determinations (in 1st time) of the correct viral concentrations, preparations against encephalitis, Mad Cow end, correct effective protein synthesis in vitro, creation of correct enzymes (proteins).

Table of contents.

| | |
|---|---|
| Specification | 1 |
| Technical field of invention | 1 |
| Table of contents | 1 |
| Summary | 3 |
| Text of Specification | 10 |
| Part I. AIDS: to the end. 1st Entry of HIV is due to Cell Movement | 10 |
| and Cell Signalisation | |
| §1. Cell motility is responsable for 1st virus entry | 13 |
| §2. Cytokines as intermediates during signalisations after HIV infections | 14 |
| §3. CD4- envelope interactions. Absolutely new molecular bases of their | 15 |
| interactions. General Universal Law of Switching of the surface receptors | |
| §4. Cytokine receptors are responsable for 1st entry of HIV viruses | 16 |
| into the monocytes/macrophages during their movement | |
| Part II. AIDS: to the end. Molecular Bases of HIV Interaction with Target | 18 |
| Cells and General Scheme of AIDS Development | |
| §1. Proofs of delayed anti-env antobody production with help of the new | 18 |
| law of NK- cell selections | |
| §2. Intercarbohydrate mechanism of gp120-CD4 interactions | 21 |
| §3. Basis of HIV-CD4 interactions: new law of intercarbohydrate | 23 |
| homologous interactions | |
| Appendix | 24 |
| Part III. AIDS: to the End. Two Special Pathways of intracellular HIV Development | 25 |
| in Vivo and their Important Consequences | |
| §1. Molecular proofs of two types of HIV entry. Intracellular A-particles are clear | 25 |
| reason of false viral presences after 1st contamination (seropositives) | |
| §2. Confirmations of mechanisms of HIV behavior with zidovudine (AZT) | 28 |
| §3. Intracysternal A particles are the artefacts of HIV presence in seropositives | 29 |
| §4. The heterogeneity, immunosuppression and the net function | 29 |
| §5. Clear nongenetic origin of persistent seronegativity | 31 |
| Part IV. AIDS: to the end. Further Confirmations of General Strategy and | 33 |
| Molecular Characteristics of AIDS Development | |
| §1. Mysteries of epidemiology well fit the AIDS strategy | 33 |
| §2. Development of AIDS course by insects only at 2nd functional contamination | 33 |
| during certain blood insect mechanical transmission of any virus | |
| §3. Opportunist infections: facilitation of 2nd contamination at AIDS | 35 |
| stage by complement receptor due to activation of C1q fragment | |
| §4. “Immunity” of chimpanzees as weak correspondence between Fc chimpanzee | 35 |
| cell machinery and HIV-1 pattern of carbohydrates | |
| §5. Child's AIDS adapted to particular characteristics: active immune system, | 36 |
| mother's antibodies and change of Fc receptor carbohydrate pattern around | |
| 3 months of age | |
| §6. Principal schema of HIV infection (in vivo) | 38 |
| Part V. AIDS: to the End. Clarification of Vaccinations: Solutions and Impasses: | 39 |
| Generalisations for Virology and Simply Makable Experimental Confirmations | |
| §1. Clear principal utopy of actual strategies of vaccinations against HIV. | 39 |
| Charged anti-envelope (env) antibodies and specific elimination of clones of | |
| anti-env antibodies as effective means against AIDS | |
| §2. Artificial conditions of HIV contaminations in vitro | 41 |
| §3. Unnaturally large dozes lead to dispensibility of 2nd virus introduction. | 42 |
| Confirmation of general strategy. | |
| §4. Lower variability of 1st stage and worse fit of the carbohydrate pattern | 42 |
| must be responsable for restricted HIV-2 contaminations | |
| §5. Generalisations for Virology | 43 |
| §6. Clearly and simply makable definitive experimental conditions. “Troyan | 45 |
| man model” | |
| Part VI. Practical consequences of Parts I-V | 46 |
| Part VII. Chaperons: Principal Solution of Functioning of Protein Folding. | 48 |
| Universal Recognition Mechanisms | |
| §1. Endoplasmic reticulum→Golgi secretory pathway | 49 |
| §2. Cytoplasmic chaperon machinery | 53 |
| §3. Different types of Protein Foldings- The Fundamentalest Bases of | 54 |
| their Functionings and Recognitions. | |
| Part VIII. IgG Priones as Clear Example of the most Studied Case | 56 |
| Part IX. Machanism of Switching of Cytolysis with Help of MHC Class I- Peptide Interaction | 57 |
| with T Cell Receptor as Consequence of General Chaperon make up | |
| Part X. Net Solution of the Process of Formation of Priones: Primary Cause of Mad | 60 |
| Cow and Creutzfeldt-Jacob diseases is Artificial Dissociation of “Du-2T” peptides | |
| ANNEX. Part IA. Universal Propagation of Signal from Plasma Membrane to | 61 |
| Nucleus. Increase of pH→Increased Synthesis of Cathepsin L (CL)→Liberation of | |
| CL Stocked on Ribosomes→Liberation of Nuclear Factors→Activation of Genes | |
| §1. Na⁺/H⁺ exchanger activation with rapid cytosolic diffusional (all over) | 62 |
| pH increase. Increased protein synthesis | |
| §2. N-methylation of proteins and protein-nucleic acid interaction | 62 |
| §3. Activation of Cathepsin L | 64 |
| §4. Activation of alkaline phosphatase (AP) | 65 |
| §5. Limited proteolysis of Nuclear Factors and nuclear signal | 65 |
| §6. The CL activates the ribosomes in the cytoplasm. Ribosomal tournover | 66 |
| §7. General position of such signal: Plasma Membrane→Nucleus in cell signaling | 67 |
| Part IIA. Primary Nucleotide Structures of Important Proteins and Mechanism of Process | 68 |
| of the Special Universal Transduction of the Signal from Plasma | |
| Membrane to Nucleus | |
| §1. Presence of Gly-Arg sequences in other signaling proteins of “PKC” vesicle | 69 |
| machinery and their isoforms | |
| §2. Stop codons stabilize stocking | 73 |
| §3. The mRNA as guide for its ribosomes | 74 |
| Part XI. Practical consequences of Parts VII-X and Annex | 74 |
| Part IIIA. Perfect Proof of Existence of Transporting Vesicle Cycle with Muscle Physiology | 77 |
| References | |
| Preliminary International Classification of Patents and Legality of Patent Strategies | 87 |
| Legends to Figures | 88 |
| Claims | 91 |
| Figures | 1/3 |

### SUMMARY

I. It is proved that it is only the moving macrophages that are contaminated during their movement during first of two necessary contarminations for AIDS development. This movement is initiated, in particular, by secretion of cytokines that induct an appearance of the corresponding adhesion molecules in target organs. Also a number of β-chemokines are released by the infected (by HIV) macrophages after the release by them of special chemokines serving as chemoattractants for directed macrophage movement. And justly, these β-chemokines with corresponding β-chemokine receptors coinfect the cells (macrophages/monocytes) with HIV. An absence of AIDS at men with the inactive β-chemokine receptors (CCR5-2) is genetical leading to absence of 1st contamination without subsequent antibody appearance and AIDS stage. Relatively low concentrations of HIV can make the 1st contamination in vivo due to justly the cell movement with patching, capping and endocytosis also of many receptors participating in cell motility in difference with “classical” endocytosis of one kind of receptors with relatively high threshold for receptor-ligand concentrations. Justly the common aggregation of different receptors (with created glycoprotein-sphingolipid complexes) provokes quicker the common aggregation of syndecans, whose “critic mass” makes the microfilament attachment with corresponding signal machinery that switches the ATP-dependent aggregation of patches (capping) and endocytosis. A number of cytokines are the Intermediates during the signallings leading to the migrations and adhesions of contaminated macrophages with corresponding viral protein synthesis (at 1st contamination) and during the permanent productive virus activation with help of Fc receptors (at 2nd contamination). The discrete signal for switching of CD4 and HIV env molecules during their interaction, leading to creation of the signal receptor-glycosphingolipids agreggates, is done with dissociation of Universal little proteins (“Du-2T”) (nearby hinge, normally), switching a liberaction of carbohydrate chains for new interactions (Parts VII-X).

II. An evidence of the tard appearance of the anti-envelope (HIV) antibodies due to a presence of the NK- cells, preferably against the env proteins, is presented. The established here mechanism (with absolutely new principle) of the NK- cell selection due to the intercarbohydrate homologous interactions between NK and virus contaminated cells helped to resolve this problem. According to principal scheme of interactions between CD4 and viral envelope gpl2o molecules there is an establishment of the fixed new homologous intercarbohydrate interactions between O-chains of CD4 (near sensible hinge region) and V3 region of gp120 instead of similar interactions between O-chains of C2 and V3 regions of gp120 and homologous ones of the CD4. Proposed molecular bases of the fundamental law of the intercarbohydrate homologous interactions prove that justly the spread (In vivo) carbohydrates (as glucose, N-acetylglucosamine, α-sialic acid, β-galactose, N-acetytgalactosamine) were selected to participate in such interactions A number of cases with fit interactions of homologous oligosacharide chains are found already: synthesis of cell surface carbohydrates during cell motility, aggregation of glycosaminoglycans, IgG (Fc) attached oligochains, IgA and IgD O-chains, distal saccharides of gangliosides and glycoproteins, carbohydrate chains Le^{X}. A correct finding of numerous homologous interacting oligochains is sufficiently routine work today.

III. The nonproductive ist HIV entry takes place during the macrophage cell movement with endocytosis, with unintegrated replicated viral DNAs, with heterogeneity (although limited) of DNAs and corresponding proteins, with presence of intracystemal A-particles (tAP) and with the pattern of antibodies against the corresponding heterogeneous viral proteins. Such lAPs, justly, make the very weak virus pseudocontamination of other cells due to RNA and reverse transcriptase that they contain. The gag coat of such preudoparticles is assembled in cytoplasrne and their RNAs are not transported with ribosomes to the plasma membrane. All principal “contradictable” zidovudine (AZT) experiments are well explained by such contaminations strategy of AIDS development. The heterogeneity of env viral proteins is generally absolutely necessary for later productive HIV contamination due to a number of the aggregated Fc receptors with Fc fragments of antiviral (often anti-carbohydrate) antibodies provoking the corresponding signal. The nef protein is indispensable for such necessary heterogeneity after the DNA reverse transcription. Also another nef protein function is an attenuation of the cellular machinery activity that, logically, must permit a better presentation of endogenous secretable viral non-env molecules to MHC-II molecules. Justly such autoantibody population after above nonproductive 1st viral entry is responsable for persistent seronegativity. Evidently, the persistent seronegativity after regular contacts with AIDS sick patients is not of genetical origin as well logically the lower seroconversion of partners without condom use at all or of borrowers of injecting equipment. A longer survival due to particular MHC alleles /allospecificity of which is determined (firstly) by their specific carbohydrate pattem/ had not to be the dominating factor (as well due to particular TAP alleles). Moreover the cellular cytotoxicity (in AIDS case) is very uneffective de facto.

IV. A convergence of interpretations of different facts, that confirm the AIDS development strategy, is striking. Even statistical epidemiological data (drug users, sex histories ,transfusions) argue for the 2nd contamination. Although sometimes, the Immediate 2nd contamination (with low level of antibodies) is not seen and patients, already progressing to AIDS, are mistakenly considered as simple seropositives. An improved sterility of blood transfusions or long monastery life diminishes (elininates) a number of 2nd contaminations and sharply increases a median survival. A sure transmission of contaminated blood (evidently, without exception for HIV) by insects, with progression to AIDS only after 2nd weak doze contamination, explains clearly an absence of AIDS at old men and normal children wivth well diminished relation of numbers of seropositives and AIDS-sick in insect dominated area. A presence of Insect pruritic papular eruptions as initial manifestation of AIDS stage happens in insect dominated areas. An acceleration of death at AIDS stage could take place due to facilitation of contaminations at this stage by complement receptor (CtqR) with Ciq factor, activated by antibodies against opportunist agent. A bad correspondence between Fc receptor chimpanzee cell machinery and HIV carbohydrate patterns (logically) prevents the 2nd contamination and AIDS phase (“immunity” of chimpanzee). In very complex case of Infant AIDS,. the general HIV strtegy and new molecular characteristics confirmingly well coincide. Infants macrophages are more active than adult ones and the infant immune system can already produce antibodies very soon after birth (including anti-HIV). But 2nd AIDS stage can happen only from ˜3 months of age due to created correspondence of carbohydrate patterns between Fc receptor machinery of infant macrophages and HIV virus. Generally, the 2nd contamination could be done only with contaminated milk. The long term infant AIDS must be due to very small dozes transmitted by parasites or due to bad hygiene because of AIDS-sick nearby parents. The macrophage tropic clones have the best (corresponding to macrophages) carbohydrate pattern and contaminate them at 1st contamination. At the 2nd contamination, also the macrophage tropic clones contaminate, with help of antibodies already, the macrophages and (in smaller degree) lymphocytes T4. But principally, only the macrophages produce in vivo, by budding, the new viral particles, but the T-cells undergo the apoptosis with syncytium creation. The macrophages well phagocytize (regularly) cells during apoptosis, so the contaminated T4 cells are destroyed until host death by relatively small groups that are not well visible.

V. The development of AIDS mechanism permits even the great revolutions. Because antibodies help to make the productive HIV infection, one can wait that the vaccination aggravates the contamination. But due to vaccination with homogenous envelope proteins, the strong productive contamination is problematic, and these homogenous antibodies some diminish a quantity of virus particles (precipitation) and also encumber (with some delay only) the. 1st entry with heterogenous proteins production with help of the macrophage motility although even homologous antibodies can (at some special conditions) make sporadic productive (2nd type) entries into cell. So such vaccination strategies are not acceptable. The signaling specific perturbation from outside with charged antiviral antibodies or an elimination of the anti-envelope antibodies clones with, also charged, idiotypic antibodies (against anti-envelope antibodies) must be successful. Two necessary types of HIV consecutive contaminations in vivo must also take place during only strong artificial contamination where anti-envelope antibodies meet the yet noneliminated introduced active viral particles, confirming the general strategy. The HIV-2 restricted contaminations are due to weaker variability of viral proteins at 1st contamination and larger differences between host and virus carbohydrate patterns. Generalizations for other viruses are evident. The 1st nonproductive contamination, also with utilization of cell motility, must be, for exemple, used by herpes viruses and the antibody-dependent enhancement is a property of viruses of a number of Families. The profound properties of HIV lentiviruses must be much more general and even could be revolutionary In all virology. For instance, ununderstandable variabilities. of viral vaccinations since LPasteur (until grave effects) must be a consequence of homogeneity or heterogeneity of antiviral antibodies, so all viral vaccines and antiserums must be urgently reviewed. These data confirm that nowdays contaminations in vitro take place due to increased virus concentrations with increased number ofiercarbhydrate homologous interactions and meltings with fusions and clearly do not correspond to the both types of contaminations In vivo.

VI. The above characteristics of HIV with their Generalisations for All Virology (as contamination during 1st phase by relatively weak virus concentrations by mobile macrophages, the indispensable heterogeneity of viral proteins, synthesized during 1st phase to execute 2nd contamination, the new type of viral productive contamination with anti-viral antibodies during 2nd phase of disease) permit make an application for ALL Virology. One can estimate the grave unpredictable effects of vaccinations (since Pasteur and this is Revolution) against such spread virus as Influenza A of influenza/pneumonia in immunizing with viral surface glycoproteins with one sole neutralizing epitope, obtained from one homogenous strain. In perturbing the macrophage motility, resolved (Y.Z. ISBN:2-9502914-8-1) (for instance with antibodies against specific receptors of macrophages as chemokines-f), one prevents the encephalites of different viruses. In approaching the conditions of determination of virus titers in vitro to ones in vivo (as gradient of concentrations of chemokines for 1st contamination or heterogenous anti-env antibodies for 2nd one), one will obtain real viral concentrations (in vivo) in first time.

VII-X. The resolutions of principal “mysteries” of AIDS are based on fundamental science as protein foldlngs. The intriguing folding of proteins with help of chaperons is resolved. There is a specialization of chaperons for each type of glycosylation: N-, O- and GAG-, that determines the Universal specialities of the limited number of chaperons for enormous number of proteins. The function of chaperons is to protect the proteins from their aggregation by their homologous carbohydrate chains. There are 2 principal pathways of the protein foldings: endoplasmic reticulum (ER)→Golgi and in cytoplasm. There are 2 types of “Boats” of corresponding different complexes. The active state of all proteins with necessary tension for stock of energy during protein functioning is made with 2 enzymes peptidyl-prolyl isomerase (PPI) making 2 coupled transitions (prolyl trans-cis of two prolyl neighbour residues with weak energy differences between these positions) where the return is impossible because the sole transition of return is impossible and two transitions (at the same time) are unprobable. In the case of the pathway ER→Golgi, there is the protein folding with OBLIGATORY help of the coupled propeptide (from terminals N- or C-), attached to the “host” macromolecules, situated near 2 prolines (trans→cis) and hidden by 2 homologous interacting carbohydrate chains, permitting to create, “at once and for all time”, a possibility to stock the tensional energy (“folding state”)/with help of PPI and protein-disulfide isomerase (PDI)/, where the dissociation of this peptide (Little Protein- “Du-2T”- like), these times, only changes the state of general structure in liberating the carbohydrate chains for, already, INTERmolecular interactions. A real existence of these Du-2T- like proteins is proved in the case of IgG, Fc receptors and receptors for antigen and also for MHC molecules class I. The detailed mechanism of interactions between MHC-I molecules and T cell receptor (TCR) is resolved (with “Du-2T”s, concrete carbohydrate chain interactions, special /justly!/ destabilizing charges in intramembranous domains of all components of TCR). The VERY important allotype specificity between TCR and MHC molecules is determined by common ologocarbohydrates. In the case of folding of the cytoplasmic molecules of the “PKC” (Protein Viinase C) vesicle machinery (steroid receptors or bFGF included) that have the “propeptides with GlyArg”, there is the famous “mysterious” “trips” of these yet unfolded molecules to preproribosomes in nucleus, their return in cytoplasm (attached to proribosome), the cutting (with signal) of this “GR propeptide” (that is “becoming” “Du-2T”) by Cathepsin L (CL), an attachment of this “Du-2T” to “host” protein and consecutive folding of this protein with chaperons, also attached to.ribosome. A dissociation of “Du-2T” from “host” protein is stimulated by specific carbohydrates of target (“adress”!) Interacting with both carbohydrate chains, that hide the “Du-2T”. In the case of “pure” cytoplasmic proteins (representing many enzymes) (without “trip” to nucleus), the folding is necessary for activity (like enzymatic for stock of tensional energy) where this “propeptide” “Du-2T” (evidently, normally small proteolysed part) is necessary for correct folding with help of PPI and where its dissociation must conduct to the dissociation between homologous carbohydrate chains to interact specifically with chains of target (“adress”!). In the case of proteins of pathway ER→Golgi, the artificial lost of these “Du-2T” conduct to the pathology: creation of aggregates of these proteins by their carbohydrate chains (so called “scarpie”) like during diseases: Mad Cow or Creutzfeldt-Jacob. Also the dissociation of these proteins “Du-2T” is the partie of action of parasites: like viruses, bacteria, protozoans, mushrooms, during complement action, cell lysis, aggregation of proteins in solutions (as blood). So even a simple introduction of these “Du-2T” eliminates these grave processes. It is proved that Erp61 is not the chaperon. but the vesicular Phospholipase C-α.

ANNEX (basic foundation of VII-X). IA. Universal propagation of signal from Plasma Membrane to Nucleus: increase of pH→increased synthesis of Cathepsin L (CL)→liberation of CL stocked on ribosomesr+liberation of nuclear factors→activation of genes . . . New Universal process of primary activation of DNA during beginning of the signal from cell surface is proved definitively. Such signal propagation contains an activation of echanger Na⁺/H⁺ followed by increase of intracellular pH. In difference with the. typicaltransport of substances par vectorial transport with vesicles-carriers, these ions H⁺ and Na⁺, really (as it was accepted until today for different molecules, Ca²⁺ and phosphatidylinositol phosphate derivatives) propagate by diffusion until nucleus. . This pH increase induces an increase of protein synthesis on ribosomes, CLs included, making the part of them free from rRNA attachment in cytoplasm (previous stock). Such free CL make autoactivation with activation of ribosomes by limited proteolysis of particular proteins of ribosomes and liberate (par limited proteolysis) other proteins (of signal machinery) also attached to rRNA with help of asymmetric dimethylarginine in sequences GR at protein, extremities. The alkaline phosphatase helps to such hydrolysis In eliminating the protective phosphorylation by casein kinase II (CK-II). Such detached molecules help to reconstruct (without any transcription, translation and transport of proteins) the vesicular obligatory vectorial transport machinery (heart of each complete cell signal). The nuclear factors, detached at the same time (by CL), activate the immediate-early genes, provoking the following chain of genes activations. The stock complex Rel: p105(p50)/p65(RelA)/IkB-α waits the signal in cytoplasm,. being activated, firstly, by CL. The stock complex Rel: p1o5(p5o)lp65(ReIA)/lIkB-:a waits the signal in cytoplasm, being activated, firstly, by CL Other signal events as a creation of the special network of proteins in plasma membrane or the integrin interactions serve, mainly, for creation of the exterior parts of the vesicular transport machinery. The primary. origins of apoptose is the irreversible impossibility to make the beginning of the next signal because of an absence of the stocked machinery proteins and/or the exhaustion of molecules of phosphatidylinositol 4,5-bisphosphate. The intriguing cycle of ribosomes /noyau (preproribosomes)→cytoplasm (preribosomes)→cytoplasm (active)/with signals is well outlined.

IIA. Primary Nucleotide Structures of important proteins and the mechanism of process of the special Universal transduction of the signal from plasma membrane to nucleus. The serious proves for clear reality of the stocking of different important proteins are found in convergence with a number of their primary nucleotide sequences. An insitent presence of sequences GR at extremities of these proteins and their absence at other parts show the sites of their attachment to rRNA in ribosomes (with help of specific asymroertic dimethylarginine) and the action sites of CL. A correlation of an activation of CL, c-jun, p53, PKC (all principal forms!), phosphatidylinositol-specific phospholipase C (PI-PLC) (all principal forms), α-subunits of vesicular CK-II), bFGF, steroid receptors, complexes Rel with their (observed already) limited proteolysis, confirm this functional pathway of these molecules. In the case of PI-PLC, the limited ACTIVATING proteolysis by CL must even. produce the Interacting subunits. Exceptionally, the interior unproteolysable regions of the actins and bFGF, with sequence GR, correlate with their exceptional protections with Mg ions and heparin sulfate correspondingly. The persistent presence of stop.codons upstream of 5′-end of open reading frame stabilizes the mRNA, that, justly, coincides with prolongated time of stocking between signals in G0 or after phase G1. The increased. rate of a metabolism for nonstocked forms of some signalling proteins correlates with particular compartirientalizations (as growth cone or cell periphery) of their corresponding ribosomes directed by particular modifications of their present mRNA!

Part XI .From general Universal proof of protein Foldings, functionings and recognitions and consequently the action of viruses (and also bacteria, protozoans and machrooms) with help.of Little Protein (Du-2T- like peptides), one can use them as Universal means against such parasites. Also being a cause of diseases as Mad Cow or Creutzfeldt-Jacob (due to their/“Du-2T”/artificial dissociation frorn particular functional proteins synthesized in ER-Golgi), one can stop such diseases with these corresponding “Du-2T”. A creation of new enzymes (proteins) must be done in accord with general Rules of foldings and the effective protein synthesis in vitro (on ribosomes) must be done with cytoplasmic ribosomes, activated with CL. Due to established molecular origin of the apoptosis (end of the stock of the proteins of the transport cell machinery and the hydrolysis of the phosphatidylinositol 4,5-bisphsphate /PIP₂/ par functional /that was proved here/ PI-PLC-α/vesicular/), one can utilise the derivatives of PIP₂ against state of clinical death and coma. Also in accepting that the sleep is the partial reversible apoptosis of cells of the system of cyclic neurons in superior brain (determining conscience), one can utilize the substances that partially disrupt the vesicle transport system of such neurons as cyanates as hypnotics.

IIIA. Perfect proof of existence of transporting vesicle cycle with muscle physiology.

The clear “PKC”-like transporting vesicle machinery cycle (moreover permanent) is well visible in skeletal and cardiac muscles. Each cycle must correspond to small sarcomer displacement. And justly In order to make completely the whole sarcomer displacement, the “train” (always) of nerve action potential arrives to muscle (fast fibers in skeletal muscles). Moreover, the “strange” slow fibers with strange “chaotic” innervations (always present between fast ones) enable to keep the contraction level between the action potential “pauses” in “trains”. So the proofs of such excellent permanent functional transporting vesicle cycle in muscles are very Impressive.

### TEXT OF SPECIFICATION

Parts I-VI of this Innovation were already done in 1998 (Patent Application FR-98-03204, date of filing 17/03/98, retired) and this PCT Application is the translation of this text (pp.1-97) with some reductions because of my situation (without the clearly resolved mechanism of insistent appearance of tumors at the real AIDS phase, pp.46-48, claims 1 and 7), that could be done only with profoundly resolved principal mechanisms of cell cycle and cancer (FR-95-11550, pp.1-77, retired). To make the fundamenatal proofs of HIV virus envelope- CD4 receptors interactions (where a large number of the needed corresponding data were missed), I had to resolve the UNIVERSAL GENERAL mechanism of protein foldings, functionings and recognitions (FR-99-12670) (translated, in Parts VII-XI and Annex) where the basic VERY fundamental Annex data make such Universal mechanism as profoundly proved.

Part I: “AIDS: to the End. The 1st Entry of HIV Due to Cell Movement and Cell Signalisation”.

12 years ago (1990!), I resolved already the main problems concerning the bases of AIDS /t/ showing the OBJECTIVE, relatively simple, way of the definitive EEXPERIMENTS. Visibly, because this detailed publication was done by Industrial Property (1990) arnd was not widely known (see for Instance an analogy of similar publication /2/ with accompanying scientific publications), it did not influence the research where justly at 1989-1990, the situation was the best to resolve the “special” mysteries of AIDS. So firstly I must resume the main, yet too advanced, characteristics of the above globally correct Inventions, absolutely necesaiy for understanding, with new illustrations which are clearly valid for present inventions too, where I strongly confirm, well develop and precise the discoveries.

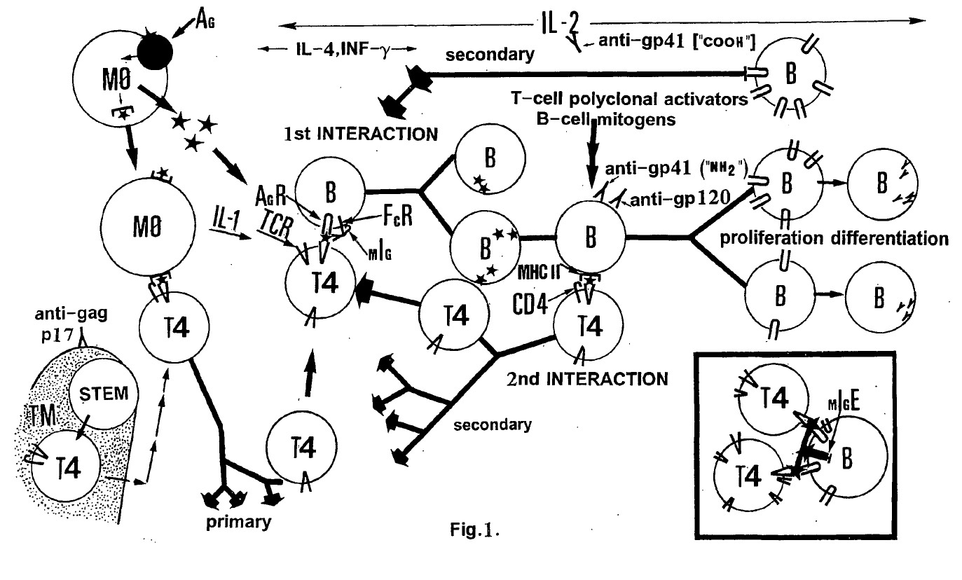

In the beginning, I resume the new, developed by me, general immunology /1/ without which I could not resolve the whole problem. Moreover, a perfect resolution of such complex AIDS problem confirms a validity of this new immunology. Principally, there are two consecutive physical interactions between lymphocytes B and T4 (FIG. 1). The 1st one takes place without interaction of CD4 (T4 cells) with MHC-II (B-cells) but with help of membranous immunoglobulins (Fc-receptor) and B-cell antigen receptors. And 2nd one takes place with help of the CD4 and MHC-II interactions already leading to the antibody production. The T-cell polyclonal activators and B-cell mitogens can act only (justly) at 2nd stage.

TABLE 1
Spectrum of the subclasses and classes of immunoglobulins serves to
determine a sequence of events after the HIV contamination in vivo /1/.

| protein | observation | explanation |
|---|---|---|
| gag (pol) | increased quantity of | repetitions of the antigen |
| | IgG4, IgE (IgG2, IgG3) | introduction |
| gag (pol) | increased quantity of | repetitions of the antigen |
| | IgG4, IgE- hemophiliacs | introduction |
| gag p55 | often IgG4 subclass | synthesis of p55 de novo with |
| (unproteolysed) | | several antibody response |
| | | inductions before anti-env |
| | | antibody synthesis |
| env gp41 | only subclass IgG1 | the latest appearance |
| env gp120 | only subclass IgG1 | the latest appearance |
| env gp41 | there is no IgM | 1. antibodies are autoimmune |
| env gp120 | there is no IgM | 2. “short-circuit” [symbol] |

[symbol]

“Short-circuit” is the activation, proliferation and differentiation of the B- cells by anti-gp41 and anti-gp120 (anti-MHC-II) antibodies instead of the T- cells in 2nd meeting. During “short-circuit” there are: 1) rapid inclusion of the memory B- cells; 2) absence of the 2nd search between B and T cells; 3) polyclonal force.

At 1st contamination there is a penetration of viral particles into the differentiated macrophages where, as result, a majority of these macrophages produces noninfectious viral proteins serving for antibody production. Unexpectedly, de facto, these antibodies are created in different periods where the anti-env (antiviral envelope) antibodies are created later /during ist(s) GLOBAL contamination(s)/. I concluded it from the analysis (Table 1), because (principally) In the case of the anti-env there is a presence of only the “early” switching subclasse IgGl. It means the IgGi anti-env appears only one time being also the last one, but in the case of anti-gag (viral proteins) there is a presence of the later appearing subclassses reflecting previous statistical numerous viral contaminations where the threshold for the necessary quantity of original viral particles for an appearance of the anti-gag is lower than for the anti-env particular contamination.

And this is very principal point. During natural contaminations, the infectious viral particles are eliminated from the blood. strongly before an appearance of anti-env antibodies. Justly, this situation permits the virus to develop the principally new interactions: a beginning of infectious viral particles production with help of the anti-env antibodies and Fc receptors (FcR) during the next new contamination.

The molecular bases of the PGL (persistently generalized lymphadenopathy) (persistently seronegatives) (Table 2) are much more difficult case that could be

TABLE 2
The truth global classification of the clinical cases of AIDS
according to the established molecular basis [symbol] /1/.

| | clinical case | molecular basis |
|---|---|---|
| Group N^{o}1 | “simple” | contact(s) with (at the end) |
| | seropositives | seroconversion to anti-env, no |
| | | progression to AIDS |
| Group N^{o}2 | imminent AIDS | new contamination(s) after |
| | high level | appearance of anti-env antibodies |
| | of anti-env | |
| Group N^{o}3 | persistent | consequent contact(s) with virus before |
| | seronegativity | before appearance of anti-env/ |
| | no anti-env | immunosuppression with anti-gag p17 |
| | antibodies | (anti- - Tα)/, “autoimmunisation” |
| | | against AIDS by total suppression |
| | | of anti-env production |
| Groupe N^{o}4 | imminent AIDS, | anti-env production is appeared in |
| | low level of | spite of suppressions during consequent |
| | anti-env | contamination(s) before the anti-env |
| | | production |

[symbol]

Evidently, the CD4- cell count or the tests as anergy etc in the beginning of AIDS stage (Groups N^{o}N^{o}2 and 4) must distinguish these Groups from Group N^{o}1.

resolved only with the created correct immunology. It was concluded that an appearance of the antiviral antibodies takes place with help of the auto-anti-MHC II antibodies (anti-gp4i and gpi2o env proteins) triggering the polyclonal actvation of B-cells instead of the 2nd phase of B-T interactions (FIG. 1, Table 1) (“short circuit”). These antibodies justly have different properties: polyclonality, absence of complement activation, their production without distinguishing between the MHC-III alleles. If the 2nd contamination takes place during the suppression of the hormone thynosin -α (Tα) (important for T-cell development) with developing of the auto-anti-p17 (gag) (it means: anti-Tα) antibodies (making suppression of the development of the T4 cells), but before an appearance of the anti-env, such suppression could lead to a total suppression of the anti-env production and an absence of (even) the “short circuit” help. This means: to the permanent absence of AIDS. The truth global classification of clinical cases of AIDS is given in Table 2.

The Insects cannot induce a seroconversion (1st contamination). But they can transmit a smaller quantity of particles sufficient for 2nd contamination /1/. These conclusions clearly explain an absence of AIDS at children (nonvertical transmission) and at eldery as well an increased quantity of the AIDS cases inr regions with a lot of insects and an appearance of the pruritic eruptions resembling the bite of an insect in a middle on the open parts of body (only in South region!) which are initial signes and symptoms of AIDS. Justly a presence of virus in the mother's milk (regularly consumed) must conduct to the development of AIDS.

§1.Cell Motility is Responsable for 1st Virus Entry.

The most important concluded feature was the two steps of the AIDS development with 2 corresponding contaminations /1/. But why these consecutive contaminations are different? it happens because there is already a presence of anti-env antibodies after 1st effective contamination /1/. Moreover, such meeting between the virus particles and the anti-nv antibodies had not to be happen before due to the destruction of the infectious viral particles before a late appearance of justly anti-env antibodies. In reality, it is -not an increase of the infectious viral- production, but the principally different form of the virus (viral. proteins) contamination takes place at 2nd contarnination. And the very solid chain of the molecular level data proves that during the 1st contamination there is no production of the Infectious particles but during the 2nd one, this production takes place. In particular, the entry of the virus with help of the cell motility takes place only at 1st entry.

It is found that at the 1st stage, only a small quantity of the monocytes is contaminated /1/. Logically, it must be the motile monocytes because the de novo expression of the special adhesion molecules in the particular organ enables the contaminated by lentiviruses monocytes (outside the organs) to be inside the organs /8-11/ (“Troyan horse” model -/12/) and only the virus infected macrophages (monocytes) moving from the blood, penetrated into the brain /10,11/ and other tissues /11/. The macrophages, contaminated with SIV (simian Immunodeficiency virus) were found only around small venules /13/ (It means: on the way). Moreover,- the virus entry into central nervous system takes place early in infections /10,13/ that confirms a contamination of the moving at 1st stage macrophages In confirrnation, the. inoculated, in brain, HIV does not produce here the contaminated macrophages /9/.

The lesions in target organs for different lentiviruses (as HIV, SIV, Visna and CAEV) are similar /8/. Justly, one can explain that such contamination of the motile monocytes/macrophages is happen due to the rearward migration of the cross-linked endogeneous proteins on the dorsal surface with the consequent endocytosis /14,15/. Such directed migration is coordinated with the consecutive contractions (“centripetal movements”) of the microfilament sheath in direction of the nucleus (“waves of Heath”) /16/. In reality, the aggregates are guided (layer by layer) with the circumferential microfilamerits, contraction of which, justly, resemble the classical (Haxdey's) ones of the skeletal muscles (created later in evolution) (due to very special “imaginative” convergent molecular events /Zagyansky,Y. Patent Application FR-93-11198, pp.1-300, retired.; Annex AIII./.

§2.Cytokines as the Intermediates During Signalisations After HIV Infections.

Also, during the 1st phase there is an activation par env gpt20 proteins of the secretion of cytokines: TNF-α, IL-113 and GM-CSF /17-19/ that is independent on the virus entry /20/. However this production must be limited because a binding of HIV gpl2O proteins to the CD4 molecules could be insufficient to induce it /17/. A production of the ceramide at seropositives /21/ confirms an action of TNF-α. During the acute phase, the TNF-α secretion is proportional to an appearance of lentivirus antigens /22/. Justly a secretion of this cytokine inducts an appearance of the adhesion molecules in target organs /10/, reflecting a presence of only (almost) contaminated macrophages in the corresponding organs /10/.

In the case of a presence of the anti-env antibodies, a situation in vivo is quite different qualitatively. Logically, the Fc receptor helps to the virus to penetrate directly into cell by means of a fusion of the viral envelope membrane with the plasma membrane. There is a number of cases of the supposed enhancement, with the antibodies, of the viral infection in vitro and even in vivo /23-26/. The strong signal (as mitosis) must be essential for the real virus infection /27/. A number of factors increases justly a quantity of Fc receptors: interferon-γ (IFN-γ), macrophage (granulocyte-macrophage)-colony stimulation factors IM(GM)-CSF/, cytomegalovirus (CMV) /28,29/. So the IFN-γ without antibodies (in vitro) does not provoke an increase of the virus multiplication /30/. The CSFs make it through an influence on the IFN-γ respone region of the FcR (receptor Fc) gene promoter /31/ or through an Increase of a quantity of the cells with FcγR(III) receptor /32/. Naturally, the CMV has no effect at the 1st step but has it at the 2nd step (AIDS) where it decreases significantly a time of survival /33,34/, being the cause of the serious morbidity in the advanced HIV infection /35/. A quantity of cytokines during this 2nd step is well elevated /36,11/. The Fc receptor activation Induces the Fas ligand synthesis /37/. The Fas receptor-Fas ligand complex can produce a complementary signal, stimulating a producton of IL-2 interleukine (important at last stages of B-T cells interactions- FIG. 1), TNF-α, IFN-γ /38,39/ in cooperation with TCR (T-cell receptor) /38/ (it means: with CD4) /40/.

The FcR induces the cytokine synthesis (as TNF-α or IL-1), that activates the complete /4/ signal machinery /41-44/, according to the activated elements /17/. The TNF-α induces the HIV production through the activated NF-kB (by transcription), making also the new TNF-α /42,45/.

§3.CD4-Envelope Interactions. Absolutely New Molecular Bases of Their Interactions. General Universal Law of Switching of the Surface Receptors.

An interaction between the CD4 molecule and viral envelope proteins are rnultimolecular /46/ that permits to make the stonger conformational change of CD4 /47/. As result of this conformational change, the C04 glycoprotein must have a special conformation enable it to interact with glycosphingolipids and phospholipids in creating the receptor signalling complex (sometimes with dimerisation for small receptors as EGF one) /4/. To exclude the occational switching, such conformation must overcome the threshold of activation (maximum of free energy) because it is difficult to imagine such important special conformation as correct if there is the continuous spectrum of the energy states. And I show the Universal special structure, yet unknown, which make such discrete state.

Particularities of the general structure of the CD4 molecule resemble ones of the membranous IgG- the essential part of FcR /48/ and of the receptor for antigen- of B cells /49/. There is the hinge region between the domains D2 and D3 /immunoglobulin (Ig)- like/ /46,50,51/. The regions Di and D2 and also D3 and D4 are relatively rigid (like Fab and Fc fragments of IgG) 150,51/Like in IgG, there is always the bond S—S near this hinge region /52/. The hinge region play the very important role in the total structural integrity of CD4 /51/. And one supposed that “flexion about this “hinge” might be an essential post attachment event” /46/. In the case of lgG Ihere is the discrete dissociation of the Little special Protein (˜1.5 kDa) (CH2 regions). (I propose to name this extremely important protein: Du-2T according to the names of its Discoverers /53,54/). This dissociation provokes an appearance of a more rigid state 153,54/ with conformatibnal changes /Refs.3/.1 very clearly confirmed (directly and strongly) by Dandliker laboratory /55/. And again, more directly, it was proved (with direct continuous registration of fluorescence polarisation during cdnformational changes) by S. Shanin and Y. Zagyansky (unpublished, 1974). When we only were adding small portions of hapten DNP (dinitrophenol) to pure. anti-DNP-antibodies (labeled already nonspecifically covalently with dye dansyl), the polarisation was always growing up step by step (until saturation). It is very interesting: such effect was undoubtly obtained only with complete pool of the fractions of antibodies during last step of purification. In the case of separate fractions there was no any change of polarisation (justly because of evident lost of small Du-2T, a dissociation of which changes already such conformational state for an essentially more rigid /53,54-1981/, where the Fab fragments interact with the Fc In bending the hinge region in two parts-“scorpion” structure /3/). Because the IgG is also the switching part of the membranous Fc receptors and receptors for antigen of the B cells, one can predict a presence of such little fundamental protein for Fc receptors (and receptors for antigen of B-cells) /3/ and also for receptors CD4- like (in above sense) and other receptors of Ig family like ICAM-1 (intercellular adhesion molecule) also having the hinge region between two globular parts, each one relatively rigid with close S—S bond /56/. The fundamental proofs. of Universality (by Universality!) of such general very important mechanism is given below (Parts VII-X).

Consequently, an interaction with protein env of HIV switches the discrete state of CD4 (as well of env viral proteins)(proved definitively in Parts VII-X), that permits, justly, to interact with glycolipids with help of the homologous intercarbohydrate interactions /4/ in making the complexes: glycoreceptor→gangliosides→cerebrosides→→sphingomyelin→cholesterol→ . . . with phase separations 141. Such complexes make some aggregations with unification of the same phases.

But because of low concentratiohs of the viral particles during the natural contamination of man this microaggregation is not sufficient to provoke a creation of the farges patches, conducted already by the microfilaments for the capping and endocytosis /57/ and this is a clear reason that a grand majority of the monocytes and lymphocytes are not contaminated at the 1st stage. But during the cell movement there is a creation of the comrmon patches (transfonned in COMMON endocyted cap) Including the CD4-env and other “cross-linked” proteins from the renewing parts of the moving cell (as larnellipodium, focal contacts, focal adhesions, close adhesions and tail) that makes microaggregates with local phase separations /4/ unified with help of interactions between the glycolipids and proteoglycans (including syndecans) where justly the common carbohydrates/galactosyl(β1-3)N-acetyigalactosamine/ between the gangliosides (on the outside part of each complex) and the unifying proteoglycans (bridges) /58/ permit them to aggregate until the critical size where the special proteoglycan syndecan at increased local threshold concentrations could already interact with microfilaments /59/. in reality, the state permitting to glycoreceptors to make the intercarbohrdrate homologous interactionIs is created even after an interaction with the good monovalent ligand as the hormone and, normally, the divalent ligands make oinly the stronger conformational change in. cross-linking the parts of the same molecule /4/. From this size of patches there is already the directed nperistaltic^{T} propulsion of such complexes with a help of the hiicrofilaments and energy until the perinuclear zone with final endocytosis /14,15,571. Naturally, the complete mechanisme of cell movement was resolved as application. of the profound, principally new, mechanism of cell signalling and new molecular LAWS /IntemationaL Patent Applications No.PCT/FR94/01159; PCT/FR95/1300- both retired/).

So only by this way with low concentrations In the organism, the HIV can penetrate, into the cell by the endocytosis (without antibodies). Indeed, the increased movement of the monocytes is correlated with the increased ingestion (“phagocytosis” according their term) of the small yeast cells /60/ or of relatively srnall bacterium. extracts /61/ and also the more active (and more mobile, correspondingly) neonatal monocytes are, easier infected in vitro /62/.

§4.Cytokine Receptors are Responsable for 1st Entry of HIV Viruses into the Monocytes/Macrophages During Their Movement.

There is a number of cytokines (β) that attracts the monocytes having the corresponding high affinity receptors /B3-68/, participating in the macrophage recruitment within tissues /66/. These chemokines are released by the infected (HIV) macrophages /66,67/ or by immunocompetent cells as Nuclear Killer (NK) or T8 cells after a release of the special cytokines (as TNF-α or IL-1β) by the monocytes after an injection /68,69/. These β-cytokine receptors also can activate cells /66/, whereas in the T4 cell case -(in vitro) and the monocytes/macrophages, they coinfect these cells with HIV /84,65/. But in the case of the inactive β-chemokine receptor (CCR5-2 allele) there is no appearance of the anti-HIV seropositivity in vivo (and, evidently /I/, AIDS) /65/ that means (together with an absence of the HIV virus infection in such cells /65/) the virus entry absence in macrophages in vivo at the 1st stage where the CCR5 receptor was responsable for the HIV entry /see also 64/. The fact that the chemokine binding to this CCR5 receptor inhibits (blocks) the CD4 cell infection in vitro /64,65/, confirms well the macrophage role as the 1st principal target of the HIV virus. Similar cytokine receptors for the 1st entry of other viruses during the macrophage movements also exist /70/.

Appendix. Naturally, the Universality of such new state transitions clearly follows from Universal mechanism of the protein foldings with chaperon help: in endoplasmic reticulum-Golgi (and in cytoplasm also) (Parts VII-X). The tensional folding of membranous proteins usually takes place at latest, stages with peptidyl prolyl isomerase and protein disulfide isomerase and peptide (obtained after limited proteolysis of N- or C- extremity) help with presence of two prolines (for folding with two trans-cis isomerisations), the loop created with disulfide bond and two carbohydrate homologous neighbour oligomers (to -protect the dissociation of Du-2T- like protein). The 2nd state (so called “scrapie”) of prione protein (also more rigid!) is created justly because of the dissociation of Du-2T like proteins during signalling (Part X) with consecutive homologous intercarbohydrate interactions with bond crosslinkings during the signalling in the zone of principal receptors /4/. The same signalling is after translocation of the specific peptide for T-cell receptor (TCR) from its not very specific complex with MHC class I (Part IX), where exceptionally, there is a presence of destabilizing charges in the hydrophobic Intramembranous part of TCR /71/. The presence of 2prolines and 2 potential 0sites (that logically, contains Du-2T-like protein), confirms above conclusions. Consequently, the anti-D3 domain antibodies stop the CD4 molecule signalling (with gpl2O) /74/ and the D2 domain is also important for HIV infectivity /75/ because of direct interaction between D2 and D3 domains (as direct Fab-Fc interactions with Du-2T dissociation /3/) during the bending confirming clearly the IgG “scorpion” structure during antigen binding /3/. The similar nearby presence of 2 prolines and 2 N-chain potential sites at D3 regions of ICAM-1 /76/ and the presence of the bent of 90° between D2 and D3 domains /77/ confirm above data. The absolutely certain proof of such SPECIAL protein functionings was possible only due to profoud UNIVERSAL laws of protein foldings resolved by me (Parts VII-X).

PART II: “AIDS to the End. Molecular Bases of HIV Interaction with Target Cells and General Scheme of AIDS Development”.

§1. Proofs of a Delayed Anti-Env Antibody Production with Help of the New Law of NK-Cell Selections.

There is another /1/ quite different molecular proof that the antibodies against the env proteins (anti-env) appear later. For this important purpose, I shall resolve the absolutely new great mechanism of the selection of the nuclear killer (NK) cells, based on the new law of the intercarbohydrate homologous interactions. Moreover, this new law permits to explain the important mechanisms of the Infant AIDS, the “strange” AIDS absence at chimpanzees, the net protein action, the 2nd (and 1st) HIV penetration into the cell and structure of the vaccine “actions”.

Analogically to the fundamental switching due to an interation between o-chains of the HIV env and CD4 molecules /1/, the Interactions between O-chains of the NK special receptor (NKR-Pl) and ones of molecules of target cells must take place. The NKR-P1 receptor belongs to the family of the Ca dependent animal lectins (C-type) with carbohydrate recognition domains (CRD) /2/.

A presence of the 0- linked chains at the mucin- like counter-receptors of the selectins (C- type lectins) is established 13/. Moreover, there is a direct presence of 0 linked glycans In the string region at CRD of the mannose receptors /4/ (justly group E of C- type lectin /5/). One can see numerous O- linked potential sites /6/ in a number of CRD (C-type) situated also at N- end 717 as well in the NKR-Pi receptor (all gene variants) at a beginning of CRD domain /8/. Moreover, it is shown (if correct) that the NKR-P1 (even without Its classical N- chains) interacts with different carbohydrates, especially with ones containing galactose-N-acetytemine (GaINAc) (gangliosides) and glycosaminoglycans (GAG) /9/. The GaINAc is justly essential component of O- chains, and a presence of the GAG chains is also very natural in NKR-Pi because of their established presence at C- type fectins /2/. Moreover, there is a presence of classical potential sites (SerGly) with charged negative amino acids nearby 110/ for GAG chain at C- domain of NKR-PL /8/. The authors /9/ utilized the recombinant NKR-P1 (bacterium) where a synthesis of N and O- oligosaccharide chains (including GaINAc) takes place near the cell surface /11/ and a synthesis of GAG chains also takes place /12/.

The clear rules satisfy a grand majority of corresponding sites established even at low degrees of O- glycosylations /8,12,13/. Such data were established by the well developed new valid method that can establish even a low percent of O-glycosylations /12/ in difference with a number of more ancien data made with similar to each other methods and potential errors /12/ where the errors in situ were CATASTROPHIC. For instance, it was found 1 O-site instead of 7 real sites /14/ or zero O-sites instead of 10 and 6 sites /15/. Also these data were specially established only on the open parts of proteins in difference with many other data with unaccessible to solvent parts which could not be accessible for the O-glycosylation enzymes /16/.

This perfectly coincides with biological sense of such rules. There is a presence of several (at least 3) GaINAc-transferases with different specificities /17/. Also sequence differences in the cytoplasrnic tail and stem region of glycosyitransferases indicate their different colocalization where the stem region permits a better access to protein substrates /18/. The influence of the nearby lHe for glycosylation is excellently confirmed with quite different results: the excellent substrate for T3 was the V3 loop of HIV gp120 (membranous protein) that clearly was not glycosylated by T1 and T2 ones /17/. And in V3 there is justly a permanent presence of the nearby lie /17,19/ lunglycosylable in situ by transferases (T1 and T2)/.

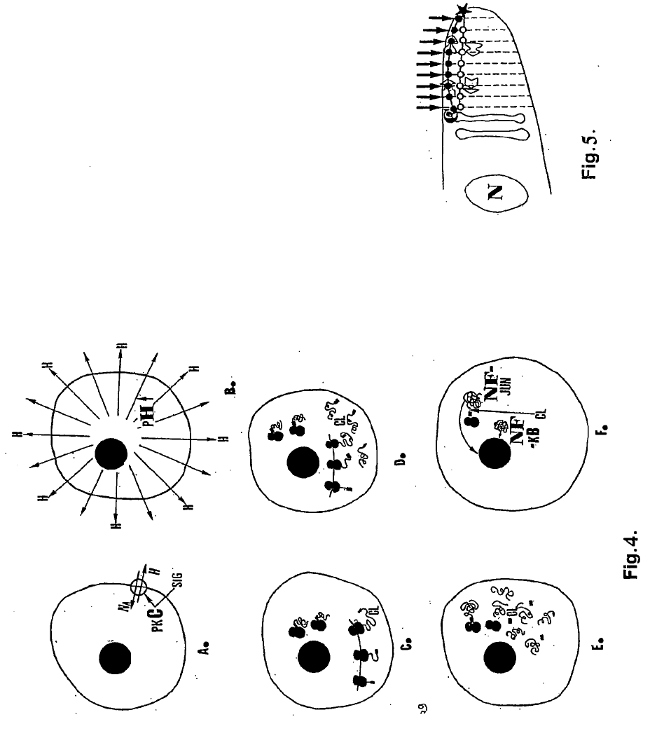

According to /20/ there is an absence of short O-glycans at the V3 loop but the “more extended structure than GalNAc or NeuNAc-GaINAc” could be present. A presence of the long O-glycans in high concentrations, clearly visible in glycoproteln gpl20, can be easily concluded due to a clear shoulder at higher molecular weight peak /21- FIG. 4/. So a presence of the longer O-glycan in the V3 gp120 loop, having the nearby lie at the glycosylated threonine, is supported.

One can easily see a synthesis of core 2 O-glycans after transection of the core 2 β1→6N-acetylglucosaminotransferase (C2GnT) in T-cells where the mass of CD43 (leukosialin) essentially increases (15 kDa) and one of CD45, CD44 and receptor-linked protein-tyrosine phosphatase also increased (3-5 kDa) /22/. The CD43 has a number of potential sites with Thr with the nearby lie /23/ and all these other proteins have several such sites /Refs.24;25,26/. The long O-chains are synthesized on the above CD45 /271 and justly a branch of core 2 O-glycans could be long due to poly-N-acetyllactosamninyt /28/.

Moreover, there is the endogenous “lectin” galectin-1 that bind specifically to these core O-glycans and to the above CD43 and CD45 /29/. But justly this galectin has several O-glycosylation potential sites with nearby lie /30/ that conftirms clearly the homologous intercarbohydrate interactions and the developed rules of O-glycosylation that are very important for the proofs of the NK-cell selections. Moreover, the sensible glycosylation takes place at the extended, accessible, relatively short, sequence of membrane glycoproteins separating globular domains or globular domain with membrane bound domain /6,31-33/. So here there is no problem with an accessibility for potential Gsite /16/.

Consequently, taking clear confirmed Rules for potential glycosylation /6/ for correctly mesured data also with medium and low glycosylations /12,34/, one can well determine sites of potential O-glycosylations, possibly with some necessary small corrections 131,35/, and determine the restricted for O-glycosylations residues Inside the protein /161. Anyway, I investigate, in this work, only the open loop parts of proteins, so with used Rules, almost all predicted sites must be correct. The good convergent confirmation of these rules from quite different data, one can see from the clone changes after 1st HIV (SIV) contamination where the insistent alterations justly of potential O-glycosylation sites (as well N-glycosylation sites) 136/ are in accord with work /6/.

So the homologous interactions between O- and between GAG chains of NKR-P1 receptor and surface molecules of target cells must take place reflecting such strong mesured interaction /9/. A strong conservation of S—S bonds at CRD domain /2/ and multispecificity of oligosaccharide binding support the correct specificity, confirming the above data with presence of Du-2T- like protein.

Interactions between NK and target cell glycoproteins are species specific /37/ as well cell specific /38/. This welt coincides with “strange” dissapearing of NK1.1. specific determinant of mice NKR-PI receptor after mouse receptor gene expression in other species as well justly in genetically different mice strains /39/.

So a mechanism of a new kind of selection of NK cells is established here. Firstly, being present in minor quantities, each NK- cell type from a large complex pattern, at once, searches the target cells with the similar “foreign” carbohydrate pattern (including justly very different cancerous carbohydrate pattern /38/) on the cell surface. Evidently, an elimination of NK- cells with “self” organism pattern of carbohydrates due to the apoptosis must take place during the embryogenesis After such melting, the target cell is lysed /9,37/ due to, firstly, the melting of the plta membrane with help of the carbohydrate homologous interactions /1/ and then the activated NK- cells undergo a proliferation /37/.

An absolutely necessary heterogeneity of NKR-P1 receptors (that are not expressed in other tissues!) with special mechanisms (inculding established small deletions of DNA coding exterior regions), that must exist for such diversity, was already proposed /39/. Evidently, such small differences in primary structures must be translated also into differences in the carbohydrate chain pattern.

So according this new important law, a presence of the “foreign” pattern of carbohydrates on the cell surface must determine the multiplication of NK- celIs. And justly a strong presence of NK- cell lysis activity against the env proteins and its absence in the case of the gag proteins or reverse transcriptase confinns a presence of the env glycproteins on the cell surface /40-43/ /this activity can be also due to specific antibodies at special Fc receptor- (CD16) but it must happen later /44/, after already appearance of these antibodies/. If to make the cell synthesis of the mutant env proteins without carbohydrates, the env is absent on the cell surface and there is no NK specific anti-env answer but the CTL answer /45/. So a continuous presence of the HIV env proteins on the cell surface is once more evident and, consequently, the. triggering of the anti-env antibodies must be. delayed in comparison with other viral proteins that are quickly exocytosed outside (in part) (including nonstructural proteins). So the another molecular evidence of late anti-env appearance is presented.

§2. Intercarbohydrate Mechanism of gp120-CD4 Interactions.

It is very Important to know that such viral penetration-.must be due to the intercarbohydrate homologous interactions /i/ where the consequent local dehydration destabilizes the polar plasma membrane, provoking the fusion /i/. A presence Qf other (than CM4) molecules is supported with fact of an absence of mouse cells contamination by HIV (including CD4 cells transfected with human CD4 /46/). An interaction of the very havily glycosylated HIV env proteins with essentially weaker glycosylated CD4 molecules and other glycoreceptors as Fc and cellular glycosphingolipids /47/ and proteoglycans in the case of the direct fusion /1/ or of the chemoreceptors (with CD4 molecule) with a network of proteoglycans and glycolipids in the case of the motility with endocytosis /1/ leads to the fusion. This fusion “can be influenced by the pattern of viral envelope glycosylation” /48/ and infection conditions suppose very high concentrations of gp120. A role of carbohydrates in an interaction between gp120 and CD4 was already proposed /49/. A structure of the N-linked carbohydrate chains of CE4 and gp120 is similar /50,51/. Even a loss of a single glycosylatlon site of HIV-2 diminishes its binding to CD4 at let 50-fold /52/.

Generally, a pattern of carbohydrates and infectivity depend on a type of host cell /38/ and particularly, there is a presence of the chains with lactosaminoglycans on HIV gp120 only at macrophages, the isolates from which have justly a better infectivity /53/, confirming such thesis and a predominate role of macrophages in infection /1/.

Fortunately, the developed data permit to prove the general model of the HIV env glycoproteins and CD4 interactions with subsequent activation accompanying by a serie of conformational changes allowing the fusion 54/. Logically, the intercarbohydrate homologous Interactions between O-chains of the V3 HIV region and O-chains of CD4 must be present provoking the main signal although the massive interactions between their N-oligosaccharides must also take place especially during fusion. Even an elimination of only N- linked carbohydrates (although the 0-linked ones rest in situ) /55/, eliminates a binding between CD4 molecules and nonglycosylated (almost) envelope gp120/45/. A general presence of O-chains in CD4 molecules is proven with help of the very specific lectin jacalin (specificity: 3-Gal(1-3)-βGalNAc) /56/.

Moreover, one can see the very strong potential persistent O-glycosylation sites /57/ near justly the hinge and S—S bond at human, rat and mouse CD4 molecules /58,59/ always with the nearby Ile characterizing a presence of (as well) core-2 O-glycans (see below). An absence of an inhibition of the CD4 interaction with gp120 by jacalin /58,60/ confirms a presence of other, core 2, O-glycans. Moreover, an importance of the hinge region in the CM4 interaction with MHC-II molecules is established /61/ and the similar structural unit of CD8 molecule must interact with MHC-I molecules also /61/. But justly the hinge region of CD8 is well O-glycosylated with a site with the nearby Ile /33,341 confirming the O-glycosylation (including core 2-O-glycansl) of CD4 hinge.

There is also the presence of 8 O-linked chains in gp160/55/ and 4-8 such chains in gp120 /62/. A persistent presence of O-linked glycosylation of other retroviral env proteins /55,63,64/ indicates more general conclusions. A presence of the O-linked chain at V3 region of gp120 is once more too probable /65/ (§1). The V3 loop was consistently glycosylated with GalNAC, Ciinked to threonine 324 which is conserved feature of all gp120 V3 loops sequences /65/. This Thr 324 is situated justly at the region of CD4 binding and justly satisfates exceptionally very well to a number of conditions of O-glycosylation for threonine /57/ and evidently an utilisation of different antibodies against different epitopes (including the O-linked oligosaccharide) can give variable results due to their different effects on a amplification of an infectivity due to the Fc receptor /48,88/.

In a region of the very specific O-saccharide binding /βGal(1-3)-βGalNAc/67/ of the lectin jacalin there is the Thr 92 presence with two lysines at N-side (but without nearby Ile) /60/ well satisfating to the O-glycosylation conditions /57/ without core 2-O-glycosylatlon. As confirmation, the lacalin interacts with IgA1 /68/ having the GalNAc—Gal chains /69/. Moreover, the major α-subunits of lacalin have the carbohydrates that escape a microsequencing for N-glyccsylation /70/.

Naturally, such high selective specificity of this lectin could be explained with the homologous intercarbohydrate interactions /47/ due to a presence of its own O-chain βGal(1-3)-βGalNAc and the same could be at Thr 299 of C2 gp120 region and the core 2 O-chain could be at Thr 283 (with nearby Ile!) /71/. A help of different groups for binding /72/ is not excluded although a good affinity for disaccharide βGal(1-3)-βGalNAc (that must be very selective) is not so obvious from above data /72/. So from these data, one can confirm the important, for infectivity, interaction between C2 and V3 regions of gp120 with a prostration of V3 that has been observed and supposed to be important for fusion /65,73/ and these Interactions must take place justly due to intercarbohydrate interactions of these O-chains so Important for infectivity /65/.

Logically, during interaction CD4- gp120 there is, in particular, a creation of new interactions between one (of interacting) O-chains of CD4 (near hinge) and the V3 gp120 (Thr 324) /74/ with conformational change of CD4 and gp120 /54/ with disruption of carbohydrate bonds between C2 and V3 of gp120 and similar INTRAmolecular CD4 bonds and conformational change of CD4 and env. A dissociation of Du-2T- like proteins from CD4 and env, fixing the discrete conformational change, switches a possibility of change of the INTRAmolecular (intra-CD4, intra-env) intercarbohydrate homologous interactions for INTERmolecular ones (inter- CD4-env) (Parts VII-X). Such serie of conformational changes must allow a consequent fusion /54/. The fact, that the jacalin. is mitotic only for CD4 cells although it binds to all blood sells /75/, confirms such switching mechanism with sensible region. The above results accords with the “hysteresis” connected with glycosylation of env proteins /76/ (with formation of stable S—S bonds /77/) and interaction CD4-gp120 with “DU-2T”.

§3. Basis of HIV-CI D4 Interactions: New Law of Intercarbohydrate Homologous Interactions.

Because the very important New Universal law of intercarbohydrate homologous interactions is very important for understanding of the molecular basis of the gp120 CD4 interactions and the virus penetration into the cell (established earlier) and also for complex mechanism of Infant AIDS, “strange” absence of AIDS stage at chimpanzee and other problems, I briefly present this law, never published in scientific literature.

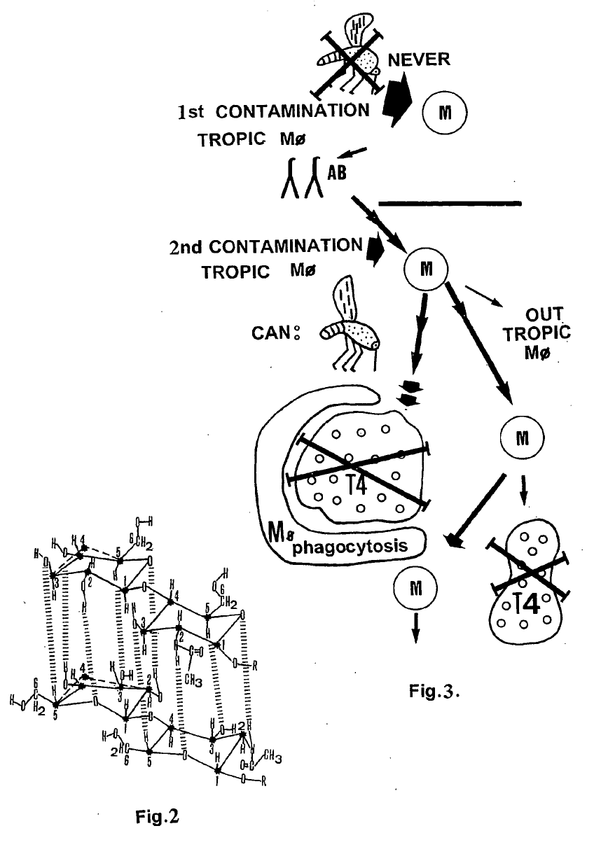

Because of a presence of the hydrophobic glycopyranose ring, the carbohydrate molecule must have amphipatic properties. It means that the conditions could be created when the close superpositions of the identic hydrophobic (which means that they contact each other because they do not interact well with water) glycopyranose rings will permit to eliminate the surrounding solvent molecules. The intramolecular H-bonds are destroyed already in water solution /78/. Evidently, the optimal best fitting structure must be created with help of mainly carbohydrate hydroxyl groups participating in strong hydrogen bonds /47/. Only chair (C1) conformation are normally preferred from two “chair” and six “boat” possible conformations /79/. But the hydroxyl groups (during the optimal interactions) have a tendency to take the equatorial position in making stronger H-bond interactions /80-82/ and such tendency overcomes a preference for the classical Cl chair conformation /79,Refs.78/ (FIG. 2). For β-D-glucose and D-xylose, the C1 conformation remains /79/. But the β-D-galactose pyranose “pays” for such tendency of its axial OH group on C(4) atom to be more equatorial by flattening of its C1 conformation that produces a distortion of its C1 conformation /78/.

So one can see that the typical terminal cell membrane sugars /83/ have the normal C1 conformation (creating of selection?) with all equatorial OH substituent groups /83/ (as N-acetylglucosamine or α-sialic acid) or some flattened C1-derived conformation due to a tendency of one resting vial hydroxyl group (according to their normal C1 chair structure) to become equatorial (as β-galactose or N-acetylgalactosamine) (FIG. 2). Evidently, the divalent ions as Ca potentiate these interactions because of their capacity to dehydrate the area near the interacting surfaces /84/.

A number of cases with the fit interactions of homologous oligosaccharide chains are found already. S.Roth and S.Roseman clearly showed the synthesis of the cell surface carbohydrate molecules, ,ustly homologous to ones of the cell substrate /85/. There is already a number data concerning the intercarbohydrate aggregations of glycosaminoglycans /86/. In the immunoglobulins, G there is the “clear evidence for direct interaction between N-linked oligosaccharides” that are identical /38/ and this interaction, justly, is extremely important in the complement activation /87/. But the lectin properties of deglycosylated IgG are well unknown /38/. There are also homologous interactions between smaller O-chains of IgA and IgD that are also Important functionally /69/. Also the distal structures of gangliosides resemble the distal carbohydrates of glycochains of proteins /88/ and the cerebrosides resemble the proxdmal carbohydrates of gangliosides /47/ that permits to have the chain of glycoprotein receptor complexes: glycoproteiroangliosides→cerebrosides→→(sphingomyelin, cholesterol . . . ) /47/. Yet in 1989, Hakomori and coworkers found that there is the good Interaction between, justly, the same carbohydrate chains Le^{X} /89/. And here it is logically shown that the “lectin” galectin-1 has the same type Core 2 O-chain as the galectin interacting proteins (CD43 and CD45) (according also primary structures) (§1). And the CD4 molecule interacts with V3 and C2 HIV gpl2o regions with help of homologous intercarbohydrate Interactions (§2). A correct finding of numerous oligochains and their analogous counter-chains represent a work relatively simple today /12/.

Appendix

At ending of writing these inventions, the strange work with the (same) author (who suddenly denied without real reasons his ovjn good results) appeared /90/. The rules of the same Dr.Gooley with coworkers, according primary structures with established already sites in proteins gave good results (133 predicted threonines from 161 really determined—83%!) /91/. It could be even better than for N-glycosylation rules with some complications /12, 13/ where also one cannot deny N-glycosylation rules because the N-glycosylation sites cannot be gtycosylated for cytoplasmiic proteins. For instance, the cytoplasmic translation initiation factor has 4 N-glycosylation sites /92/ which have to be free. Moreover, for serlne sites, this author (very recently) defended his rules: “X—P—X—X motif provokes glycosylation of serine and threonine” /31/.

Elementarty, these authors strangely consider the repertoires of “GaINAc-transferase(s) in human milk” /90/ (excreted occasionally), used in vitro, equal to those of constitutive enzymes In Golgi of mammary cells in vivo. Moreover, “altered enzyme specificity as result of inappropriate solution conditions, absence of cofactors (and of special Golgi structures) . . . , the result of artefacts resulting from the use of relatively small peptides containing charged N and C ternini” /93/, presence of at least 4 GaINAc-transferases /94/ with attainable mutual transferases agglomeration in line of chain glycosylation and a stricter secondary structure of peptides in proteins /93/ with sites for enzyme attachment underline the authors! conclusions /901 as purely incorrect.

Moreover, only for determined in vivo O-sites for globular integral membranous glycoproteins (75 sites) /91,31/, one can see that more than 90% sites are well predicted according rules Gooley and coworkers /6, 12, 13, 31, 33, 34, 57/ worked with “the most effective approach” /93/. Moreover, there is no mystery about “exceptional” glycosylations by GalNAc-glycosyltransferases. A presence of nearby perturbing proline arguments for such special conditions for this glycosylation. The positive charge is also important for glycosylation /33,91/ and nearby negative aminoacids make the negative effect /13,35/. Clearly, a very net necessary presence of justly oppositely charged negative residues and very weak necessary proline presence for CONCURRENT glycosaminoglycan glycosylation /95/ brilliantly confirms these data. Logically, the OH-groups of Thr and Ser sites must be the epitopes /93/ for common Thr/Ser specific GaINAc-transferases. Moreover, the site with Ile must be, logically, glycosylated with important Core 2 chains that are made with help of T3- GaINAc transferase (§1), logically, having a specific complex with other transferases making such chains (especially on membranous proteins) and such Ile, logically, Inhibits other GaINAc transferases (T1 and T2) (g1). Another fact confirms a role of Ile for Core 2 selective glycosylation: sequence (corrected) of N-acetylglucosamine- specific receptor (NAGR1) has a numberof potential sites for O-glycosylation justly with Ile (for Core 2 chains with GIcNAc) justly in Ca-coordinated domain (where Ca is necessary for intercarbohydrate homologous interactions) /96/. However one cannot exclude here the core 3 and 4 chains /97/ although, visibly, “much less abundant” /98,991. Certainly, the data,with clearly well glycosylable in vitro site in V3 gp120 HIV region by purified mammary T3-transferase (on large peptide), relatively the same site, completely unglycosylable by T1 and T2 transferases, are correct, moreover with other data (§1). A prediction of O-sites using networks /91/ is made with a number of errors /§1 ,93/. Consequently, there is “the inhability for (such) prediction to resonably predict the PSM glycosylation pattern” /93/ and it reflects the basic fact that almost each (???) site has some probability to be glycosylated /54/ that is principally wrong.

There are general numerous errors with recombinant proteins Such pure error with recombinant in bacterium proteins due to ignorated O-, N- and GAG- glycosylations was already discussed (§1 ). The proved here data permit to explain another kind of spread errors. The synthesis of proteins with help of vector constructs in other species cell could give quite different glycosylation pattern that depend on species, organ and cell /17,38,100/ that is so important for function /§1, 1/. Evidently, the synthesis in insect Sf9 cell give quite different pattern, so, for instance, the purified porcine. GaINAc-T1 “exhibits very high substrate specificity for human erythropoietin sequence” but “recombinant (in insect cells) GaINAc-T1 is devoid of such activity” /100/. Because of close evolutionary relations, the cloned in monkey Cos-7 cell, GaINAc-T1 gives similar results with the purified bovine GaINAc-T1 /101/. So such very spread source of errors must be evoided.

Part III: “AIDS: to the End. Two Special Pathways of Intracellular HIV Development in Vivo and Their Important Consequences.

§1. Molecular Proofs of Two Types of HIV Entry. Intracellular A-particles are a Clear Reason of False Viral Presences after 1st Contaimination (Seropositives).

It is known that a number of viruses enter the cell by direct fusion and by endocytosis via coated pits /1/. In the case of the HIV virus, one can see the same double picture during artificial contamination of cells in vitro /2/ but only the direct fusion of HIV with cultured cells is sufficient for infection /3/. During the 1st contamination in vivo (seropositives), the primary Langerhans cells (macrophages) were “infected” by viral particles with internalization /4/.

And the virus entry before asymptotic phase, without Fc receptor help and strong signal and with endocytosis, provokes weaker “replication” rates with lower levels of the viral RNA and proteins than during the AIDS phase /5, 6/ and the replicated DNA is not integrated /7/. A majority of the EIAV virus DNA is unintegrated on 16th day after inoculation /8/. A restriction of replication of particles from asymptomatics is determined by events before provirus formation /9/. whereas the cells from asmptomatics have, generally, the unintegrated DNA /10/. The viral unintegrated DNA forms exist as the multiprotein complex that is different from the preintegrated complex of HIV-1 /11/ and the viral genoms of particles, -entered Into questlent cells (by endocytosis), are not completely transcribed /12, 13/. Such unintegrated forms of HIV DNA in infected cells do not lead to a production of infective virions and serve as a template for viral RNA and protein synthesis /14/.

The vif viral protein, for Instance, is lrilportant for the early event after virus entry resulting in reverse transcriptase activity /15/. The unintegrated lentiviruses DNA produces a large number of defective RNA which could be packed in “virions” without env proteins /13, 16-20/. During 5 years of consecutive observations after the seroconversion (primary infection), the heterogeneity (in vivo) did not changed /17/. This heterogeneity was found by observation of the direct V3 (gpl2o) region variation but not by polymerase chain reaction that has difficultes to distinguish between integrated and unintegrated DNA /23/ (although in confirmation, see also /24/).

But the DNA integration is necessary for productive infection /25-27/ and a quanity of the integrated HIV DNA increases at AIDS patient /10/ (comparing with level of previous 1st contamination). A presence of increased quantities of integrated DNA at encephalitis /28/ indicates that the strong 1st contamination leads to the strong antibodies answer with numerous arrived already macrophages and lymphocytes that permits the intensive lesion creations in brain at the 2nd contamination conducting to the encephalitis /29/.

Justly in the case of 1st nonproductive contamination, the defective gag proteins make the capsules (with RNA) that are localazed in the vacuole- like structures near endoplasmic reticulum /30, 31/ although normally, this C-type virus /32/ aggregates near the integrated in plasma membrane env proteins Similar particles buddings in the endoplasmic reticulum lumen are obtained with vectors carrying the gag polyproteins of the tentiviruses with mutations at their N-end /22, 32-35/. These capsules well resemble the noninfectious spherical intracystemal A particles (IAP) /21, 35 36/. Even the primary structure of the special transcriptional HIV regulator, remplacing the rev protein (it means: at acute stage), is similar to that found in murine IAP /36/. Similar Pr55(gag) particles, sensitive to the protease, could be synthesized even with rabbit reticulocyte lysate /37/.

Already at the AIDS stage (productive Infection.) there is the Budding of viruses in patient cells of the macrophage lineage /38/. A change from so called non-syncytium Isolates to syncytium-Isolates types production correlates with disease progression and the CD4 cell count changes are a bad marker in all asymptotic phase (being always in “nornmal range”) /39/ until the 2nd contamination and productive infection. As in the case of vectors in the eucaryote cells with normal polygag protein, an assemblage of normal capsules takes place near the plasma membrane at the AIDS stage /34, 35/.

The gag myristate for the retrovirus particle (C-type) assemblage facilitates the gag protein membrane association /40, 41/ although several retroviruses as EIAV do not have the gag myristate at all /32/. The direct proof, that the gag interactions are first ones, is absent /32/ because it is difficult to imagine their synthesis at endoplasmic reticulum but their first interactions somewhere else, near plasma membrane (for C-type viruses) /32/. In reality, it must be the ribosomes that are transported to the plasma membrane that is known for instance, for β-actin (plasma membrane or growth axon cone) /42-44/. It is the 3′-region of mRNA that directs a localisation of ribosomes /45, 46/. It is shown that the retroviral particles (C-type) are mainly situated at plasma membrane with mnicrofilaments regions (cells without excessive gag concentrations) as the punctate pattern /47-50/ and the ribosomes make a complex with them /49, 50/.

It is known that a presence of the polyA mRNA tail correlates with mRNA displacement to the growth cone /43/ (and logically to the area. near plasma membrane). Naturally, this must be the case also with polygag. Its final mRNA is Justty well polyadenylated /51/, the higher structures of its mRNA are very sensitive to its primary structure, especially near 5′-end /52/ and a binding of the Poly(A) binding protein 1 to the poly(A) gag HIV mRNA tail increases a stability and translation of this mRNA /53/. Different localizations of ribosomes (at cytoskeleton: near endoplasmic reticulum or plasma membrane, at cytoplasme and on endoplasmic reticulum) clearly .must reflect different means of their transport from the nucleus and their living cycles (Annex. IA§6, IIA§3).

So all above numerous data clearly confirm one main thesis: a presence of 2 chains of events during an interaction of the HIV virus with corresponding cells During the nonproductive 1st interaction with help of CD4 molecules, the viral particles penetrate into cell with help of endocytosis during motility (logically with previous patching on the cell surface) with subsequent creation of the unintegrated DNA in nucleus, strong stable heterogeneity and exocytosis of different viral proteins (including regulatory but principally without env) and noninfectious pseudoparticles (easily proteolysable) with the gag coat in cytoplasm (IAP-like particles) containing however the viral RNA and reverse transcriptase. The very clear general delay of a creation of justly anti-env-antibodies, proved by me from quite different immunological data /54/, is too evident. The env proteins, becoming the integral proteins, must mainly wait a moment of cell phagocytosis by the very rear yet NK or Ts anti-env cells in difference with rapid presentation of other endogenous secretable proteins to the MHC-II molecules inside the cell (§4). During a productive infection with obligatory help of Fc receptors due to appeared (AFTER elimination of infectious viral particles of 1st contamination) anti-env antibodies, the viral particles penetrate into cells due to the direct fusion (strong concentrated membrane melting) with subsequent integration of the viral DNA, absence of heterogeneity and normal creation of the infectous particles near plasma membrane (C-type) (2nd contamination).

§2. Confirmations of Mechanisms of HIV Behavior with Zidovudine (AZT).

The well known “strange” experiments with zidovudine (AZT) strongly confirm above conclusions. Evidently, a stopping of the first viral transcription RNA-DNA or even the later process of divisions of the lymphocytes B and T during the antibody creation with help of the AZT leads to the “slower” clinical disease progression during new seroconversions (it means a reaction against the virus at a beginning of first virus entries) or to the well reduced perinatal transmission /55/. But, evidently,.a treatment of a patient clearly after seroconversion at steady state (where there is no reverse transcriptase actions) does not make any progress (in mesuring the p24 concentrations in serum) /56/. It is confirmed with HPA-23 (other inhibitor of reverse transcriptase) which does not influence seriously the patient p24 level before the AIDS stage /57/. The (uninfectlous) viral particles (pz4) are always present at asymptotic phase /58, 59/. But better survival after AZT application during the asymptotic phase was observed /60, 61, 62 (Refs inserted)/ justly due to a probability to avoid consequences of the 2nd contamination, proving its real presence. The negative results of other analogous (but very long) experiment /63/ are obtained because of only a probability of AZT stopping of the new contamination, where this probability (to stop all new contaminations where even only one is fatal), evidently diminishes with time due to a number of consecutive patient contaminations in his life.

But evidently, at the real AIDS “stage” (2nd consecutive contamination), with multiplications of viruses and contaminations of new cells, the AZT must diminish such multiplications at the moment /61, 82 (Refs inserted),63, 64/. Consequently, there are. the cells with integrated already viral DNA that “wait” to renew their mortal action after unavoidable decrease of AZT dozes which is very toxic /56, 61, 82 (Refs inserted),63, 64/.

The relative experiments of Gendelman and coworkers /85/ confirm also such conclusions where the AZT exclusively inhibits a multiplication of viruses in patient rnacrophages treated in vitro with MCSF (macrophage colony-stimulating factor) taken from late AIDS stages (5 and 6 in Walter Reed classification which is evidently not precise and has errors see for instance Table 2 of /29/ or /54/) and does not inhibit early “stages” (0, 1, 2). Conditions of their experiments were very favorable because the MCSF helps to the AZT action, especially in the macrophages /68, 67/ and the lower AZT dozes are needed for inhibition at the higher HIV infectivity with MCSF /68/ and the AZT is phosphorylated even better in the cells from AIDS patients /68/. A greater efficiency of even the AZT conjugate with low density lipoprotein /69/ confirms an essential role of the AZT intracellular transport.

§3. Intracystemal A Particules are the Artefacts of HIV Presence in Seropositives.

But how and why, in other experiments, do the authors find the infectious particles In asymptotic “phase” /58, 70/? In stimulating very insistently the cells with signal as MCSF or phytohemagglutinin, authors force an increase of the cell exocytosis (pinocytosis CIRCLE pathways) from cytoplasm area near endoplasmic reticulum by fundamental transport vesicular signal machinery (exactly resembling the extremal cases of the neuron growth cone or growing lamellipodium)/Zagyansky, retired Application FR-95-11550 with Refs./. One sees the same special universal way for basic fibroblast growth factor or Major excretion protein (Cathepsin L) /71/ or an increase of such vesicular transport reflected by increased vesicle fusions with plasma membrane after hormone applications /72, 73/. So, by this way, the gag coated pseudoparticles/justly, intracystemal A particles/(with RNA and reverse transcriptase but without env proteins) exit the cell from the cytoplasrnic vesicles and enter into other cells where the RNA can serve for a synthesis of separate HIV proteins that can be exocyted. Evidently, a mesurement of the p24 gag protein concentrations, the reverse transcriptase activity or polymerase chain reaction of exterior solution /65, 70/ produce a sense of a presence of the infectious viral particles although in very weak concentrations. But without mitogens and rigurous conditions of waitings, the virus “multiplications” were not found in similar conditions (see, for instance, /221).

§4. The Heterogeneity, Immunosuppression and nef Function.

Such special nonproductive HIV entry permits to make the important heterogeneity of env viral proteins absolutely necessary for later productive HIV contamination and also the particular immunosuppressions.

As it was proven /54/. the order of an appearance of antibodies to different HIV proteins could be judged from a type of antibody subclass due to its sensitivity to repeating of antigen stimulations and a quantity of antigen /29,Table 1,FIG. 1/. Last data confirm such thesis: an elevation of plasma IgE levels preceeds a decline of CD4 cell count /74/ and consequently, the high IgE level indicates a 100% rate of AIDS /75/. An appearance of a large quantity of new synthesized viral proteins at productive contaminations stimulate the IgE switch /54,29-FIG. 1/. Also the continuous spontaneous immunoglobulin secretion (throughout all stages of HIV disease from seroconversion) of cultured blood cells from patients shows that there is a continuous stimulation of these B-cells /76/ due to, justly, the present special anti-env (it means switching anti-MHC) antibodies /29, 54/.

But the new data with nef proteins permit to make very interesting conclusions. Logically, the nef proteins serve: (1) to make the heterogenous DNA copies and also (2) to disturb the HIV particles exocytosis and ,asynptomatic phase. There is no AIDS stage at using the attenuated SIV virus (deletion in nef) in vivo (evidently at high virus doze in cases of one injection- §6) /77-80/ with antiviral antibodies titre ˜10 times less /77/. This antibody titre increased in time (with new immunisations). /78 80/. A presence of such attenuated virus correlates, justly with absence of the immunodeficiency /78, 79/ at asymptomatic phase.Moreover only one strain is found at alive (during 14 years) blood donor (free from HIV related disease) with the damaged nef gene and a much lower number of the. HIV DNA copies is found at this donor and his blood receplents than normally during HIV. seropositive stage /81/. So one can estimate that the nef proteins influence the DNA reverse. transcriptase synthesis provoking the numerous heterologous DNA copies presence (normal course) where con-sequently a mixture of antl-env and anti-gag antibodies against corresponding (to numerous DNA copies) numerous proteins makes the Immunodeficiency The direct nef protein influence on the reverse transcriptase DNA synthesis /82/ confirms such conclusion. Other data confirm that the nef is connected with early stages of the virus replication /83/.

Justly such antibody PATTERN is NECESSARY to provoke the productive HIV entry with help of Fc receptors! This situation resembles one during 2nd contamination with wild dengo viruses by obligatory different virus subtype /54/: numerous antibodies molecules make stronger interactions with aggregated /84/ Fc receptors provoking the signal with productive. contaminations only in this case /54/. But at 2nd consecutive challenge of wild SIV virus (after introduction of the nef mutated strain) there is no AIDS stage, no increase of anti-env titre and no detectable wild virus presence /77/. This must happen. because the anti-env (restricted “mono” pattern) antibodies precipitate the new virus without virus entry!

It is a very exciting fact that the derived after 1st contamination sequences of HIV and SIV virus proteins have the changed threonines, prolines, lysines and arginines (as well Isoleucine) /85/ that signifies an alteration of the potential O-glycosylation sites confirming the corresponding rules by very independent way /86/. Justly, the potential N-glycosylation sites are also changed /85/. It must mean that a number of different (anti-sites) antibodies helping to the productive 2nd contamination attaches differently (by Fab fragments) to the elongated carbohydrate chains interacting by their. Fc fragments. with Fc receptors (that justly aggregate each other /84/).

However, the 1st, nef defective, SIV immunisation does not protect the infant monkey from 2nd challenge with wild clone /80/. This could happen because the infants have much more active macrophages /87/ that, exceptionally, can be contaminated productively with help of homogenous antibodies. During beginning. of AIDS stage, a quantity of anti-nef antibodies decreases /88, 89/ (although that of gag p24 increases) reflecting an absence (decrease) of the nef protein synthesis at this stage. Naturally, at such stage there must be a correct synthesis of justly productive homogenous /90/ particles with increased quantity of threonines and serines at envelope proteins /91/ with signalling O-chains.

But the nef proteins have also another action with help of their SH3-binding domain (N-part), making an association, for instance, with protein kinase-ζ/921 necessary for membranous cortical cytoskeleton contractions /Zagyansky Y. retired Application FR-95-11550 with Refs./ and, at simple special mutation in this nef domain, the SIV virus mutant (SlVpbj14) makes the cell transformation and, consequently, an immediate virus entry, multiplications and very rapid animal death /93/. But obviously, this is not the nef function because such simple mutation had to happen in nature leading to much more effective virus action in this case. But this is not a case and oppositely, at chronic contamination with this virus clone there is a loss of SlVpbj14 genotype during long term survival of macaques /94/. It must mean that another function of nef (after 1st contamination) is to attenuate signal machinery action in the negative interaction with key signal molecules in posessing the conserved proline-rich sequence (SH3 domain interacting) /95/.

Justly, (created with help of such attenuation) intracystemal A-particles (easily proteolysed) (§1) permit a more effective presentation of antigen to MHC-II molecules (it means more effective creation of antibodies) because the MHC-II presentation of endogenous secretable proteins is rmuch more efficient than that of exogenous proteins /96/. But endogenously synthesized membranous env proteins undergo the MHC-II restricted antigen processing only after expression on the cell surface /97/. It is an other confirmation that the anti-nef antibodies (soluble protein) appear as the earIlest /54/, and (as the anti-gag antibodies) they appear essentially earIler than the anti-env membranous antibodies—the key point of the course of the HIV infections in vivo /54/. Naturally, there is no influence of nef mutations. for HIV contamination in culture /98, 99/ because the mechanism in vitro is quite different /100/ and the antibodies are absolutely not Indispensable for such productive artificial contaminations.

§5. Clear Nongenetic Origin of Persistent Seronegativity.

As it was proved /54/, the repeated HIV virus contaminations can result in a long persistent seronegativity due to creation of the autoantibodies population after nonproductive virus entry. A clear correlation between the regular contacts of homosexuals and heterosexuals (including prostitutes) with the AIDS sick partners and the persistent serohegativity (in spite of clear traces of HIV contacts) /101-107/ confirms well such conclusion. Moreover, this mechanism /54/ explain clearly even the temporary seroconversion /104/ where some T- cells are developed before anti-gag p17 (anti-thymosin α) inhibition but later these cells became old and dead without new developed T-cells. The condom use with only partner part was (“strangely”) associated with a stronger seroconversion than no condom use /108, 109/ and the borrower of injecting equipment. most frequently appeared to have the lowest progression rate /110/. The confirmation of the general basis of complex HIV action /29-FIG. 1 ;54/ is clear. The repeated HIV virus injections in macaques could create the persistent seronegativity against env although anti-gag p55 (gag precursor) were present /111/.

There is the general nongenetic factors of the antl-env (HIV) product elimination due to only repetitive viral dozes (with some special time distances) for any (principally) person that conducts to the AIDS absence. Evidently, Independently, the genetic factors, that can also eliminate (influence) the anti-env production eliminate the AIDS stage (although It is not global factor, but the particular /as with special defective rare CCR5—2 allele Part. I, §4/).

The similar situation takes place with accord between the FcR machinery carbohydrate pattern and that of the HIV envelope, necessary for 2nd virus entry. The general nongenetic case is the AIDS absence at babies before 3 months old due to their, yet another, carbohydrate pattern for any infant (Part IV, §5). Also the 2nd AIDS stage absence at chimpanzees is the general case due to the quite different carbohydrate pattern of their FcR machinery (Part IV,§4). But also such situation takes place with the MHC class I alele Mamu-A26 (macaques) that correlates with general allotyping /112/ and different carbohydrate pattern (Part IX). The macaque FeR carbohydrate pattern (marked with Mamu-A26 allele) also must differ stronger from that of virus env, that conducts to their “protection” (in this very special kind of contaminations) /113/ (“immunity” of chimpanzees- Part IV, §4) at justly 2nd AIDS “stage” during entry with the FcR help. The (sorne) “increased frequency in human (long-term) survivor cohorts” of alleles, which resemble those of “immune” chimpanzees (seldom!) /114/ proves such conclusions. The same carbohydrate pattern disaccord of the FcR machinery with that of HIV env must take place at individuals with special alleles of HLA classes I and II and TAP (transporter) alleles, leading to (some) longer times of the AIDS survival /115, 116/.

A direct proof of nongenetic origin of the general persistent seronegativity is done with an activation of the peripheral blood mononuclear cells from corresponding patients in culture. In difference with short spontaneous secretion throughout all stages of HIV AIDS disease from seroconversion /76/, in this case, the long polyclonal activation, although only with added mitogen, must take place /107, 119, 120/ because these B-cells had, iustly, a time to pass the 1st stage of B-T cells Interactions /29, FIG. 1/ before the new immunosuppression by anti-Tα antibodies (due to 2nd antigen doze). Such polyclonal mitogen activation could remplace only the 2nd B-T cell interaction /29-FIG. 1, 54/ with, evidently, enriched quantity of anti-HIV B and T cells at 1st stage! Justly, an absence of such antibody systems in several cases of this persistent seronegativity /120/ shows difficultes of time coordinations for such events where a distance between the 100% life and 100% death is close /29,Table 1;54/.

Moreover, according to Ada “there is no convincing evidence to date for the SIV/monkey system that CTL are an important protective parameter“ and “such data is now accumulating from recent findings with HIV” /121/ (also /122/). The NK and CTL cells are effective only against cells with the 1st stage degenerated viral particles /121/ but they must be always very uneffective even against very small quantities of HIV, transmitted even by insects during 2nd contamination (already after proliferation of corresponding cytolytic cells)! /54/.

Part.IV. “AIDS: to the End. Futher Confirmations of General Strategy and Molecular Characteristics of AIDS Development.

§1. Mysteries of Epidemiology Well Fit the AIDS Strategy.

The epidemiological data also point out the necessary 2nd contaminations for AIDS development. A number of works have found the statistical correlations between the continued utilization of common seringe /1, 2/, a number of blood transfusions /3/ and a history of sex with someone in whom the AIDS developed /4, 5/ with a rate of AIDS progression from the seropositive asymptotic state. According to general course of the real AIDS disease 16-FIG. 1, 7/, the new HIV contamination justly must be a necessary factor for the disease progression.

But one artefact /6-Table 2, case No.4/ complicates this picture. The authors /4/ mistakenly classify, as the seronegativepatient, the case when there was already the almost immediate 2nd contamination leading to the case with obligatory low level anti-env (and possibly against other viral proteins) antibodies /7/ due to increased level of anti-gag p17 antibodies /anti-thymosine-(Tα) antibodies due to mimicry/ that blocks a development of T-cells /7/. A low level of antibodies against HIV was justly associated with subsequent AIDS /5/. Justly, this case had to be described as with low CD4 cell count “immediately” after seroconversion and an absence of a serological reponse to the HIV core protein (rather without memory IgG antibodies) /8/. This is confirmed by a presence of special anti-HIV IgM after such infections. Moreover, the “strangely” increased total plasma IgM level even before contamination /9/ points out that the recent. HIV 1st contamination already took place! The meadian life of mainly seropositive hemophiliacs. in USA has doubled last 10-15 years /10/, obviously due to decrease of 2nd contamination with improved blood transfusions.

So in cohort with really asymptotic seropositives (with anti-env antibodies) there is no AIDS disease development without new virus contamination that is confirmed with a cohort of seropositive drug users lived during 4.4 years in a monastery without drug consumptions and sexual relations /11/.

§2. Development of AIDS Course by Insects Only at 2nd Functional Contamination During Certain Blood Insect Mechanical Transmission of Any Virus.

It is easy to calculate that in region Belle Glade (Florida) (where a very important quantity of insects exists) a relation between quantities of the seropositives and AIDS-sick is ˜300 times less than in USA /7/. A progression from a seropositivity to AIDS is facilitated because much tower virus contaminations are needed for this transition and consequently this 2nd functional contamination could be done by insects /7/. This facilitated transmission is supported by the essentially lower speed of transmission (from fixed moment) from seronegativity to seroconversion than from seropositivity to AIDS /12/. This fact of a presence of numerous AIDS patients with justly pruritic papular eruptions (PPE) justly in Florida area (as well Africa or Halty, but not in other area of USA and Europe) /13, 14/ that must be due to insects (overpresent in South!) bites /7, 13-15/, confirms once more the BeIle Glade data. The eruptions are situated mainly on extremities (95% on legs and arms) (where insects could bite through or under thin clothlngs /15/) and “the morphology of acute lesion is characteristic of an insect bite” /13-15/.

The general fact of biological and mechanical transmission of many virus diseases is well established /16-18/ even for mechanical transmission by different insects (mosquitoes included) for 3 retroviruses /17, 18/. So a transmission of contaminated blood by insects is, too evidently, sure: evidently with transmission of ANY virus that the blood contain! A question is only about a possibility to overcome the immunity defense with a particular quantity of virus. Even a single horsefly can transmit the EIAV lentivirus disease /19/. A conservation of HIV virus in insects even until 8 days is shown /17, 18/.

The data of molecular and cellular biology confirm such epidemiologic data /7/. An insect bite with eruptions is the initial manifestation of AIDS stage (in such eruption cases!) 17, 13, 14/. The PPE were found only at patients with well decreased CD4 cell number /7, 20/. Moreover there are more precise cases when insects bite the seropositives where, as result, there is an appearance of POE /21, 22/ and relatively soon there is a death from opportunistic diseases /21/. After penetration of viral particles under a skin there must be the (firstly) occatlonai their meeting with anti-env antibodies and macrophages. As result, productions of new infectious particles as well CC chemokines take place /6/. Such similar CC chemokines attract the new macrophages and eosinophils /6, 7, 23/ to a bite area. With development of new viral particles there is a passage to the AIDS stage /7, 13, 14/ with increased quantities of IgE, characterizing justly the late stages /6, 14, 20, 24/ and a decrease of quantities of CD4 cells /6, 14/.

But at AIDS, the envelope proteins are shed into serum /25/ and they attach to cell surfaces /26/ and inhibit a migration of mononuclear and polynuciear lymphocytes and monocytes /27, 28/ and suppress an activity of cytolytic cells /29/. So there is a registed accumulation of eosindphiies (and macrophages) in such created eruptions /7/. The eosinophils secrete substances like TGF-β and a /30/, absolutely necessary for neighbouring cell divisions/Zagyansky, Y. retired Application No.FR-95-11550).

Evidently, at very -late stages of AIDS (high concentration of env proteins in serum, rather), such eruptions had not to be created because of strong depression of cell motility. An utilisation of zidovudine at late AIDS stage conducts to (generally new!) appearance of PPE after clearly insect bites /31/ due to some increase of cell motility with decrease of the env protein concentrations in serum. At some later stages of AIDS (at movement of monocytes and eosinophils not so suppressed 132/), an exagerated insect bite reaction can yet take place /33/ although it is not already decisive for AIDS mortal course.

Evidently, other parasites (as protozoans, yeasts, fungias) can also provoke similar eruptions (at beginning of AIDS) /34/ with mobile and immobilized later monocytes and eosinophils and one can suppose even the fatal contaminations (2nd stage) of seropositives at contamination by these parasites (with native virus) from the AIDS men. The very strong development of Kaposi's sarcoma at seropositive patients contaminated with Mycoplasma /35/, that is the cofactor of AIDS development /36/, confirms such claim.

§3. Opportunist Infections: Facilitation of 2nd Contamination at AIDS Stage by Complement Receptor Due to Activation of C1q Fragment.

The opportunist agents includihg heroes viruses and tubercle bacillus accelerate the death due to AIDS stage /37-41/ without real change of a spread of transition from asymptotic phase to AIDS /37-41/ (/41/—with analysis of errors of other works). It is shown that the polycional anti-env antibodies do not activate the complement /7/ but the complement (C1q!) activation by other antibodies (against opportunist antigens) can lead to attachment of this activated C1q to anti-env antibodies together with attachment of C1q itself to its special C1q receptor on the HIV attached cell that facilitates the Fc (once morel) receptor mediated HIV virus entry /42/. A reality of the signal between C1q and its receptor (C1qR) and C1qR binding protein /43/ and a special synthesis of C1q factor by macrophages /44/ confirms such conclusion. An elevations of the IgE concentrations, associated with opportunistic infections /45/, reflect an increase of virus antigen concentrations leading to IgE switch /6,FIG. 1/. A similar facilitation happens with C3 fragment and CR2 receptor /45a/.

§4. “Immunity” of Chimpanzees as Weak Correspondence Between Fc Chimpanzee Cell Machinery and HIV-1 Patterns of Carbohydrates.

The strange “natural” protection of chimpanzees against HIV-1 is easily explainable. The similar carbohydrates pattern of CD4 and other suitable molecules /48/ must permit the 1st contamination of chimpanzees in vivo /47, 48/ with resulting heterogenous HIV proteins pattern (49/. But some different chimpanzee carbohydrate pattern of its Fc receptor machinery (for active HIV-1 virus particle production with help of anti-env antibodies), logically, does not correspond well to HlV-1 carbohydrate pattern and the productive contamination with AIDS is absent /47, 48, 50/. The complete lysis of T helper chimpanzee cells by HIV-1 virus (within 3 weaks) in vitro /50/ confirms such conclusion. So in reality, such “natural immunity” (at 2nd contamination) is posessed by all other animals, only in the case of the closer (to human beings) chimpanzee a similarity of carbohydrate pattern generally exceptionally permits the 1st contamination.

§5. Child's AIDS Adapted to Particular Characteristics: Active Immune System, Mother's Antibodies and Change of Fc Receptor Carbohydrate Pattern Around 3 Months of Age.

The resolved already important problems permit to resolve sufficiently complex case of neonatal AIDS with a presence of mother anti-HIV antibodies and discrete change of the carbohydrate pattern at lc receptor complex around 3 months of age.

In a number oqf cases there is a viral contamination of neonatals before or during birth /51/. These special contaminations of (mainly) macrophages (within 60 days of neonatal life /52, 53/) must lead to a synthesis of viral proteins and intracystemal A-particles (IAP) appearance /55/ that leads to the heterogenous antibody production /53, 54, 56/ although the mother antibodies must make some inhibition of antibody production /57/ blocking different epitopes on HIV chains during antigen presentation /58/. The healthy neonatal immune system can already early produce the antibodies /59, 60/. Moreover, the neonatal macrophages are more (!) active /61/ that must be important in contaminations also. The positive polymerase chain reaction (PCR) test at 1 month predicts the antibody production presence (98.9%), revealed at 15th month /56/. Justly, as in the case of adult contamination at higher viral dozes /6/ there is the encephalitis development (depending, evidently, on AIDS mother status) /62, 63/. The CD4 cell quantity of such infants can diminish in 185 times at 9th month /63/. In difference with the 1st adult contamination, the cytolytic cell accumulation is weak (before infant antibody production) /64, 65/, logically because the mother antibodies block the HIV chain epitope interactions /57, 58/. The SIV virus injection (strong dozes) into neonatals Induces the viremia (antigenemia in reality) and the antibody production is difficultly mesurable but after the zidovudine treatment, the viremia (antigenemia) diminishes and the (mesurable!) anti-SIV antibody level increases /66/, logically, due to diminished antibody precipitation.

From the beginning there is the immunosuppression with the mother antibodies /67, 68/. Such immunosuppression must exist during the embryogenesis of each embryo of the seropositive mother and the embryo thymus has the pathological abnormalities /69/ due to, logically, the anti-T(α) (mimicry with HIV gag p17) antibodies /71. Due to such immunosuppression in HIV infected children (logically seropositive) there is the weak answer to the influenza virus vaccination /67/. A number of AIDS associated diseases /68/ and increased quantity of the lethal cases (not AIDS-linked, although anti-HIV antibodielinked) /68, 70/ must be due to the immunosuppression of the unexperimented infant immune system due to the mother and (later) infant mimicry anti-host (against important molecules participating in immune system action) antibodies /7/. Such diseases correlate with mother CD4 counts at birth /72/ because of a higher level mother anti-HIV antibodies and a higher level (possible) viral perinatal contaminations inducing a higher heterogenous antibody answer.

In the case of the infant AIDS there is a convergent confirmation of the law of homologous intercarbohydrate interactions. As I mentioned, the Infant immune system is normal and the macrophages are even more active. But a number of events take place not from beginning but (all) very insistently from 3-4 months: (1) CD4 count decrease (with threshold for AIDS course) /68, 73/; (2) AIDS diagnosis (CD4 count decrease) at blood transfusion /73/; (3) mortality of the strongly contaminated with SIV newborn monkey /68/; (4) process of mother anti-HIV antibody disapearance in the case of AIDS /74/; (5) “sudden” new appearance of IgM infant antibodies /75/.

So, what is happen at 3-4 months with good already infant immune system? Justly, the carbohydrate pattern changes with ontogenesis including a period after birth /76, 77/ where the IgG oligosaccharides are indispensable for interaction with Fc receptors /78-80/ (making important self-aggregation /81/) and where justly the macrophage Fc receptors are important for productive entry of HIV virus in vivo /6, 7/. So, logically, the new carbohydrate pattern of Fc receptor of infant accords with that of HIV envelope proteins from 3-4 months and the productive AIDS course can begin ONLY from this time. The “strange” new seronegativity of infant from also 3 months /82/ can be well explained with intensive creation of a number of new infective viral particles that are precipitated by anti-HIV antibodies. The new (once more) seropositivity at ˜8th month /82/ must happen due to creation of the anti-viral infant antibodies. A better HIV productivity in vitro of neonatal (at birth) than adult macrophages /61/ confirms a help of antibodies with Fc receptors from 3-4 months In vivo.

Because there was no (in past) comprehensively analysed correlations of viral burden p24 antigen, CD4 cell count course /83/, precise breastfeeding data, parasites presence (§2) and titres of mother and infant antibodies, I present the scheme of consecutive events in such complex situation. Without creation of anti-HIV infant antibodies after the birth until M3ronths, the anti-env heterogenous mother antibodies will help to create the virus production after ˜3 months. An interplay between depletion of heterogenous mother antibodies (precipitation), a development of heterogenous infant antibodies (or may be individual success of homologous infant antibodies /54/) and a presence of yet active viral particles would decide a child's destiny: a life with long seropositivity or AIDS with death.

But how do these necessary 2nd type contaminations happen? I concluded before that the AIDS, with obligatory 2nd contamination, must generally take place with breastfeedings /7/. Such contaminations with breastfeedings were clearly proved where the postpartum newborn seroconversion was associated with the high risk contaminated milk /84-87/. Moreover, it follows that without breastfeedings with contaminated milk, the AIDS stage does not occur /7/. An absence of seropositivity against other immunodefficiency virus at goats without feeding with contaminated milk /88/ and the general decrease (at seropositive mothers) of the “mean” transmission rate in two times in the case of absence of breastfeedings (in comparison with bottle-fed infants) /89/ confirm such judgment. However in the case of HIV infected infants, the deaths happen even in the case of absence of breastfeedings (in French cohort) /82/ because of the weakened suppressed infant (but many specific antibodies against different diseases) immune system. A reality of such deaths is confirmed with a help of mesurement of CD4 cell counts (threshold 1500 cell/μl from 3-6 months) evolution /68, 90/.

There are 2 types of patterns of infected children: long and short term survival. The short term survivers belong justly to described cases with rapidly lowed CD4 cell counts /91/ although the CD4 cell kinetic must be mesured for each case. Long term survivers are the seroposltives that could be contaminated the 2nd time already later with very small dozes /1, 7/ due to different reasons as home parasites (§2) or absence of hygiene, especially in living with AIDS-sick parents that must be very dangerous for such children.

§6. Principal Schema of HIV Infection (In Vivo).

At the end of this development of principal bases of HIV strategy, understandable only with comprehensive molecular biology, I present the general principal schema of HIV infection (in vivo).

One can understand that the cell type influences the viral carbohydrate pattern which clearly depends on the type of cell /79/. And a similarity of this pattern to that of the cell receptors, glycolipids and proteoglycans will determine the infectivity with the best necessary melting of the plasma membrane /6/. This miust be the molecular basis of the viral and bacterial virulency.

The principal targets of the 1st HIV contamination are the moving macrophages (monocytes) /e/. There is a presence of contamination during its early phase in macrophages but not in lymphocytes /92/ with help of the macrophage-tropic (it must mean: issued from macrophages with their best /93/ carbohydrate pattern) variants /94/. This nonproductive contamination creates antibodies, very Important for the next stage, with so called, nonsyncytium induced isolates at asymptotic individuals /95/. At the 2nd contamination, the new macrophage-tropic variants contaminate productively the new macrophages with help of the anti-env antibodies /7/. The clear proof of such macrophage target at 2nd contamination, one can see from the established and quite independent epidemiological data. The seropositives with pneumonia “strangely” have a decreased AIDS development /96, 97/. And justly the Pneumonia bacteria turn of the macrophages making from them the multinuclear syncytia /98, 99/.

The new created infectious viral particles (with some “old” ones?) contaminate (with antibodies) the T4 cells. Such lymphocytes undergo the apoptosis and this “apoptosis was tight associated with formation of syncytia” /100-102/. A change from the non-syncytium Induced to syncytium-induced isolants correlates with AIDS progression /103/. Only the macrophages but not the T4-cells make the budding (in vivo!) of new viral particles to exterior /103/ and the viral particles remain preferentially In cytoplasmic vesicles of T4-cells /103, 104/, especially prived of the signal transport machinery. The macrophages avidly phagocytize justly cells during apoptose /101/, it means the contaminated T4-cellsare regularly destructed. So the quantity of the contaminated T-cells, present at the moment (of death Included), must be low but a number of T4 cells diminish sensibly /106/ although it makes a time (˜7-20 months) from 2nd contamination until death /107/ justly in accord with this mechanism (FIG. 3). A contamination of some small quantity of lymphocytes at 1st stage is possible, principally, especially with the T-tropic variants /108/. Logically, a binding of chemokine lymphocyte CXCR4 receptors with their natural ligands (SDF-1) slows the progression of AIDS and “blocks the virus entering the T-cells” /109/ blocking the syncytium creation(FIG. 3).

Part.V. “AIDS: to the End. Clarification of Vaccinations: Solutions and Impasses. Generalisations for Virology and Simply Makable Experimental Confirmations”.

§1. Clear Principal Utopy of Actual, Strategies of Vaccinations Against HIV. Charged Anti-Envelope (env) Antibodies and Specific Elimination of Clones of Anti-Env Antibodies as Effective Means Against AIDS.

A very voluminous literature concerning vaccinations against HIV presents very heterogenous and very difficult unresolved problem today. But with resolved here and before /1-4/ fundamental bases of AIDS development, the problem can be clarified with a good agreement between such very different data and, unfortunately, with understanding of the obvious impasse of existing anti-HIV vaccination strategies.

As it was pointed out, the vaccines against HIV without native nef proteins eliminate a creation of the heterogenous viral (and antiviral) population at 1st stage with general impossibility of the stable 2nd AIDS stage that takes place only with heterogenous antibodies /2/. But with challenge of the wild virus there is only some delay/due to homogenous (only precipitating) anti-env antibodies/ for heterogenous (1st stage) anti-HIV antibody production that cannot stop, in reality, an established course of AIDS /1, 3/. As it is also shown, the natural “immunity” against HIV of chimpanzee is the same as for any other animal or bird, only, In difference, the evolution closeness of chimpanzee permits,however, the 1st stage /4/. It means, the vaccinations of chimpanzee /5/ serve clearly to prevent this 1st (and last, already) stage.

Normally, according to proven mechanism /1/, an injection of homologous retrovirus into seropositive animals, generally, must lead to severe immunodeficiency illness (2nd step) /6, 7/. It must happen due to enhancing effect of antibodies (with help of Fc receptors) which “contribute to the spread and pathogenesis of HIV in vivo” with time of illness tested partially with corresponding patient serums and patient virus isolates /8/. In reallty, after Interaction of the native virus from 2nd contamination with heterogenous antibodies (from 1st nonproductive but necessary contamination), the AIDS development must take place.

Generally, a protection was conceivably done only when the vaccine and challenge virus were obtained in heterologous cells (as a protection against SIV virus at monkey with vaccine and challenge virus grown in human cells but “strangely” not in homologous monkey cells /9/). An additional difference between carbohydrate pattern of SIV envelope proteins in human cells and that in monkey cells must make an additional problem for virus entry. Obviously, it is unacceptable as vaccination.

Generally, the antibodies against inactivated virus or Its env proteins must be more homogenous although passages of virus (taken from sick patient or animal) through the unnatural cell cultures must make such virus more heterogenous /10/ (with unpredictable degree) and these antibodies make a delay of 1st stage entry of challenge homologous virus /9, 11, 12/ that must depend on conditions of a production of the virus for immunization. As result of such (however) entry, the heterogenous antibody production takes place (after heterogenous viral proteins synthesis) /2/. But at the same time, these yet homologous antibodies can, however, make some visible restricted sporadical entries into cell with help of Fc receptors /1, 3/ that one can see, for instance, in clear variations of CD4 cell counts in seropositive phase in some cases /9, 13/. There are the reasons to considerate that the immune response after SIV vaccination does not correlate with protection /14/. Such, even sporadic, productive entry with help of the vaccine produced homologous antibodies already proves the clearly definitive unperspectivity of such vaccines. When the heterogenous antibodies appear before the native virus elimination (in such interplays), the AIDS stage takes place /7-FIG. 1, 11-FIG.1/.

A potential subunit (env) vaccine to enhance the AIDS disease /12/ can be due to high concentration of antiglycoprotein antibodies and their some heterogeneity due to passages of original virus through culture (see also /15, 10/) where such massive presence of some heterogenous anti-env antibodies helps for (at once) productive virus entry at 2nd stage as well for increased polyclonal production of anti-HIV heterogenous antibodies of 1st stage /1, 3/. An enhancement of the FIV infectivity with Immunization by the recombinant env of FIV (produced in nonhomologous hamster cells) before the homologous FIV challenge /17, 18/ could be rather due to increased polycional production because of anti-env (anti-MHC-II also) antibodies /1/ produced generally at 1st stage with heterogenous antibodies .

The case with passively transferred (at high doze) anti-SIV antibodies from seropositives /11/ well resembles the complex case with mother's antibodies in baby after 3d month of age /4/ where an Interplay between diminished ist virus entry with appearance of the heterogenous stable host antibodies and appearance of new active viral particles (2nd type of entry) with decreasing of concentrations of the transferred (or mother's) antibodies must decide a destiny between a death from AIDS or a life, as in the case of contaminated baby /4/.

The solution for AIDS is coming by the way of a profound development of molecular cellular biology. It was proved principally new conception /9/: a beginning of the signalling takes place at the outside surface of cell with the key-positions of the intercarbohydrate and interlipid H-bonds interconnected in the special glycoprotein-gangliosides-cerebrosides-proteoglycans- . . . complexes. Consequently one can destroy the cell signalling already from outside, for instance, in perturbing such essential network of H-bonds with strong charges /19/. In labeling the specific antibodies (or lectins) against viruses (that must switch the signalling /3, 10, 19/) with strong charges (or producing them with help of bioengineering), one can effectively neutralize any such virus (or bacterium), HIV comprized /19/. Also, one can eliminate specifically the clones of anti-env (or ANY as causing the rheumatism) (it means: corresponding B and T cells) with help of charged antibodies against active site of such antibodies /1/. Without the anti-env antibodies, the 2nd contamination and AIDS have not to be happen /1, 3/ as in the case of inactive β-chemokine receptors with corresponding absence of seropositivity /3/. Excitingly, about 50 years ago, the very intensive (almost forgotten) complex of works with very intensively charged polymers did claimed an end of viral diseases /Refs.20/, but nonspecificity of such preparations had to kill also the host. Moreover, the considered action “mechanism” of such polymers was incorrect, principally. So in the scientific darkness of only some empiric reflections of real remedies, they completely abandoned such so intensive works. But recently, the new series of works with negative charges (as heparin or succinylated albumin) against HIV virus begun /21, 22/. The similar nonspecificity (strongly limiting necessary concentrations in vivo) and, evidently, nocivity cannot be accomplished with real remedies against HIV,however the essays to approach to above effective, clear in purpose and mechanism, global strategy take place.

In confirming convergence of mechanism of HIV env gp120 signaling with the O-chains intercarbohydrate interactions at V3 region /10/ and the above mechanism of disruption of the essential network of the intercarbohydrate bonds with heavy charges /19/, it is “strongly suggest(ed) that the heparin exerts its anti-HIV-1 activity by binding to the V3 loop of gp120” /23, 24/.

§2. Artificial Conditions of HIV Contaminations In Vitro.

From these data /1-4, 10/, it is evident that the nowdays conditions of the cellular HIV contaminations in vitro are artificial and do not correspond principally to the real contaminations in vivo. The first HIV contamination, with the concentration gradients and corresponding changable substrates /3/, was never reported in vitro.

So in compensation of the above favorite conditions in vivo, the high, unnatural, virus concentrations, moreover with special artificial signal activating ligands /1/, are used where as result there are the two types of viral entry: (1) by endocytosis and (2) by direct fusion /3/. Justly the virus endocytosis characterizes sufficiently strong concentrations where the patches of only VIRAL exterior molecules can induce the endocytosis /25/ with nonproductive contamination /1/. At greater concentrations, the created aggregates of virus particles /28/, attached near the cell surface, must create a great number of interviral intercarbohydrate homologous bonds with the powerful local dehydration and membrane destabilization /3/ for direct fusion, as in the case of the syncytium creation or the cell-cell fusion with a help of viruses or polyols. So “the syncytium formation is often the first sign of HIV infection in culture” /27/. And there is a total (In difference to small “problematic” quantities of the found artefacts /28-30/) infection of cell as fibroblasts that is not found in vivo /28-31/ and the CM4 receptor does not participate in such global artificial infections /32-34/.

Moreover, the good HIV particle production in vitro in the case of newborns which cannot take place In vivo at this time /86/, the good HIV particle production in vitro in the case of chimpanzees cells with an absence of such production for the AIDS stage in chimpanzees in vivo /3/ and an absence of an influence of the nef-mutation in vitro whereas such mutations clearly eliminate the productive AIDS contamination in vivo /3/ prove once more that a mechanism of viral contamination in cell culture is quite different and cannot be directly used for AIDS mechanism interpretation in vivo (anyway in all yet such usual conditions). Showing an Increase of infectivity after utilisation of the anti-HIV serum antibodies /1, 27/ (2nd contamination), the authors do not decrease virus concentrations until the level In vivo, where this quantitative effect must be transformed in qualitative: absence of effective contamination against its presence. And classically, these means are not used for culture contaminations or determinations of virus titres.

§3. Unnaturally Large Dozes Lead to Dispensibility of 2nd Virus Introduction. Confirmation of General Strategy.

But these two types of HIV contamination in vivo can take place during only one HIV introduction. Evidently, there is a limit of a capacity of the inmnune defence (1st stage) and one strong lentivirus contamination must kill already the victim. With an increase (ARTIFICIAL CONDITIONS) of infectious viral concentrations, the antl-env antibodies appear before the complete elimination of introduced active virus particles after 1st contamination, so the death due to the AIDS after one virus introduction confirms this schema /4-FIG. 1/. The experiments confirm such consequence /35-38/ according to which the virus is normally present during the antibody appearance although capacities of organism immune system are different (logically at the limit of the lethal dozes) /36/ which depend also on the way of virus Introduction /37/. One peak of viral presence until death or one peak with another one (2nd) (2nd entry type with antibodies) beginning before descent of the 1st peak until zero (or Immediately after 1st peak with, logically, undetectable by method, small viral particle concentration) (as /38-FIG. 1 A,B/) characterize the one large doze injection with the AIDS progression.

§4. Lower Variability of 1st Stage and Worse Fit of the Carbohydrate Pattern Must be Responsable for Restricted HIV-2 Contaminations.

From above data, the causes of biological and structural differences between HIV-1 and HIV-2, leading to the restricted HIV-2 contaminations, can be easily concluded. Logically, the carbohydrate pattern of HIV-2 fits less to such CD4 molecules that makes a worse affinity of HIV-2 to CD4 and more difficult entry at contamination (1st stage) /39/ where as result there is much less geographic area of contaminations with HIV-2 in spite (even) of certain expositions to virus /40/. Some regional difference between the carbohydrate patterns of populations is evident.

Moreover, a reason of prolonged periods of the asymptotic seropositivity at the HIV-2 contamination /39,41/ can be explained with lower variability of amino acids (ist stage) of gp120 protein of HIV-2 /39/, necessary for 2nd HIV entry with help of the Fc receptor /2/. A better protection of HIV-2 infected women than of seropositive ones against the HIV-1 contamination /39, 41/ must be due to a partial block of some (necessary for HIV-1 infection) cellular (carbohydrate) epitopes with anti-HIV-2 antibodies.

§5. Generalisations for Virology.

One can suppose that such effective HIV virus properties can be used by other viruses. Really, for instance, the quite different DNA viruses (as CMV, EBV or HPV) have a number of properties that resemble these of HIV virus in particular and lentiviruses in general. It was already shown that CMV and EBV viruses also have the acute and latent phase with help of heterogenous antibodies against the synthesized viral proteins (that could be included in pseudoparticles) (§2) that can also provoke the immunosuppression /42/. At also 2nd phase with new contaminations, in a presence of anti-viral (env) antibodies there is the more effective entry of herpes viruses with more severe recurrent diseases /43-45/ (§2) although (In difference with HIV) there is no integration of DNA in such cases. Logically, the severity of above recurrent diseases must be again amplified with signalings as, for instance, that appeared during transplantation /43,46/. But such integration can happen spontaneously /47,48/ or with AIDS development.

It is natural that other viruses use the cell movement for entry (1st “latent” entry) at low concentrations and a number of viruses code the G protein-coupled receptors (GCR) resembling different chemotactic receptors /49-51/ where such receptors could be situated even in enveloped viral particles /51/. So the migration increases at contaminated cells /52/ (and also through the own classical cytokines as IFN-γ, TNF-α, IL-1 /53/ ).

In the case of the well studied CMV, cell receptors for the virus attachment are common in a number of cell types /54/ but the monocytes/macrophages are again (as with HIV) the key elements in the acute and latent infection which once more also have a low percentage of the contaminated cells /55/. The same “Trojan horse” model is valid for CMV too /77/. And markedly increased quantity of attracting chemokines are synthesized in the contaminated macrophages situated in target organs /52,56/. The CMV β-chemokine-like receptor UL 33 (M 33) appears justly at a beginning of a contamination (naturally justly during migration), confirming such data. Without this receptor, the viral growth strongly diminishes in salivary glands. However, in vitro this receptor does not influence a growth /51,57/. This fact, once more, clearly confirms that conditions for virus contamination in vitro are taken artificially /1/. A number of other CMV genes are not essential In vitro neither but they are active in vivo /57/. A lack, in vitro, of the model for a latency /80/ confirms such conclusion. As in the case of HIV In vitro, the endocyted enveloped CMV must be in large intracellular organelle although (also) because of a penetration of the virus “only” due to its increased concentrations /1, 10/ there is a presence also of the direct fusion /59/.

A number of other 13-herpesviruses makes also the homologous of the chemokines /50/ that evidently also-serves for a stimulation of the directed cell movement /60/ facilitating the virus entry by endocytosis with the consequent latent state as the obligatory stage for each herpesvirus /61/. The γ-herpesviruses also stably produce such homologues /50/. Also, for instance, the EBV (Epstein-Barr virus) upregulates the expression of the two α-chemokine cell receptors in B-cells but not in T-cells /12/ and, justly, the B-cells (naturally by endocytosis at trafic) but not the T-cells are contaminated /85,p.2345/. A presence of capping at the primary B-cells after interactions with high (well visible) viral concentrations /64/ confirms well such data. Also the IFN-γ and IL-1α increases strongly during such phase /65/ and IFN-β inhibits the capping /64/. Naturally, without knowledge of a precise mechanism for contamination in vivo,contamjnations in vitro (always at large concentrationsi) usually have not to use a cellular motility at low viral concentrations and globally, because of this, there is no real latent stage in vitro for one well studied β-herpesvirus /58/. For exemple.

Such produced chemokine receptors are justly absent at all α-herpesviruses /50/ and, for example, the 1st entry of HSV (human simplex virus) must take place differently, by direct fusion /66/, that justly cannot be done during the well synchronized cell motility but after the direct attachment to corresponding cell surface receptors. Such, even direct fusion, In this case, does not promote the signal sufficient for a productive infection but leads once more to the latent infection /60,87/ and consequently the Universal signal NF-kB complexes could be detected in nucleus only in 8 hours /18/ reflecting very weak signal with an activation of the IFN-α,β mainly /89, 70/ where the type I IFN (α, β) conducts to a synthesis of the anomalous viral particles and the type II IFN (γ) to a reduction of their release /70,71/ and one can observe that this is a more general property of the above interferons against a number of viruses (as for example SIV or HIV /72/). A reason of such weak signal at the direct fusion is following. The viral envelope glycoprotein reacts with Universally present heparin sulfate proteoglycan (HSPG) molecules /73, 74/ (potentially with syndecan /75/) with oligomerization /73/, and the particular gangliosides are also necessary for HSV action /76/. A number of microfilaments must attach to such massive syndecan aggregates /3/ making disequilibrium and as result, the signals as the spreading and migration are inhibited in contaminated epithelial cells /77/. As proof of the signal weakness, a reactivation of the latent HSV infection in neurons can be done after neuroctomy /78/ which activates well the signal machinery axonal transports.

The antibody-dependent enhancement of infection, the key point of this work (detailly explained here) is relatively spread effect that takes place with quite different virus FamiIles: Flaviviruses (dengue, yellow fever, japanese encephalitis, langat encephalitis, St Louis encephalitis), Coronaviruses (feline infectous peritonitis virus), Orthomyxoviruses (influenza A), Lactate dehydrogenase elevating virus and related viruses (PRRSV virus), Parvoviruses (aleutian mink disease parvovirus), Paramyxoviruses (respiratory syncytial virus), Togaviruses (venezuellan equine encephalitis), Picornaviruses (foot-and-mouse disease virus) /79-82/. A majority of them are coated viruses but there are also uncoated viruses. Moreover, they include the DNA as well the RNA different viruses.

So consequently, one can see, even from these fact examples, that the discovered profound.properties of HIV lentiviruses must be much more general and even could be revolutionary in all virology. For instance, ununderstandable variations of viral vaccinations since Pasteur (until the very grave effects) /79/, logically, must be a consequence of a homogeneity or heterogeneity of antiviral antibodies /2/. Consequently, all antiviral vaccines and antiserums must be urgently tested for such characteristics. For instance, the passages of the virus for vaccine through diferent heterologous cells or a high titer of antiviral antibodies after vaccination must be dangerous for a number of viral famiIles.

§6.Clearly and Simply Makable Definitive Experimental Confirmnations. “Troyan Man Model”.

So the excellent convergence of different conclusions presents the whole concised uncontradictable picture of AIDS development based moreover on the latest developments in cellular and molecular biology. I do not see any serious contradictions. with all established data concerning the AIDS development. Moreover, already the machanisin of different very important herpes viruses (CMV, EBV, HSV) and the vaccine development for many viruses confirm that a number of developed here (absolutely new) conceptions must be very important for All Virology.

A number of fundamental definitive confirmations can be done even during very short time: (1) easy clarification of a presence of IAP (intradysternal A-particles) in real seropositives after their creation /2/ but not of the, HIV infectious viral particles: end of a myth of sleaping lentivirus; (2) creation of conditions of real AIDS development in vitro: a)lst contamination: chemokine and cytokine concentration gradients for macrophage movement with corresponding (to in vivo) substrates, b) 2nd contamination: low viral concentrations (their diapason without productive contaminations of sole virus in culture) with heterogenous antibodies from seropositives; (3) confirmations that each real seropositivity /4/ develops the AIDS stage only after a contact with AIDS sick persons (counting for parasite transfections with cutaneous signs-/4/); (4) . confirmation of AIDS contaminations by SIV of seropositive monkey by. AIDS. sick monkey with help of insects (and other parasites) and impossibility of such transmissions to seronegative monkey; (5) certain persistent presence of anti-gag p17 antibodies at persistently seronegatives /2/; (6) repeating of successful “monastery experiment” /4/ and immediate separation of seropositive children from AIDS sick parents (elimination of so called long children AIDS course) /4/. All these definitive experiments could be done 9 years ago with effective understanding and cury of AIDS /1/. Such special phenomenon, I name “Troyan man model”.

At the same time since 11 years, the imaginative theory with “tendency to mislead the literature” that is “not based on facts” /83/ and does not “applied to unrevelling the enjimatic mechanism of HIV pathogenesis of AIDS” /84/ fulfils pages of international level journals. Moreover, the WELL correct CRITIC of a number of contradictions in accepted today mechanism of AIDS development /85/ but from such “irresponsable (and pernicious)” (according to D.Baltimore and R.Gallo) author is taken in fact to confirm . . . “notwithstanding” argumentation /86/ for a little fantastic things that consequently-served generally to ignorate such his partially perfectly correct critic until today, in reality.

However, it is already 2nd case of “Troyan man model” since 1987 when after explosion of SuperNova 1987A, the neutrinos and antineutrinos arrived to Earth ˜18 hours before the light after the same explosion: e⁻+e⁺→ν+\overline{ν}+hν /87/. The proposed explanation about light delayed scattering has no sense at all because the time of delay for one scattering is ˜10⁻¹⁵ sec and even for 1 second of delay, it needs already 10¹⁵ cbllisions. So the experimentally discovered speed more than the light speed with the clearest difficulties for existence of Einstein's theory /87/ was omitted selon “Troyan man model”. The definitive expreriments must close these “Troyan men models” phenomena, especially “with projections of up to 40 millions by year 2000” of HIV infected adults /88/.

P.S. Moreover, such powerful advance (together .with principal resolutions of cell cycle mechanisms and cancer /“itself/” FR-95-11550, pp.1-77) permitted me already to resolve the clear causes of an appearance of the-turnotrsd at the AIDS (Y.Z. FR-98-03204), but this last part, I do not reproduce here because of my situation.

Part.VI. Practical Consequences of Parts I-V.

The discovery of the absolutely new and profound properties of HIV action (contamination during the 1st phase by relatively weak concentrations of virus, exclusively by mobile macrophages, the indispensable heterogeneity of the viral proteins, synthesized during 1st phase to execute 2nd contamination, the new type of productive contamination, avec antiviral antibodies, during 2nd phase of HIV action to make AIDS) revealed the fundamental proprieties of other types of viruses with practical applications in All Virology (Part.V,§5). Consequently, one easily eliminates the grave unpredictable effects during the vaccinations since Pasteur against such spread viruses as Influenza A (influenza/pneumania) (see, Part.V,§5). For this, one must utilize (for immunisation) the surface viral proteins with (preferably) one sole epitope (neutralizing but not increasing infection), received from one viral homogenous clone. The Revolution in Vaccinations is evident. One can easily find the conditions of vaccinations and of preparation of monoclonal and polyclonal antibodies in Grand Test Books /1-3/ and one can find a number of monoclonal (and polyclonal) antibodies /4/ that are neutralizing but do not increase an infection.

The encephalites, produced by a number of viruses (like HIV virus- Part.III, §1), have place due, at the end, the movement of the macrophages into the brain, conducted by concentration gradient of β-chemokiries (Part.I,§§1, 4). Consequently, in perturbing the movjrment of the macrophages (for instance, with antibodies against chemokine receptors), one prevents the encephalites, provoked by different viruses as CMV, for instance.

Evidently, titers of the viruses, determined in vitro, are false (Part.V, §2). The conditions for determinations of the virus titers must be the most close to ones in vivo. Consequently, even for sole HIV virus, there are the two types of determination of titers: during 1st and during 2nd phase. For nonproductive contaminations of viruses in vitro, resembling the 1st phase of the development in vivo, one must have the concentration gradient of the corresponding-chemokines where the motility, evidently, facilitates the virus entrance at weaker concentrations. One determines a quantity of exocyted particles at particular concentrations, for instance. In order the mechanisms of the productive contaminations resemble the 2nd phase of AIDS development (VIH), one must have the heterogenous antibodies against the envelope (imitiating the “spectra” of the antibodies of seropositives). Principally, one determines, here, the quantity of active viral particles.

Evidently, the HIV envelope proteins switch the complete signal to enter the cell /Parts I,V;Refs.57—Part VIII/including the creation on the cell surface of the network of the very essential hydrogen bonds (including those between env and CD4 carbohydrates) which can be destroyed (from outside) by the strong charges, locally introduced with help of the antibodies (lectins), directed against the env proteins, thus preventing the virus action. So one can make such effective antiviral preparations also against different viruses and also other parasites as the bacteria or machrooms, in making the charged antibodies against the molecules of their surface cutting their life (signallings). Also one can make such effective preparations with absolutely new principle, against the well distinct carbohydrate chains of the cancerous cells /RefS.7, Part VIII/in eliminating effectively and selectively the cancer! The classical trivial antibody preparations, one can see, for instance, in /1-3/ and the classical preparations of the cancerous cell oligosaccharides (antigens), one can see, for instance in Reviews /numerous Refs. in 7, Part VII/. By other means, one can eliminate,with such charged antibodies, the IB and/or T cell clones, producing the antibodies against the env HIV proteins, that also stops the AIDS /Part I/. Evidently, the same means (with elimination of the harmful antibody clones) can be used against all other viruses with similar two step action mechanism, wherein the own anti-env antibodies help to kill (to struggle) the host at the 2nd phase and against the harmful autoantibodies. in autoimmune rheumatic disease /5/.

The preparations of the “substantial number” of the different well charged polymers as poly (5-nitro-2-furaidehydeacryloylhydrazone); poly (D,L-lysine or arginine); polyaldehyde with aminosialicylic acid; copolymers of sulfapyridine, suffabenzarnide, sulfanilamine and 4,4′- diaminodiphenylsulfone; copolymers of sulfonamide and formaldehyde; poly (β-(5-nitro-2-furyl)acrylamine); poly (vinylamine); poly (L-α,γ-diaminobutiric acid); anionic polysulfates and polyphosphates; poly (N-vinylpyrolidone); effective oxydized. oxypolysaccharides (obtained easily as chlorite oxidi zed oxypolysaccharides) are well described /223 (!) Refs. from 20 of Part V/.

The coupling of these well charged polymers (with different lengths) with proteins (antibodies, lectins) could be very easily done, for instance, with the carboxyl and amino group activations, with the N-acyl activated derivatlves,with the famous coupling reagents with the help of the well described chemical properties of the chemically active reactive protein structures /Reviews 6-9/. Also one can synthesize the antibodies with the well charged natural aminoacid Ilysine, arginine or glutamic and aspartic acids) oligochains with the classical biotechnology help /for instance, 9, 10/.

Part VII. Chaperons: Principal Solution of Functioning of Protein Folding. Universal Recognistion Mechanisms.

In parts I-VI, the real interaction mechanism between the HIV envelope and CD4 molecules was evaluated but the serious proofs of the CD4-env interactions are vague and there is no sufficient data for such special pathways although there are, for Instance, some data with other viruses that also insistently indicate such “Du-2T” mechanism /for instance, 1, 2 and Refs.§3 here/ for “classical” env proteins, synthesized (as many other proteins) in ER-Golgi, where the env HIV proteins certainly and classically (“ER→Golgi”) have also the signal peptides /3/, which are well visible in extracts of contaminated cells, that are obviously the part of HIV macromolecule (because of anti-peptide anti-HIV-antibodies) /4, 5/. To prove clearly such important interactions Iwith Universal Antiviral (anti-Parasitic) preparations as consequence/, I had to resolve the most fundamental problem: of life sciences: .the process of foldlngs, functionings and recognitions of the protein macromolecules generally, where such gp120-CD4 receptor interactions is only a simple particular case.

The chaperons represent members of structurally unrelated protein famiIles that interact specifically with newly synthesized inascent) proteins and prepare them for their normal functioning. But how all these synthesized proteins with 'so different structures can be recognized by very limited quantity of structures (where, for instance, a quantity of antibodies active sites is enormous!)? Obviously, the Universal modifications of proteins must be responsable for such specific recognitions.

The glycochains are the best candidates. With help of very specific intercarbohydrate interactions, based on the important law of homologous intercarbohydrate interactions (Part II, §3), one must wait the natural solution. There are the clear -chaperon specialisations for each type of glycosylation: N-, O- and GAG- that justly simply determines their Universal specificities by their carbohydrate chains, like with IgG, where the specialized carbohydrate domains of the chaperon! protect. the corresponding proteins from aggregation due to their carbohydrate chains (Part VIII). The aggregation of proteins justly must take place due to the homologous interactions of their carbohydrate chains, like with lgG (Part VIII). Moreover such pure homologous aggregation (Interactions) of glycoproteins and proteoglycans, blocked by own free glycochains /6/ or stimulated by pure glycochains /7/ or by pure GAG chains /8/, already confirm the chaperon strategy. A pure aggregation of proteins, even after simple-non-enzyrnatic-galactosydation /9/ also confirms this. The homologous aggregation of glycophorins, introduced in liposomes after WGA lectin action /10/, also confirms such thesis although a situation is some more complicated (Part VII,§3). Larger concentrations of different samples (although some resembling) inhibited the homologous protein aggregation /11, 12/. This is justly the famous principe of the protein stablisation in sucrose solutions! So the specialized carbohydrate chains of chaperons protect the corresponding proteins from aggregation with homologous carbohydrate chains. Moreover, such development permits to resolve a very Important Universal property of the unknown protein functioning, which is hidden today under word “folding”.

§1. Endoplasnic Reticulum-Golgi Secretory Pathway.

There are two principal pathways of protein foldings: through endoplasrnic reticulum (ER)-Golgi and in cytoplasn. The ER folding begins from the growing. nascent polypeptide chain which is modified generally cotranslationally with N-linked sugars /13/. One is the main chaperon, of ER, calnexin (family) /14-16/, (exceptionally integral membrane protein) binds exclusively to monoglycosylated N-oligosaccharide chains only after removal of the first two glucoses /14, 17/. Virtually, all calnexin-linked proteins are N-glysocylated /14, 18/, although the calnexin (itself) does not have the N-chain /14, 19, 20/. Evidently, this chaperon is unic for the endoplasmic reticulum because there is no N-glycosylatlon in cytoplasm /13/.

Such monoglucose specificity could be well confirmed with the intercarbohydrate homologous interaction law. (Part II, §3). The monoglucose site is the very special motif: C—X—S—X—PC /19, 20/ and, very specially, there is the very similar invariant motif (C—X—S—X—P—G—C) in the calnexin, justly /21, 22/!

Moreover, the calreticulin (primary Ca binding protein) is also attached (at the same time) to the calnexin complex with the polypeptide with the monoglycosylated N-chain /17, 18/ although it has no such potential site /21/. The obvious complex between the calreticulin and calnexin 23/ (that can facilitate small contaminations) shows the possibility of such complexation. The similar structures between the calnexin and calreticulin /19/ can guarantee the homologous intercarbohydrate interactions between their O-chains, revealed due. to the potential O-sites /24/. Also the obvious complexes of the calreticulin (but not of calnexin) with BiP or grp94 (glucose responding protein) /23/, that must be created at later folding stages, and the presence of the different protein groups that interact with the calnexin and calreticulin /23/ confirm such thesis The last N-chain glucose trimming must be the calnexin dissociation signal /17/. The clear absence of the calnexin dissociation from a number of denaturated proteins (logically, glycochain attachment) and the dissociation of calreticulin, grp78/BiP, grp94, PDI, ERp72 (endoplasmic reticulum protein) with the ATP addition /25/ confirm the different character of the calreticulin dissociation (and attachment!).

The other important ER chaperon is the BiP/grp78 (Hsp 70 family), that binds soon after the chain translation into ER /15, 16, 26, 27/. It has many potential O-glycosylation sites justly in the substrate binding domain (and has no N- and GAG- /28/ potential glycosylation sites) /29/. So it could be specialized for the O-chains (with homologous intercarbohydrate interaction help). And lustly only the BiP accompanies the new synthesized proteins during their established recycling circuit ER→Golgi→back to ER /30/. And justly only during this path, they could be O-glycosylated in Golgi /31/. The accord of the mutual interactions with the chain associations of the BiP chaperon and IgG chains with their potential O-glycosylation sites confirms such thesis (Part VII,§3). Moreover, during the 1st Golgi “trip”, the NH₂ (COOH) proprotein proteolysis must take place because the furin and other proprotein convertases are concentrated ir Golgi /32-34/. During this “trip” in Golgi, the GAG-glycosylation (taking place in Golgi /35/) must happen also. In analogy with the specialized GAG chaparon (p50), that must attach specifically to the chaperon-“ship” of the HSP90 family (Part VII,§2), one.must suppose also the complex with the similar GAG binding proteins during the polypeptide circuit in Golgi. Already after the circuit in ER, the definitive chain creation must take place with the gp96/GRP94 chaperon (similar to Hsp90 in HSP90 family /15, 36/), serving (in analogy with Hspgo in cytoplasm /Part VII, §2/) as the framework for the large agglomeration with the other chaperons: mainly BiP, calreticulin, p50-like protein, Peptidyl-prolyl isomerase (PPI) and Protein disultide isomerase (PDI) /23, Part VII, §2/. There was the strong, yet astonishing, resemblance of the ER Hsp94 N-end and cytoplasmic Hsp90 with the heparinase active site without the heparinase activity /36-39/ but with the heparin binding /40/. And justly, the hsp94 (hsp90) must bind the p50-like protein with the GAG binding properties having the potential GAG sites /Part VII, §2, 28/. Logically, during such agglomeration creation, the hsp94 APTase activity takes place /Part VII, §2, 38, 41/. So the ER protein, specialized for the GAG chain protection, must exist.

Also, the proteins, having the potential GAG binding sites (for protection of GAG chains of synthesizedpolypeptide) exist in ER. The PDI has the heterogenous population with the conformational changes with ATP /24/, reflecting the PDI family presence /42/. And justly, the PDI has the strong invariant GAG potential sites (heparan sulfate) /43,28/, including the yeast PDI (Eugl protein) /44/. Also there is other mammalian PDI related protein /45/ with the GAG potential site (chondroitin sulfate like) /28/. At the same time, the other members of the PDI family do not have such sites, reflecting the functional complexity: folding and disulfide isomease (as such) activities /46/: Erp72 (431, P5 (phosphatase) /47/, including the yeast PDI (PPI-1) (48/!

But the PDI ERp61 family protein does not belong to the ER proteins. It does not have the ER carboxyl-terminal retention signal (but OEDL) /49/. “Erp61 did not rescue the PDI deficiency” in yeast as the other mammalian protein disulfide isomerase related proteins /49/. Moreover, the activity presence (attributed to ERp61) is very ambigous and variable according the results, obtained with the recombinant proteins /50/ /often with very different evolutionary cell origin that could be the very important error (Annex of Part II)/and even mesurement conditions of the PDI activity always do not correspond to those In vivo (with peptidyl-prolyl isomerase) and “obviously, we do not know” “what does PDI do in vivo” /51, Part VII,§3/. The ERp61 mRNA tissue distribution is very different from 2 other ER PDI family members /52/. Moreover, Invariantly, there is no GAG potential binding site as at PDI /50/. So this ERp61 (grp58) protein is clearly the active cytosolic phosphatidylinsitol—specific Phospholipase Ca /53, 54/ and it is only its resting (after N-end proteolysis) part that is active /53, 54/ (moreover there is only 1 contradicting work in vivo In situ). And justly (in tissues and cultured mammalian cells) there are, clearly, the two ERp61 forms /52, 55/, reflecting the presence of a (even) longer proproteins (than those determined by primary structure) /52/, that is the propriety of many signaling proteins (as casein kinase II, protein kinase C, other forms of Phospholipase C, NF-kB, P53) proteolysed by cathepsin L (major excretion protein) during signal (Annex AII). The (normally) present GR (methylated) peptide, serving for attachment to the “waiting” ribosome complex, must be normally at the undetermined yet 5′ part of 1st exon (usually until stop codon serving for stabilisation /Annex AII/). The clear (almost) absence of PI-PLC activity at ER (logically, yet ribosome attached chains can go inside even after synthesis /56/) or a contamination of, even transfected with Erp61, mammalian cells (less than 0.1% in cytosol of other type control cells) confirms location (on “PKC” transport vesicles /57/) of this PI-PLC-α. Moreover, unusually, in pioneering work /58/ with the PDI PLCQ activity (cloned PLC-α vector, given by Dr.Crooke /53/), the PI-PLC activity was indeed present before the Mono Q column and they found an obvious absence of activity in small peak (logically unproteolysed molecules—Annex AII) and did not localized (and evidently unidentified) the obvious real PI-PLC activity, known as the proteolysed fraction according the same Dr.Crooke work /53/.

For the case of glycosylphosphatidylinositol (GPI)-anchored proteins, which include the important case of famous priones (Part X), the required chaperons, are, evidently, present also in ER→Golgi /59/. The BiP is associated with precursor of GPI-anchored protein until modification by GPI /60/. It must mean, as in general case, that there must be the “trip” in Golgi (where there is no O-glycosylation and cleavage of N- and C- peptides) with BiP- with returning into ER. The GPI- transamidase is localized in ER /61/ and logically attaches the GPI after proteolysis in Golgi with help of the COOH peptide, logically making suitable conformation, for GPI anchoring.

Due to a presence of the ceramide contained in the GPI ) the GPI- attached protein clusters go with glycolipids (also ceramide based) /57/ in trans-Golgi and later to plasma membrane (PM) without dependence on glycosylatlon /62/. It is Interesting that the GPI with both types of lipid moiety contains the long 26:0 fatty acid /63, 64/. This supports the thesis, that the very long chain clusters at the outside layer of PM must modulate the thickness of PM at the zone of the main receptor, permitting for instance an association (with help of “valleys”) of peripheric molecules from the. cytoplasm /65/. So a presence of GPI anchored proteins must be essential for transmission of the signal.

In ER, the PPl participates in protein folding together.with PDI /36/. The β-subunit of PDI is situated near the α-subunit of the prolyl-hydrolase /51/, it means near the proline. Moreover, the PDI is identical to the glycosylation site binding protein /48/ and universally binds to the glycosylated universal peptides /51/, logically with O-glycochains (according to numerous potential sites /48/). The folding is finely regulated by PPI, having numerous genes /66/. There are always the two coupled prolyl trans-cis isomerisations /67 and Part VII, §3/ and a majority of unfolded species has the incorrect prolyl isomers /67/. The S—S bonds help to make the functional proteins and after reduction of the S—S bonds there .is an increased. synthesis of Hsp and Grp proteins (feedback) /68/. A precise function of these two enzymes remained obscure /69, 70/ because the mechanism of functioning of the protein folding was unknown (Part VII, §2, 3).

§2.Cytoplasmic Chaperon Machinery.

Generally, the cytoplasm folding is analogous to ER one although the cellular agglomeration happens on the ribosome (or ribonuclear particle- RNP): even O-glycosylation (GInNAc) takes place on the polypeptides, yet attached to ribosome (in cytoplasm) /71-73/. Consequently, the Hsp70 (family) attaches with TRiC (chaperonin family) to elongating polypeptide /15, 16, 74/. The Hsp70 must be specialized to the O-chains according to its numerous potential O-sites /29/ and the TRIC Justly has numerous special potential O-sites with Ile /75/, that must induct (although in Golgi synthesis) the special long core 2 chains (Part II). So the specialisations are also well traced.

The cytoplasmic glycosylations (often one GIcNAc residue) are some specific although the Rules are some similar to those in Golgi (Part II): nearby positive amino acids, proline and closeness of at least 2 Thr and/or Ser residues.Normally, refleetihg their homologous Intercarbohydrate interactions, these proteins with such glycosylation are mostly multimeric associations /76/.

The TRiC and Hsp7O are the ATPases /16/. Consequently, the principal complex with Hsp90, as an essential component, is formed /76/. “Assembly of protein complexes with Hsp90 is fundamental to biology of the eucaryotic cell” /76/. This complex, serving for folding (Part VII, §3) is analogous to definitive complex in ER (after circuit from Golgi) (Part VII, §1) and there is no, already, TriC (chaperonin) in this complex /76/.

The Hsp90 (“ship”) (having heparinase-like domain-Part VII, §1) makes. the complexes with p50 /76/, that is homologous to GAG binding protein /77/ and justly has the invariant GAG potential binding sites /77/. And the p50 (equal to important cdc37 /78/) binds to GAG even in vitro and there is an antibody, specific justly for protein site, responsable for GAG binding (near GAG potential site: DSG) /79/. Justly, the splicing p50 form without such potential site does not have such binding of the specific antibodies (it means GAG binding) /79/. The cdk4 molecule., which bind specifically the cdk37 (p50), justly, has.the strong potential GAG binding site /80/. The c-src and v-src molecules having the potential GAG site /81, 82/, justly attach to p50 molecules without hsp70 ones: O-chains) /83/.

Also there is the PPIs In complex with “ship” heat-shock protein hsp90 /76/. but without PDI (Part VII, §1) as well with protein phosphatase (PP5) /84/. Also the casein kinase II (CK-II) binds universally (by charge forces) to hsp90α /85/. The complexes with hsp90, hsp70, PP1, PP5 as well unliganded steroid receptors are found in cytoplasm as well in nucleus /76, 84, 86/. A presence of the functional phosphatase and CK-II in these complexes confirms a mechanism of a presence of these complexes in nucleus (Annex AI).

There is justly two types of complexes: hsp90-FKBP-52 (52 kDa FK-506 binding protein) and hsp90-CyP-40 (40 kDa Cyclosporin A Binding Protein—so called Cyclophilin-CyP) /76/. Logically, this justly, could reflect a localisation in nucleus of the 1st type of complexes /it is the “PKC” transport vesicle signal molecules having the (methylated) GR peptide at C- or N-end of proprotein/ (Annex AI and AII). After synthesis (but yet unfolded) at ribosomes in cytoplasm, each of these proteins goes to the constructing in nucleus preproribosome, naturally recognizing its “ship” complex and later they return into the cytoplasm with their proribosomes and wait only the activation signal (Annex AI,AII). For instance, all steroid receptors proprotein forms wait the signal in complex with hsp90 (and other attached to it molecules) that binds and neutralize the steroid binding domain /76, Annex AI, AII/. Justly, the phosphorylation by CK-II protects these molecules against proteolysis (Annex AI) and an activation of the phosphatase after action of steroid hormone. (Annex AI) facilitates the subsequent proteolysis by Cathepsin L (CL) that cleaves the proreceptor at C- or N-end /57/ with subsequent folding with liberated “Du-2T” peptides, that provoques enfin already intramolecular intercarbohydrate interactions of the similar oligosa-ccharides (it could be done also by receptor dimerisation) with receptor liberation from the “ship” with help of steroid /76/.

The inhibition of PP5, justly, leads to the permanent protection of complexes of steroid receptors with their presence In cytoplasm /87,84/. The limited proteolysis of PP5 (having also the GR sequence at C-end /88/) conducts to Its activation /89/.

As in ER (Part VII, §1), the PDI can be involved in the cleaved propeptide (Du-2T-like proteins) association. Justly, the complexes with FKBP are found in nucleus /76, 84, 86/ and there is only a undeterminated quantity of CyP-40 molecules that are in complex with steroid (p37 GR”-like /Annex AI,AII/) receptors /76, 86/. And the necessary (for the stocking complex activation) PP5 is justly the FK506 binding protein /84/. But the CYP molecules, which were found, justly, clearly only in cytoplasm /90/, logically, must characterize the complex that folds the “purely” cytoplasmic proteins without GR peptides and without “trips” in nucleus. The folding of these proteins is justly, different (VII, §3).

§3.Different Types of Protein Foldings—the Fundamental Bases of Their Functionings.

The above results help to resolve the very important, yet unknown, mechanism of the protein foldings (at least 3 types). The foldings take place with help of PPI where the 2 coupled prolines are. transformed from trans- to cis- state /68, 66/. Generally, the two S—S bonds are not absolutely required to maintain the protein in folded conformation /66, 91/. Logically, it is the two PPI enzymes, that make the trans-cis prolyl isomerisation of two not distanced prolyl residues /68/. Most unfolded species have justly incorrect prolyl isomers /68/. A difference between energies levels of cis- and trans- positions is small and the activation energy of each such transition is not large /68/, but, logically, after both simultaneous transitions, . the return is normally impossible for one transition during all subsequent protein functions where for instance, the energy after substrate addition must be temporary stocked as tensional one during general enzyme action, due to the dynamic multiglobular structure of each stbtnit /92/. permitting the rigid tensional relative displacement of such globules (creation of potential stocked energy due to liberation of energy in enzyme-substrate interaction, for short time of the catalysis- Yu.I.Khurgin- personal communication). Such natural unfolding transitions are absolutely unknown! Justly, this so called folded state of protein is made by distorsion mechanism /69/ (here the PPI acts together with PDI). Even after denaturation in urea, such “defolding” transition is impossible, except after cleavage of N-end /93/.

In the case of ER-Golgl way of the folding /as in the case of IgG (immunoglobulin. G) (Part VI) or MHC class I molecules (Part IX)/ (Part VII, §1), there is a folding of the protein with help of the cleaved propeptide attached to the macromolecule (distorsion mechanism with help of PPI with PDI /69/). The membrane viral glycoprotein folding appears to be complete upon cleavage (in Golgi /94/) of the p7 peptide from E2 /95,96/. With badly changed polypeptide there is no enzymatic activity of the carboxypeptidase (ER→Golgi) /97/ as consequence of uncomplished folding in ER. It is clear that (as in the case of folded functioning IgG /Part VIII/) during the obligatory attachment of this peptide (“Du-2T”), the carbohydrate chains will have the interactions intramacromolecular, instead of intermacromolecular with oligocarbohydrates of the chaperons (liberating from them). With dissociation of such peptide later, after folding (already done irreversibly.in ER). there is the strong conformational change with new dissociation of intercarbohydrate homologous bonds (intramacromolecular) as in the case of IgG or MHC-I, where the peptide (logically) could be even taken from other protein (visibly “Du-2T” is similar) (C-end of lipoprotein- associated coaglutination inhibitor) /98/. Such liberated carbohydrate chains serve for new attachments to similar oligochains (ADRESS, LAW OF RECOGNITION IN CELL) at PM or ECM (extracellular martix) or to complement or to the TCR (Part VIII,IX). The apolipoprotein A-I without cystein residues is maturated in ER with (logical) cleavage of peptide in Golgi /99/. In the case of the folding in cytoplasm there are the two major folding cases: signaling “PKC” vesicle transporting molecules having the GR peptides and making the “trip” in nucleus /Annex AI, 57/ and “pure” cytoplasmic molecules. The classical example of molecules with “GR” peptides is steroid receptors (Annex AI,AII). Their folding must take place on the ribosomes in cytoplasm with help of hsp90 “ship” machinery (Part VII, §2). The neutralization of hsp90 action during folding leads to loss of the steroid binding activity at all /100/, it means there was no folding with PPI activity. The new assembling of the chaperons with receptors in nucleus in constructing preproribosomes supports the hormone receptor activation state in cytoplasm where the constructed specialized proribosome with proteins (RNP) goes in cytoplasm and waits the activating signal /Annex AI,AII, 57/. In the case of the “PKC” vesicle transport “GR” protein lipocortin 1 (inhibitor of phospholipase A2), a modification of N-end peptide during folding also leads to loss of action of the lipocortin 1 /102/ (no folding!) where such proteolysed n-peptide really exists in vivo /103/ and it serve (in these cases) for attachment of synthesized proteins in preproribosomes-preribosomes-ribosomes and general correct structure at folding.

The case of folding of “pure” cytoplasmic proteins, representing many enzymes, is quite interesting. The cytoplasmic synthesized enzyme rhodanese (developed case) that goes to mitochondrium, has a number of potential O-sites and a number of prolines /104/. The N-segment in chain is necessary for folding (evidently with PPI) and this folding is necessary for enzymatic activity /105/. The proteolysed N-segment associates with whole molecules, although the whole molecules (folded already!) have an activity independently on presence of cleaved peptide on it /93/. But the N-end attached peptide (“Du-2T”) (as in the case of ER→Golgi secreted proteins) must be hidden with help of homologous carbohydrate chains and it is important for enzyme targeting and translocation /93/. During RECOGNITION of the enzyme carbohydrate chains of the homologous chains of the enzyme targets, there is a. dissociation of this peptide, that (in positive feedback) reinforces the intermacromolecular (already) intercarbohydrate homologous interactions. In the case of other important cytoplasmic protein src-kinase there is no tyrosine kinase activity (it means without hsp90 chaperon presence at beginning) although the hsp90 has no influence for activity for already folded exogenous src kinase /106/. At PM, logically, the GAG chains could be liberated to interact, for instance, with the homologous GAG chains (adress!) of the main receptor /57/. Justly, the quite different behavior of the two types of PPI in cytoplasm: cyclosporin A sensitive and FK506 sensitive (Part VII, §2) confirms well such 2 types of the cytoplasm foldings and the general mechanism of action of the enzymes.

Part VIII. IgG Priones as Clear Example of the Most Studied Case.

The immunoglobulin G (IgG) (even as antibody) behave as classical prione aggregates (that must mean that a number of IgG. provokes other ones in common aggregates), in loosing real Little Protein Du-2T (mol.w. ˜1500 /1, 2/). At a loss of such Du-2T protein, the IgG conformation, justly, become more rigid /2/. This Du-2T protein, logically, is originated from the proprotein sequence, beginning from stop codon /Annex AII, 3/. The interaction with antigen at active site (N-distal part of macromolecule) also leads to a dissociation of Du-2T in CH2 domain /4, 5/ due to brake of the interactions between two homologous carbohydrate chains, that hide this Du-2T in a cavity. Such signal (as also analogous thermal IgG aggregation /6/) must make the complement activation /5/. Such conformational switch (at distance) passes through the IgG folded domains V and C1. Such possibility is a consequence of the general folding of proteins in ER→Golgi. One can conclude that at assembling of whole IgG molecule there is the IgG folding /7-9/. The chaperon BiP binds each heavy chain with their consequent aggregation at Fc part but not at Fab part. At the same time, the BiP binds to each light chain. The interchain V domain S—S bonds are formed before BiP dissociation /10,11/. The next heavy-light chain interaction with dissociation of BiP and mediation of dimer formation with help of only Cl domains takes place visibly with help of long core 2 O-chains which takes place during BiP dissociation with intraC1 domains S—S bond formation.

A clear presence of isoleucine /which characterize longer chain core 2 glycosylation (Part II)/, justly only in the C1 regions of heavy and light chains /13/ confirms. their mutual interaction with help of homologous intercarbohydrate interactions. Naturally, these longer carbohydrate chains prevented the earIler S—S intra-C1 domain bond formation. The Fab (IgG) prolyl isomerisation that determines the foldings takes place after formation of the quaternary structure (Part VII) with action of the disulfide isomerase /13-15/ on the interchain S—S bonds /16/.

So such classical formation of folded V and C1 domains with closed S—S bonds /17/ permits the signal (transmitted through long distance) switching from the N-part active site through above domains to CH2 domain.

One can see also the switch of the direct complement activation with mannose binding protein (MBP) with help of IgG /18,19/. This can be happen when there is no terminal galactose residues and the GicNAc is accessible directly. The MBP is structurally related to the C1q subcomponent of the C1 unit of complement and its complement activation is similar /18,19/ with specificity to N—GIcNAc /20, 18/ in special carbohydrate binding domain where, justly, one can wait the core-2 chain with GINAc /20, Part II/. Evidently, in this case there is a direct brake of the IgG interchain (intramacromolecular) homologous interactions and a dissociation of Du-2T protein /2, 4/ with complement activation /5/.

There is the other example of the direct IgG interactions. They happen between the IgG CH1 region and the complement C3a subunit (alternative complement activation) /21/ where the both established interaction sites have, justly, the strong potential glycosylation sites. Justly in the case of the class IgE (without complement activation), there is no potential O-glycosylation sites at this site /22/.

Part IX. Mechanism of Switching of the Cytolysis with Help of MHC Class I-Peptide Interaction with TCR and General Chaperon Make Up.

One can certainly see an existence of such powerful peptide. in the case of MHC (major compatibility complex) class I molecules. Such above development permits me to have the thread of Ariane with help of which I can resolve such important problem as the lysis of contaminated cells with help of the MHC class I molecules. Evidently, the MHC class I molecules are not the total exception for chaperons. As the ER→Golgi transported proteins, the MHC class I heavy and. light chains are contranslationally translocated into ER and are classically glycosylated and make the native heterodimer. with help of different chaperons (Part VII) as calnexin and BiP /1/.

A presence of the peptide induced conformational changes of the MHC class I molecules is observed and it clearly means that due to its dissociation, a strong conformational change of the MHC molecule must take place. Such loosely packed not very specific peptide, logically, must be folded under pressure of the α-helices of α1 and α2 subdomains (their COOH interaction parts)/4/. The binding is situated between the helice α1 and α2 domains and a floor is composed by the β-sheets (as grounds) from both domains /2, 4/. Although the α1 domain has no S—S intrachain bond, there are the several prolines in this domain /5-7/. So at the peptide pulling by TCR, the switching movement of these α-helices must take place /4/. The TCR buriesalmost all available peptide surface /8/. Moreover, justly, a very exceptional presence of the charged amino acids within predicted transmembrane domains at all components of TCR /9/ makes the vertical position of the receptor chains unstable and must permit such pulling movement of TCR with peptide.

A changed /after such peptide pulling—“dissociation”/ MHC conformation, logically, must permit to the unpaired. N-chain (in invariant site /Asn 86/ between extremely variable domains α1 and α2 /10/) to interact with the, also unpaired and invariably present/11/, N-chain of the TCR Va domain. This interaction must be specific and it could help to permit to transmit the signal from the active site of TCR through 2 Ig-like domains with closed (by S—S bond) folded loops and two nearby prolines (Part VII) to the S—S interchain bridge (like in igG molecule /Part VIII, 12/). Exceptionally, the length of these S—S linked. Ig-like chains is different /13, 14/. Moreover, the Cα domain is more disordered and its folding is unsymmetrical to the Cβ domain /4/ that clearly must facilitate the Du-2T-like peptide dissociation (protected logically by symmetrical N-chains nearby the S—S bond /13, 14/) (Part VII,VIII), leading to strong conformational changes of TCR α,β chains /4/. The TCR αα-homodimer cannot be active with MHC /15, 16/. Such conformational changes of TCR must permit the more intensive intercarbohydrate interactions between O-chains of the α1 and α2 domains of MHC and TCR in increasing general affinity. Justly for interacting CDR1 (complementarity-determining region) and CDR2 areas of MHC (major histocompatibility complex) /4-6/ and CDR1, CDR2 and CDR3 areas of TCR /11, 17, 18/, there are the variable potential O-glycotylation sites permitting specific interactions.

From ˜370 amino acids there are only several invariant ones (but well distant in space) /10/. So it is obvious that the specific allotype determinant (at least several close Invariant amino acids) cannot exist for such great TCR active site family. Naturally, it is the specific carbohydrate chains (pattern of which, justly, is specific for the species and even individuals, organs and tissues /19/) that must characterize the MHC and TCR molecule allotype where their partial coincidence must determine their affine homologous intercarbohydrate interactions. Also the N-linked oligochains possess the allotype specificity /19a/ and there are many potential Clinked chains at the different MHC-I and MHC-II proteins /19b/. Moreover, the MHC glycochains and monosaccharide pattern Inhibit specifically the allospecific cytotoxic cells /19c, 19d/. Such carbohydrate allotype pattern determines the Inherent TCR repertoire presence (specific for MHC) /20/. The cytotoxic T lymphocyte polyclonal answer activation (without MHC presentation) directly by the attached (to cell) carbohydrates, and a stronger answer for the carbohydrate part of the peptide /21-23/ can be explained by the rigid carbohydrate determinants and the evolutive presence of a large number of the TCR and MHC active sites against such concrete important determined structures. So, even solely, such carbohydrate origin of specificities of the alleles, proves the Law of Homologous Intercarbohydrate Interactions.

So such increased conformational change must induce the CD8 molecule (CDR regions of Ig-like domain) to react with the α3 domain of the MHC class I molecule (residues 223-229) /24, 25/. Also there is a mutual presence of carbohydrates (O-chains here) in contact areas /25, 26, 5, 6/. Due to a presence of the prolines and numerous O-chains, nearby the interchain area /26, 27/, one can wait a presence of the Du-2T-like proteins that dissociate after the conformational change in Ig-like CD8 domain (with interchain S—S bond) (Part VII,VIII). Logically, the peptide for α3 MHC I domain must be also made from the propeptide with help of the proprotein convertases,responsable for the endoproteolytic processing of the proproteins, like furin, concentrated in trans-Golgi network (Part VII). But in the α3 region of MHC, the dissociation of its “Du-2T” must be due to direct dissociation of the covering Interacting carbohydrate chains (Part VII,VIII). There is no prolines in α2 domain for Ig-like signal transduction from “active α1α2 site” /5, 6/. The β-microglobulin, having many atomic contacts with underside of the floor and with concerved α3 dornaln /2/ and having many concerved carbohydrates /28/, dissociates from heavy chain after peptide “dissociation” /2/.

There are many MHC class I molecules synthesized in the same cell /4-6/, which cannot represent the numerous types. So the MHC class 1-βheterodimers wait the corresponding peptide in ER /29, 30/ from gp96 or prolyl isomerase or calreticulin (Part VII) to have a possibility to leave the ER and Golgi for plasma membrane. The extreme exceptional sensitivity of the MHC-peptide “ligand” in eliciting the cytolytic T-cell response /31/ confirms such thesis. In order to have sufficiently large number of receptors in the cultured TCR complex, the triggered aggregation of unliganded (!) TCR receptors (˜180 par one ligand!).-nust.take place /32/, that clearly must be similar to the prione actions (Part X). The numerous dissociations and associations of the affine. TCR-MHC-peptide complexes are far from reality. For instance, the strong conformational changes .of the MHC chains already take place after “1st” (and last!) interaction with TCR /2, 4/ and already this new state triggers the creation of the complexes of TCR-αβ with CD8, CD3, CD45 (CD4) /33-35/. Moreover, such new receptors' re-interactions (with their affine targets) have no precedents (at mitosis also) and the permanent presence of the liganded receptors during at least ˜8 hours is obligatory for mitosis /Refs.36/.

Such large aggregation takes place with very well glycosylated molecules as CD8 with. additional multimers or leukosialins /37/. Moreover, the special unsymmetric chain dimerisations (CD8, TCR, MHC) /27, 4/ facilitate such strongly concentrated aggregations which lead to a melting of PM due to the local dehydrations (after homologous intercarbohydrate “chair” interactions /19/) and (at the end) the target cell lysis.

Part X.Net Solution of the Process of Formation of Priones: Primary Cause of Mad Cow and Creutzfeldt Diseases is the Artificial Dissociation of “Du-2T” Peptides.

The case of prione proteins (PrP) is only the particular case of the secreted through ER→Golgi /glycosylphosphatidylinositol (GPI)-anchored/proteins /1-4/. The PrP certainly has general characteristics of such folded proteins: the 2 nearby prolines, the S—S bond, important for general conformation, the 2 nearby N-glycosylation sites /1, 3, 5/ and they are made and folded with help of the corresponding chaperons /Part VII, 6/.

It must be normal functional protein with the GPI anchor that modulates a creation of the intercarbohydrate network near main receptor /Part VII, 7/, re-entering through subcellular compartment with cholesterol-rich membranes /8, 9/, evidently with dissociation of Du-2T- like peptide and opening the carbohydrate O- and N- chains for homologous Intercarbohydrate interactions (natural transition PrP^{C} /cellular/→→PrP^{“Sc”} /scrapie/). Naturally, the highly conserved N-glycosylation sites are essential for PrP^{C} function /10/. Consequently, its expression is enhanced at lymphocyte activation /11, 12/ and the charged molecules stop a propagation of the signal /7/ in stopping the “scrapie” accumulation /13, 14/. Evidently, the cross-linking takes place at receptor-glycosphingolipids complexes /7, 9/ in calveolae-like membranous domain, rich in gangilosides and ras proteins (“railway station” for “PKC” transport vesicles) /15, 16, 9/. Naturally, uncomplexing PrP^{C} is soluble in various detergents but the cross-linked PrC^{Sc} forms the soluble aggregates /17/. Levels of the PrP mRNA are developmentally regulated /17/. Naturally, there is the decrease of the signal with the decrease of concentrations of cellular isoform at scrapie /18, 19/ and the synthetic prione protein fragments (without corresponding carbohydrates) are toxic but those from natural sources (with corresponding cross-linking carbohydrate chains) increase the signaling /20/.

Naturally, as with IgG, the folded form with small Du-2T- like protein (PrP^{C}) is easier proteolysed /2, 21, 8, 17/ and hasthesame amino acid sequence /2, 8/ and, even, does not differ. at level of the posttranslational chemical modification /22/. Naturally, as in IgG (§VIII),the “Du-2T”-must be hidden bythe nearby interacting homologous N-chains and consequently there is the species specificity (the same sequences) of the cell free prione conversion /8, 23/. Evidently, the point mutations (natural and exprerimental) destabilize the tertiary structure of the PrP^{C} and lower a barrier of transition to the PrP^{Sc} /1/. The aggregated scrapie form destabilizes easier the cellular prione form /1, 2/ in reacting with their carbohydrate chains and in provoking a dissociation of the Du-2T-like protein (as in the case of IgG-Part VIII) where the PrP^{Sc} concentrations did correlated with Infectivity titers in scrapie-infected mice /24/.

The region of “Du-2T” protein in exon 2 (cleavage of C-region is not known here) is the area of the highest homology /25/ and the fused N-end of yeast prione does not produce the N-peptide, necessary for folding (Annex AII, Part VII): no folding-no prione-like properties /26/. Moreover, with transgene operation, the incubation scrapie time is often shorter /8, 27, 28/ that could be connected with perturbation of the important propeptide sequence.

Evidently, many other folded proteins could be (at some destabilizations) transformed pathologically into aggregated forms with dissociated Du-2T- like peptides, where the carbohydrate chains are ready already to act and to provoke nonnormal aggregations. A presence of the even cytoplasmic form of the priones (translation termination release factor-eRF) /29/ as consequence of the foldings of the cytoplasm signaling proteins (Part VII) or the real functioning (during the signal /in situ/) of numerous TCR molecules as priones (Part IX) confirm a general character of such prion phenomenon.

The definitive proof of my conclusions is elementary. The dissociating (at conversion PrP^{C}→PrP^{Sc}) Du-2T-like peptide, a part of the prione N-end must exist in this particular normal case t6o.

Annes.

AI. Universal Signal Transduction from Plasma Membrane to Nucleus: pH Increase→Increased. Cathepsin L (CL) Synthesis→Liberation of Stocked on Ribosomes CL→Nuclear Factor Release→Gene Activation . . .

Introduction.

In reality (unknown yet), the main characteristic of the Classical Cellular Signal is to establish the Vectorial Universal Transport of the substances (including Ca²⁺ ions) between the plasma membrane (PM) and the special regions of the Endoplasmic Reticulum (ER) (“calcisomes”) to respond for cellular organism needs /1/. Such known vectorial transport during the cell movement or the axon cone growth is only particular case of my general conception.

The indiscutable reality of a presence of such transport, one can follow from too simple (In execution as well in very veritable relativity of data) works of A. L. Hodgkin and coworkers /Refs.1 ,Refs.2/ made already a half century ago. “A small patch of radioactive ²⁸Mg (and ⁴²K) injected into axon broadened considerably. A similar patch of ⁴⁵Ca showed hardly any broadering” /3/.it must mean that the omnipresent networks exist in cytoplasm where the size border between almost free and almost restricted diffusion is situated principally between the size of Ca²⁺ and K⁺ ions and consequently a smaller H⁺ and Na⁺ ions must have almost free diffusion. So numerous experimental works with rapid Ca spread from PM to the nucleus /4-6 with Refs/ rmust be interpreted only as the directed vectorial transport /1/. The “markedly asymmatric profile” of such Ca spreads, the outward current (circular!) and the insistent periodic discrete Ca peaks (in space and in time) /4-6 with Refs/, justly corresponding well to my predicted “Protein Kinase C” (“PKC”) transport vesicle Cycle with a number of intermediate arrets due to the descrete belts of the propulsing circumferential “acto-myosine” machinery /1/, serve as the confirmation.

But how to build these “railway stations” (at PM and near the nucleus) and “railways” of the transporting “PKC” vesicles from beginning when there is no yet the transport to transport the material for these communication means? In such case, the cell utilizes the stocked proteins and the first activation of the nuclear genes (immediate-early genes) takes place very quickly without this transport.

§1.Na⁺/H⁺ Exchanger Activation with Rapid Cytosolic Diffusional (All Over) pH Increase. Increased Protein Synthesis.

The Na⁺/H⁺ exchanger (different isoforms) presents in all eucaryotic cells where it makes the pH elevation after many different signals /7, 8/. This pH elevation can be ;switched after activation of the special forms of the PKC /8-10/. Such elevated pH is present during hours (even after simple phorbol application) /9/. Only stable increase of the exterior pH is sufficient to provoke a more Intensive cell growth with 2-4 times higher density /11/ and logically, the simple diffusion of the small H⁺ (and small Na⁺) ions through the cytoplasm must reflect such changes (FIG. 4). But the increased interior. pH stimulated a more intensive protein synthesis as was shown for many cell types /11,Refs 12-14/. Logically, in this case, the consecutive synthesis of all proteins in cytoplasm increases and, normally, such addition (to protein synthesis) had no to make strong changes. But in the case of the proteolytic enzyme Cathepsin L (CL) (papain-like cystein protease), a situation is quite different. Having the long-living /15/ and very stable long-size form (at normal pH) /16, 17/, these proteins could “wait” such critical moment during a long intercycle pause to switch very important events.

§2.N-methylation of Proteins and Protein-Nucleic Acid Interaction.

To understand such events, we must understand a nature of the protein- nucleic acid interactions. For example, the proteins participating in the mRNA biogenesis (hnRNP) have the special motif structures (β-sheets and α-helices) for RNA binding /18, 19/. The ribosomal proteins have the special arginine rich (methylated) motives (ARM) (particularly, IN THE MIDDLE of their structures) for binding the rRNA hairpins /18,20/, permitting the specific interactions with rRNA.

But this rRNA serves also for attachment of a number of other proteins in the preproribosomes (pre-ribosomal particles- rRNA and ribosomal proteins) in nucleolus. For instance, it is clearly shown that the basic fibroblast growth factor (bFGF) has several forms encoding the same protein with different lengths from the N-end /21, 22/. And only the higher molecular weight forms, being dimethylated at arginine, are situated at the nucleus: preproribosomes (Part VII) and at the proribosomes and ribosomes (Part VII) in cytoplasm whereas the short (18 kDa) form is found only in cytoplasm /21, 23/. Moreover, the limited proteolysis of the higher form making its transformation into the short is clear because after a transfection of only longer forms, One can find in the medium also the short form /21/. There is a number of other N-methylated (asymmetric dimethylarginine-ADMA) proteins: major nuclear protein-nucleolin/24/, nuclear protein-fibrillarin /25/, ribosomal protein S2 /26/, heat shock proteins /27/, actin /28, 29/, visibly tubulin /29/, a number of the undetermined yet proteins that are bound with ribosomes /30, 31/. It is shown in many cases that this methylation takes place at N-end or C-end of the molecule /21, 22, 24-26/, that justly confirms a possibility to detach the molecule from its anchor with help of the limited proteolysis. A presence of several forms of the bFGF /21/ and nucleolin /24/ confirms such proteolysis. So these ADMA protein ends help to attach different proteins to the rRNA in the preproribosomes and later in the proribosomes and ribosomes with liberation during the limited proteolysis switched with the signal.

The extracoincident facts confirm such model. During the dystrophy (with excessive phospholipase A2 PM lysis) there is a strong process of the cell reparation, it means an intensive transport of substances /1/ and justly with the dystrophy, there is a significant increase of the dimethylarginine in urine /32/, confirmant such proteolysis during the cell transport activity. Moreover, lustly this ADMA is the specific inhibitor of the nitric oxide synthase /33/, synthesizing NO, that inhibits the mitochondrial respiration /34/. But justly this mitochondrial respiration is absolutely necessary for the “PKC” transporting /1/ during cell signal activation. It means the NO additionally blocks the mitochondria and consequently the transporting does not take place during absence of the signal.

Moreover, the 2nd limited proteolysis (proribosomes-ribosomes /Part VII/), with detachments of the ADMA rich ends of the nucleolin and fibrillarin, must take place in cytoplasm. The fact that the N-end of the nucleolin (without ADMA) is proteolysed in nucleolus (until 50 kDa COOH part) during the proribosome formation (Part VII) /35, 36/, a presence of the (proteolytic) fragments of the nucleolin on the cell surface /37/ (after, logically, proteolysis), where the 30 kDa fragment, logically, must resemble a resting part after 2nd proteolysis in cytoplasm at the ADMA C-end, confirm such conclusion. The CL accounts for the main part of the cystein protease activity in the cell /38/, being the major excretion protein (MEP) that is different from any protease /39, Refs.40/.

A presence of complexes of the attached proteins with ribosomes, waiting the signal, is proved by a number of other data. The estrogen receptors (that are detached by limited proteolysis after signal /41/) are found complexed with ribosomes and after signal, their anhors. are cut and they dissapear from cytoplasm into nucleus /Refs.42/. A localization of the actin proteins and their mRNA coincides and a localisation of the actin mRNA parallels an elaboration of the cytoskeleton during cell growth /43, 44/. The mRNA and rRNA are localized together /45/, justly, at the zones of the signal action (cone growth and cell motility are only particular cases of the general complete signal /1/). A presence of the rRNA in developing axons and dendrites /46, 47/) (where the ribosomal transport with help of the axon's subcortical circumferential regions is well visible /49/), a presence of the β-actin mRNA in the growing axons /49/ and in the lamellipodium /47, 50, 51/, a presence of the tubulin mRNA in the growing axon cone /49/, a presence of the mriNA of GAP-43 and MAP-2 as well of mRNA of the inositol triphosphate receptor (type 1) in the growth cones /46, 47, 52/ and a presence of the β-actin mRNA near the PM after signal in fibroblasts /53/ confirm such data.

The selective targeting of these complexes is directed by 3′-end of the mRNA /54-5/. One can see that these special proteins, synthesized and cleaved on specially moved ribosomes, justly belong to the proteins of the “PKC” vesicle transporting cycle /1/. A very “strange” fact that the mRNA of the transmitter receptors (justly being close to these ribosomes) is not present at such places, in spite of the very intensive search /46/, confirms such conclusion.

Although the arginine N-methyltransferases are very predominant /57/, there are evidently other highly specific protein methyltransferases /Refs.58/. But their regulation with biological purpose must be different. For instance, the carboxyl dimethylation of the protein phosphatase 2A takes place with other specific methyltransferases /59/.

§3.Autoactivation of Cathepsin L.

The switching protease is the Cathepsin L or MEP. This very special molecule also has several forms with different lengths /60/. Moreover, the best substrate (at pH=5.5) “with exceptionally high (contant) rate”, that have been found for this enzyme, contained the methylated arginine: Pro—Phe—Arg—CH₂Cl /61/ and the CL cleaves often proteins in the arginine region /62-FIG. 3), having a strong capacity to proteolyse the proteins in difference with Cathepsins B and H /62/. Its secretion increases after alkalization (as the bFGF secretion /63/) of the intracellular vesicles /63, 64/, that means that there is no participation of the Golgi (with acidification) but it must be the “PKC” transport vesicle signal exocytosis (justly unfindable pinocytosis) /1/, showing an increasing presence of the CL in cytoplasm. Moreover, the ionophore monensin (increasing pH) decreases a quantity of the activated form CL in cytoplasm but increases an accumulation of the largest form /64/: the autolysis of CL must be weaker at higher pH /16,65/ but the synthesis of the longest form on the ribosomes in cytoplasm is more important (Annex AI, §1).

Justly before a beginning of the signal there is an accumulation of a number of methylated proteins (stable long CL form included) on the rRNA of the ribosomes in cytoplasm /1, 41/. Evidently, there is a presence of the “zero level” of the cell activity and protein synthesis. At an appearance of the new synthesized free CL molecules at more alkaline pH, the autoprocessing of these molecules at such, yet neutral, pH must take place (FIG. 4). Logically, these CL molecules do not have the signal peptide /Refs.66/. Once the mature active CL is generated, the proCL is rapidly activated /67/. This is the essential point of the signal activation. Only an increase of pH of the exterior medium is sufficient for the CL secretion /68/ from the cytoplasm, evidently due to increased at higher pH synthesis. Subsequently, these active autoactivated short form of CL must liberate the accumulated (in the Inter-signal time) CL molecules and other special proteins that are distinguished by ADMA at their chain ends and it also activates the ribosomes in cytoplasm by limited proteolysis of particular proteins taking part in the ribosomal work /41/. The CL increased synthesis by ribosomes and. the ribosomal activation with CL, evidently, must act in chain of positive feedback.

The fact that the CL has (justly at proteolysed N-peptide) the sequence GlyArg /Refs.66/, that must servir for the rRNA attachment, confirms well these conclusions because “the presence of glycine on the N-flanking side of the methylable arginine was shown to be an absolute requirement for methylation /58/.

§4.Activation of the Alkaline Phosphatase (AP).

There is also the other factor that helps to make the limited proteolysis of proteins: elimination of their protective phosphates by the AP. At an increase of the pH, an activity of the AP increases beginning justly at pH-7.0 /691. And for instance, with an action of the EGF (and TGF-β) there is a rapid shirt increase of the AP activity /70/. At an action of the steroid hormones, this activity increases in ˜100 times /71 with Refs./. An increase of an activity of this cytoplasmic enzyme (marker of differentiation /71/) can be seen also at the bacterial infections /72/ and with cancer /72a/. (In all cancerous cells, an intensification of this cycle signal transport “PKC” vesicles must take place and justly, all protooncogenes participate in such process /1/). Such unknown yet function of AP is indeed the dephosphorylation of the protective phosphorylation (in the area of the limited proteolysis), made by vesicular casein kinase II/1,41/. In very good convergence, this AP makes its preferable action justly near the arginines (with.glycines) /72b/.

§5.Limited Proteolysis of Nuclear Factors and Nuclear Signal.

The early appearing nuclear factors (as NF-kB, c-jun, c-myc) conduct the signal due to their liberation in the cytoplasm by the limited CL proteolysis after a pH increase. One can clearly confirm a presence of the p15(p50)/p65(RelA)/IkB-α complex with ribosomes in the. cytoplasm before the signal with help of an information about their primary structures.

If to continue a reading of the “open reading frame” (ORF) upstream of the “initial codon” (often not well determined /41/), one will discover very systematic particularities: always in p105(p50), IkB-α and p65 (RelA) there is a presence of the stop codon at some relatively short distances /Refs.73; 74/. For instance, for different p105, the stop codon is found already at 19 or 12 or 19 amino acids upstream of the “initiation codon”. Obviously, as always for many other special molecules serving for “PKC” transport vesicle machinery /1/, such dipeptide had to be sufficient for asymmetric arginine dimethylation /41/. One can conclude here the precise mechanism of the NK-kB activation. The IkB-α, complexed with p105(p50) and RelA molecules, “wait” the signal in cytoplasm /75/ resembling in part the scheme from /76/. All these molecules are attached by such special ADMA also to the rRlNA of the cytoplasmic ribosomes. There is the rapid NF-kB activation by the proteolysis (evidently, without protein synthesis /77/), afterwards it goes to the nucleus in activating the special genes /78/ that are absolutely necessary for melting of the PM surface (“railway station” for “PKC” transport vesicles, including necessary integrin “sub-station”) /1/. Justly, with the pH increase, the AP antiprotective dephosphorylation of IkB with subsequent limited proteolysis /79/ by autoactivated CL (including a detachment of all stocked proteins from ribosomes) must take place. An appearance of the IkB with higher molecular weight (due to such peptide), with an inhibition of the phosphatases and proteases /80/, confirms this conclusion. An inhibition of the cystein proteases (including CL) blocks a degradation of IkB-α/81/ where not only the chymbtrypsin-like cell protease takes part in activation according to /80/. After such limited proteolysis there must be the complete proteolysis with proteosomes /82/ to eliminate definitively the IkB-α protection of p105 protein. The IkB-α is degraded while the cytoplasmic complex, bound to Rel ,and the phosphorylation are not sufficient (at least) for dissociation /83, 84/. Justly a degradation of the IkB increases the p105 processing /73, 85,Refs.86/. The subsequent limited endoproteolysis of the p105 (with CL) is necessary for subsequent complete degradation of the detached C-terminal portion already with proteosonmes /87/. The limited proteolysis In making the p55 chain from p100 (It means approximately at exon 15, where the CL action could be possible due to GR peptide /89/) confirms such action and explain the strange exceptional “pity” of the proteosomes for N-part of p105 (p100) /82, 87/. Evidently, a detachment from rRNA also takes place with CL proteolysis and the protease inhibitors with detaching detergent provokes an appearance of the forms of NF-kB with higher molecular weight /80/.

§6.The CL Activates the Ribosomes in Cytoplasm Ribosomal Tournover.

The above data help to clarify the complex pathway of the ribosomes. After an initial activation of the preproribosomes In nucleus, the CL must proteolyse (in proribosomes in cytoplasm) their particular proteins at the GR peptides (that bind their corresponding sites at the preribosomes, migrated from nucleus) in cytoplasm. These nuclear proteins are. nucleolin and fibrillarin and several ribosomal proteins like L5/Refs.90/ and several other preribosomal proteins /1, 41/. The nuclear proteinsas the nucleolin and fibrillarin, logically, served only for ribosomal partial assembly /91,92/ and later as the “fusible” and their function had to be principally accomplished after CL proteolysis that is confirmed by a difficult detection of the nucleolin pool in cytoplasm /93/. Justly, these proteolyses must activate the proribosomes and justly, because of this ,the eucaryotic (pro)ribosmes can be activated In Vitro only with reticulocyte Yate that must, logically, contain the activating CL.

During already G1 signal (or signal at G0), a part of proteins, synthesized on such ribosomes .(with GR ends in situ) (evidently unfolded yet-Part VII), goes to nucleus and attach to the rRNA in nucleolus /41/ with help of the nuclear proteins as. nucleolin, B23, fibrillarin /91, 92/. The means for penetration into the nucleus are described elsewhere/94, Y.Z. Application FR-98-06910, retired). A transcription of the ribosomal genes depends on a presence of these nucleolar proteins /95/. The “NH₂” 1stproteolysis of nucleolin (“NH₂”-non-GR” part is attached to the chromatin /96/) is necessary for the pre-rRNA transcription /97/. A finding of only minor amounts of putative degradation products (small proteolysed NH₂ part) /96/ confirrns such proteolysis. Logically, it must be the serine or cysteine nuclear proteases, activating with their signal /98 with Refs/ because the inhibitor leupeptine, inhibiting the proteolysis of the NH₂ part of the nucleolin, inhibits the synthesis of the pre-rRNA /96/. After already a beginning of the signal (or after mitosis) (also at the end of the signal with stocking) the proribosomes go from the nucleus into cytoplasm with help of the nucleolin and fibrillarin /93/ and of the mRNA serving as a guide (by its 3′ part) for the cytoskeletal localisation (near “interior” and “exterior” “railway stations” of the “PKC” transport vesicle machinery) /1, 41/ to work after 2nd obligatory activation in cytoplasm. Other mRNA (coding nonstocked proteins) (belonging to the cytoplasmic ncnskeletal ribosomes or ER membrane ribosomes-/Refs.1/) are transported by other ways such as one with help of hnRNPs /99/ (heterogenous nuclear ribonuclear proteins).

Evidently, the end of the signal, with an absence of the stocked proteins, must lead to the apoptosis/Part VII, Y.Z.-Application FR-95-11550-retired/, because of an impossibility to begin a functioning of the “heart” “PKC” transporting vesicles without a stock needed for the construction of the machinery without the transport, non functioning yet.

§7.General Position of Such Signal: PM→Nucleus in Cell Signaling.

What is the place of the above signal spreading from PM to ER and nucleus in the complex (generally) signal with a number of other events as, for instance, a creation of the protein networks near the PM or the integrin interactions? Evidently, almost independently of such PM events (except the sub-pathway of Na⁺/H⁺ exchanger activation), the activation of the immediate-early genes must take place as well a liberation of the stocked proteins (on, also activated by CL, ribosomes) for immediate functioning of the “PKC” transporting vesicle machinery—the “heart” of any complete cell signal /1/. A creation of the specific for each signal network of the membranous proteins is necessary mainly for a creation of the “outer” “railway station” (including the intergin's sub-station /1, Y.Z. Application FR-95-11550 retired/) for the “PKC” vesicle transporting cycle, Including the melting of PM with help of the homologous intercarbohydrate (locally dehydrating) interactions /1, Part II/.

Until yesterday, the world scientific knowledge about the signal spreading to the nucleus reduced at the end only to a statement of the phosphorylation /100, FIG. 1.1.108/. The signal where, for instance, the protein kinases change (at once) a conductivity of the ionic channels (as after the adrenaline action on β-adrenergic receptors in heart muscle cells) without a creation of the “PKC” vesicle transport machinery are only degenerated noncomplete signals /1/. Today this knowledge is reduced to the (in reality) false picture of “SH2-SH3- . . . ” protein network spreading until . . . the nucleus (evidently due to unrealistic scale of the molecules in the cell) /101-FIG. 15-34- “an informative pathway . . . until nucleus”/.

So the vectorial transport due to the “PKC” vesicle transporting machinery until ER and nucleus (FIG. 5) together with the described here special signal spreading (FIG. 4) turn the Page of Scientific Imaginations.

AII. Primary Nucleotide Structures of Important Proteins and Mechanism of Process of Special Universal TRansduction of the Signal from Plasma Membrane to Nucleus.

It is shown that the signal from the PM to the nucleus conducts (by simple diffusion of small ions) to the cytoplasmic activation of the unusual protease Cathepsin L (CL) (so called also Major excretion protein /MEP/). This CL liberates different proteins from a particular attachment to the rRNA of the ribosomes by the limited specific proteolysis of the end part of the molecule. The available already results clearly indicate that all these proteins belongs to the family that intensifies the cycle of the signal transporting “PKC” vesicles with their own specific vesicular PKC /2/. But, clearly, not all proteins participate in this signaling as one can judge, for instance, from the persistent absence of an attachment to the advanced (in growth cone) rnRNA of the neurotransmitter receptors /3/. So by this limited proteolysis, the 1st “railway” of the “PKC” transporting vesicles is constructed from the simply detached stocked proteins without protein synthesis. Moreover, the detached (in cytoplasm) early nuclear factors go into nucleus (like NF-kB /4/) to continue the complex signal network.

Fortunately, the great number of the primary nucleotide structures confirms the main stocking character of Universal signal transduction. Moreover quite different details of a presence and localisatlons of Gly—Arg like peptides in important proteins permit to prove very important characteristics of the general functioning of these proteins.

§1.Presence of Gly—Arg Sequences in Other Signaling Proteins of “PKC” Vesicle Transport Machinery and Their Isoforms.

A perfect correlation between GR sequences at the protein ends of a number of the “PKC” transport vesicle machinery proteins with a number of functional facts in spite of a number of errors in DNA sequencing data /5/ is very convincing. Such propeptide with GR sequences in the CL molecule (˜75 amino acids) (justly until GR group) or in the N-part of the α-chain of casein kinase II (CK-II (vesicular /2/) justly has an extended conformation /6, 7/ obviously facilitating the limited proteolysis of the CL and of the N-end of α-chain.

Cathepsin L. The always present GR groups in CL determine, logically, an attachment of these molecules to the ribosomes at stocking /Refs.8-11/. Moreover, it is very visible that this GR group determines also a place of the proteolysis at the same nearby area that can be at very (!) different bonds there (Gln—Glu and Met—Leu) producing in vitro ˜30 kDa single chains (from 39 kDa) /12/. But In the case of human CL, this GR group is situated the 51 amino acids downstream /Refs.8/ and consequently there is a global creation of the smaller 25 kDa mature form already after the net cell spreading (signal!) (without “processing defect” /13/ taking place due to, logically, thelysosornal digestion with, for instance Cathepsin D) Such excitnig dependence of the proteolysis region on the GR peptide location is well confirmed in the case of the trematode CL (38 kDa) /10/ where the GR group is closer to C-end and consequently the size of. the proteolysed. form (31 kDa) justly correspond to such area. Also in the 3 lobster CL sequences, the position of the GR peptide is similar and much closer to the N-end and, once more, the molecular weight of the proteolysed form, equal to 28 kDa (with 217 amino acids from 322), corresponds to such proteolysis location (especially without posttranslational modifications de facto) /11/. But a destination of CL could be different (Part AII, §3) because the several of its mRNA have (long) poly(A)⁺ tail whereas several others do not have it /8, 9/, that is underlined by a presence of 2 alternative mRNA splicings with a difference in 739 nucleotides between 2 their polyadenylation signals and with difference in ˜500 nucleotides between lengths of their mRNA, that must mean a difference in 239 nucleotides between their poly(A)⁺ tails /14/ (splicing with 1st polyadenylation signal is longer).

The c-jun. It is known that the nuclear factor c-jun is also bound in cytoplasm “in waiting” the signal /15/ being activated with the calpain (CL-like protease) cleavage /16/. However, it has such sequence in the N-end of the proprotein if to continue the 5′-nucleotide sequence upstream until the stop codon, always present /17,Refs.18/. But also, there are the sequences of c-jun without such GR peptides before the stop codon (at 5′- end) /18,Refs.19,20/. Logically, the forms of c-jun with GR groups usually serve for the slower tournover with an attachment in the nucleus preproribosome and stocking in cytoplasm and ones without GR groups for more rapid (only cytoplasmic) tournover during already “PKC” vesicle transport cycle work.

The P53. Also the important regulatory protein p53 always has (25 cases!) several such groups at its C-end and never at its N-end including the sequences upstream “open reading frame” (“ORP”) until the existing “5′”-stop codon /Refs.21; 22, 23/. Justly, this p53 protein binds the. mdm-2 protein with such N-end, making an interaction with the rRNA with its free C-end with GR peptides (including final covalent binding by its extreme amino. acid to 5.8S rRNA) /24/:. Evidently, the p53 liberation takes place also with proteolysis of its C-end part in the cytoplasm.

The c-fos. And the c-fos protooncogene (3 cases) does. not have the group GR upstream of “ORF” until “5′” stop condon (Refs.19). But it has the “strong” amino acid group GRRG together with GGR between amino acids 117 and 127 permitting the rapid limited proteolysis with the calpain (CL-like) 1/25/, reflecting. a presence of several homologous proteins (mol.weight 46, 35 and 30 kDa) beginning from amino acid 127 of c-fos /Refs.25/ with logical liberation of the c-fos stocked proteins (like c-jun) without protein synthesis after the signal /Refs.26/. This cleaved region is situated before functioning regions of c-fos /Refs-27/. An absence of the GR peptides in ALL. c-fos proteins coincides with beginning of the functional domain (Al) with HOBI and HOB2 sequences already from amino acid 5 from “ORF” that is also coincides with difference in the c-f os proteolysis, characterized by the cleavage of a big piece of the c-fos nonfunctional N-end /Refs.27/. The (clear) proteolysis of the c-jun already with the 26S proteosome dependent manner 128/ (once more In difference with c-fos) confirms such convergences.

The protein kinase C. A similar situation takes place with the PKC. ALL its spread forms (α, β, γ, δ, ε, ζ) are an object of the limited proteolysis in the N-end of these molecules where there are justly the GR groups (especially very strong “sure” RRGR group) /Refs.29/ generating the active (generally fully) catalitic C-end fragment (so called protein kinase M) /Refs.29/. Moreover, justly the calpaln (CL-kind!) cleaves the PKC /Refs.29/ and after the signals like the phorbol, Ca and Ca ionophore, almost all PKC molecules are rapidly cleaved in situ /Refs.29;30/ and are freely translocated (as it is well known) to the PM /Refs.29/. A convergence is striking. A futher proteolysis serves for the long desensitization of the PKC /31/. If to take into consideration that the PKC is very stable /Refs.29/, one can understand that this slower tournover is not a narrow part of the “PKC” transporting vesicle cycle and there is no special need for more rapidly activated forms without the ADMA attachment as in the case of the c-jun and also this explains an absence of the oncogene forms of PKC /32/. Justly a presence of such c-fos oncogene forms, in spite of their common activation with sole CL proteolysis with the large cleaved N-part, is convergently explained by a weak stability of the c-fos,/25,Refs.19/.

The phosphatidylinositol-specific Phospholipase C (PI-PLC). In difference with PKC, the PI-PLC of all main different forms have already a number of GR peptides in different parts of “ORF” of these molecules /Refs.33;34, 35/ and justly in last case, the limited proteolysis (by calpain included) produces the subunits with different mol. weight from different parts of the initial molecule (with X and Y catalytic parts), where, after a strong interaction between X and Y parts (that, logically, also protects them), an activity of the PI-PLC increases (!) /36-38/. The. signal activation of the cell through the CL-like calpain (CL in reality-Y.Z.) limited proteolysis of the PI-PLC /refs.36; 39 with Refs/ (proved with leupeptine that is also an inhibitor of the cathepsins /401) as well a movement of PI-PLC from cytoplasm to particular fractions with activation /41/ confirm such good convergent data. A presence of the main forms also in the nucleus confirms once more these data /Refs.42, 43/.

The nucleolin, CK-II and bFGF. The important nuclear protein nucleolin makes interactions by its N-domain with chromatin and justly has no these GR peptides there although it has very intensive patches of these peptides at C-end which, in this case, justly interact with preproribosomes /44/.

The CK-II (vesicular /2/) has the two subunit types: α and β. All chains have the constant “good” GRG site in the proximal part of its N-end (although without GR in “prepeptide” until always present stop codon) /45, 46/. However, there is an intensive presence of the β-subunits without these groups in their propeptides and sequences /Refs.45;46/. The insistent limited proteolysis of the α-subunits (with strong mol.weight change) and its absence at the β-subunit during purification /Refs.47;48/ confirms these convergent data. Also the decreased observed mol.weight (in relation to calculated from primary nucleotide sequence) of the α-subunits and the increased one of the β-subunits /46/ confirm such proteolysis. An addition of the leupeptine (justly inhibitor of the papain-like proteases /40/) during a purification clearly stopped the proteolysis /48/.

The α (α′) subunits make a complex with the spread β-forms (that are without GR-groups). In an absence of the α-subunits, the β-subunits do not go into the nucleus /49/, that must mean in reality that justly α (and α′) subunits, (but not β) have the sites of an attachment to the ribosomes and moreover have the strong (it means special) interactions with intracellular components ( nucleolin particularly) /50, 51/ (parts of preproribosomal complex). A bridging role of the (only) β-subunits making interactions between them as well with the α (α′) subunits /52/ where moreover the β-β interaction is negatively regulated with the special β-subunit phosphorylation /52/ confirm this mechanism of the CK-II formation where it is the α (α′) subunits that direct an attachment of the β-chains in the nucleus preproribosomes. Such interaction with the β-chains saves the α-chains from the proteolysis /53/. Evidently, the α and α′ subunits can undergo the N-end proteolysis by CL during the signal. The very special structure of the very proteolysable propeptide in the α′-subunits (many Arg and Pro) /Refs.47;48/, logically can permit their CL proteolysis (especially with limited quantity of free β-subunits) before entering into the nucleus for αβ complex, giving the special form of the CK-II without entering into the nucleus with a much quicker formation /54/ during the intensive “PKC” transporting vesicle work /2/. A synthesis of this form during neural development (in neural growth cone: it means with intensive signal transport) /55/ confirms these data. A role of CK-II in the protective phosphorylation of the stocked proteins /2, 56, 57/ is confirmed also by a very large number of phosphorylated proteins /58/, especially that are stocked and participate in the “PKC” transport vesicle cycle.

In the case of the bFGF (basic fibroblast growth factor), where a long sequence until “5′” stop codon contains a number of strong patches conducting to the proteolysis in, justly (!) several N-end sites /Refs.59/, the stocking (firstly in nuclear preproribosomes) takes place only with a help of the ADMA /60/. Exceptionally, the short mature forms of bFGF (18 kDa) have two (D)GR groups (at No. 46-48 and 88-90) that normally should not be proteolysed because (exceptionally) of their special protection by heparin /Refs.59/, similar to heparin sulfate, that should be present in the area of the bFGF synthesis /Refs.2; Refs.59/. A high heparin binding by the peptides 27-69 and 70-153 from bFGF sequence /61/ and the specific inhibition of the bFGF mytosis by the RGDS peptides /82/ confirm such interactions because the heparan sulfate is very important for such bFGF signal /Refs.2,Refs.59/. Unlikely, the special form aFGF (acidic FGF) has the “5′” stop codon immediately upstream of the short form and does not have any GR peptide /Refs.59/. The synthesis of this acidic FGF form in hepatocytes during the signal for their movement /63 with Refs/ (with intensive continuous signals) confirms such exception.

Steroid receptors. In the case of the steroid receptor as retinoic acid receptor (RAR) there are the 3 main forms (α, β, γ), where there is no at all the GR peptides in the “ORF”, but upstream of the “ORF” (until “5′” stop codon) there are the strong GR(P) peptides in RAR-γ and there is no such peptides in the α and β forms /Refs.64/. This rnust reflect (as in the case of c-jun) an easiness of the α and β form liberation (short tournover) during the signal and a longer tournover of the γ-form (together with the intersignal stock). A dominant presence of the RAR-A forms (without GR peptides) in poly(A)⁺ mRNA family /65/ (it logically means the destination to the exterior “railway station”- Annex AII) confirms this conclusion.

A new location of the estrogen receptor at the cone outgrowth /66/ reflects a similar situation. There is also an often presence of the long poly(A)⁺ mRNA of this receptor /Refs.66/ that argues for the special destination for a rapid use of such forms of this receptor (AII, §3). -The form of this receptor is without the GR groups in “ORF” (and upstream of it) and visibly with especially long poly(A)⁺/67/, that justly could reflect a presence of the form longer than 67 kDa (unproteolysable by CL) justly during the cancer (with intensive signals). The patch with 2 close GR peptides in the N-part of this receptor /Refs.64/ well sensitizes the receptor to be proteolysed after stocking: during the protein synthesis in vitro (with rabbit reticulocyte lysate) there is, logically, the intensive limited proteolysis-in situ of the entire receptor form (64 kDa) into 46-kDa. /88/ and also in situ, such proteolysis takes place during the cancer cellular cycles (presence of 43 and 35 kDa main forms) /69, 70/ and the mild rapid limited proteolysis in vitro takes place also (˜35 kDa) /Refs.69/ as consequences of these positions of the GR groups.

The permanent activating non-negligeable (it means with big piece cleaved) proteolysis of such important molecules as PKC, PI-PLC or steroid receptors confirms an importance of such Universal mechanism.

The β and γ actins. In the case of nonmuscle actins there are the two forms β and γ that are almost identical /71/. One can see a presence of the GR peptide upstream of the “ORF” until “85” stop codon in the γ and β nonmuscle actins /72 with Refs./. The synthesis of the β-actin takes place in the neuron cone /73/ or near the PM /74/ (due to the long poly(A)⁺ tail) in difference with the γ-actin /71/. The actins have the GR group at 36-37 positions of the DNAase I binding loop /75/ that (also exceptionally) is not proteolysed (although the region is sensible) /Refs.76;77/ because they are protected by the Mg²⁺ ions /76/ with the actin C-end help /77/. Logically, as in the case of bFGF, the functional sense of such GR presence, connected, for instance with negative nuclear interactions (as control of nuclear channels or chromosomal transition activity) must be present /78, 79/.

The Rel complexes. In the case of Rel (3 proteins in complex), the situation, is similar. The stocked complex p105(p5o)/p65(ReIA)/IkB-α was already described /1/. The generally present forms p100/p52, RelB and IkB-β must represent another (nonstocked) cytoplasrnic version of the Rel complexes. The p100 does not have the GR sequence upstream of the NFkB2 “ORF” until the close stop TAA codon /80, 81/. The ReiBs have the GR peptide near the N-end of the protein structure with an unusually high number of the prolines near the arginine /82/ making them very proteolysable. The special character of the DNA interaction with the human RelB (“I-Rel”-“inhibitory”) (logically not yet proteolysed), due to a larger sequence upstream of the “ORF” until the stop codon /82/, confirms the limited proteolysis presence. And the IkB-β (mol.weight 43 kOa) is easily proteolysed (logically) at the N-end where the resulting p40 form /83, 84/ reflects the easily proteolysable sequence presence. These forms are present due to the complex of the three molecules where the quick proteolysis (from N-end) must take place at the unification of these molecules. The fact, that the IkB-β makes (at once) the complex after the synthesis in cytoplasm, being without the phosphoprotection /85/ in difference with the IkB-α (from long waiting Rel complexes), confirms this general conclusion. The p105/p50, p65(ReIA) and IkB-α (only) protein presence in the stocked cytoplasmic complexes /86/ and the serum stimulation of the RelB concentration increase /82,Refs.87/ also confims it. The p100(p52)/RelB/IkB-β complex specificity is also visible from following data: the IkB-α Interacts weakly with RelB, the p52/RelB does not associate with IkB-α and the IkB-α affinity (A) to the different complexes represents the following relations: A(p50/RelA)>A(p50/RelB)>A(p52/RelB) IRefs.87/.

§2.Stop Codons Stabilize the Stocking.

The stop codon permanent presence at the continuation upstream of the “ORF” 5′ end is very remarkable (except, naturally, cases without the well given nucleotide sequence). This persistent fact justly coincides well with the ribosomal pose at the stop codon that “can be a major factor In stabilizing downstream regions of mRNA” /88, 89/. The fact that these “5′” stop codons usually do not have the uridine in the position “-3” upstream of the stop codons in the polar difference with the normal “3′” stop codons /90/ confirms their new function. Evidently, it is the very strong convergent information of the reality of the long cytoplasmic poses of the corresponding mRNAs in G0 and after G1. The new perfect confirmation, one can see from the “abnormally effective recognition of NON-AUG codons” (as beginning) upstream of the AUG codon that “is strongly Influenced by Mg²⁺ levels” /91 with Refs/. And justly, the protein synthesis takes place at the time and in the region of the action of the “PKC” transporting vesicles, that transport the Mg²⁺ and Ca²⁺ ions, making the increase of their local concentrations /2/.

So with the signal beginning, the translation from the propeptide sequence must take place. And justly, the protooncogens, all of which must participate in the transport vesicle machinery functioning /2/, are often translated from the NON-AUG codons /92/. Logically, in the G0 state, when the local Mg concentration (due to the chaotic zero level of the “PKC” vesicle transport, evidently diminished) is less strong, the synthesis from the downstream AUG codon can, logically, take place /5/ with a shorter protein sequence.

Moreover, such molecules often have the GC rich 5′-mRNA /93, 94/ potentializing more often an appearance of the arginine, glycine, alanine and proline, justly necessary for an above functioning including a facilitation of the proteolysis with the arginine and praline. On the other hand, such GC- rich sequence diminishes a spead of a translation of these molecules because justly an increase of a spead of the “PKC” vesicle transporting machinery Universally leads to all existing forms of cancer /2/.

§3.The mRNA as Guide for its Ribosomes.

As one can see, the concretisation of these very universal “GR”-rRNA interactions for different proteins gives very particular cases. There is an another superposition that decides a localisation of these proteins with the ribosomes that synthesize them. This localization is directed by the 3′ region of the mRNA translated by the corresponding ribosomes /Refs.95;Refs.96/. For instance, its AU rich elements, a structure of which, clearly, is not yet universalized /Refs.96;97/, promote an attachment of the ribosomes to the cytoskeleton /96/ (part of “PKC” transport vesicle machinery /2/). An another very important signal is the polyadenylation that directs the mRNA-ribosomes-proteins to an other compartmentalisation: growth cone of developing neurites or cell periphery /74, 98, 99/ during “PKC” transporting vesicle cycle. The synthesis of the hormone mRNA with a large poly(A)⁺ (with the signal!) /100, 101 with Refs./ with its compartmentalisation at the. cone /102/ (or near the “PKC” transport vesicle PM “railway station”) illustrates this process. A presence of the regulated (justly by differentiation) mRNA with higher mol.weight, going to the axon cone /103/, confirms a regulation. of a compartmentalisation by mRNA polyadenylation. The biological sense of a synthesis of GAP-43, tubulin, β-actin already at the cone /73/ is obvious as well a more effective secretion of the hormone near already the subsurface “railway station”.

Part X. Practical Consequences of Parts VII-X and Annex.

The irresistible proofs of the mechanisms of the process of the interactions between the envelope viral molecules and CD4 receptors (and their Generalisation for All Virology), clearly done in Part VII (and also VIII-X) with help of the establishment of very Universal Laws of Protein Foldings, Functionings and Recognitions, and the consecutive functioning of the Universal Du-2T- like proteins (Parts VII-X), also have led to the (also) Universal preparations against all Viruses (and also other parasites: bacteria, protozoans, mashrooms). An existence of such Uttles Proteins is 100% in the case of Immunoglobulins G (IgG) and the molecules of MHC class I (Parts VIII, IX). Their dissociation is obligatory during action of All Viruses (and other above parasites). So a simple introduction (in organism) of these Universal Du-2T-like proteins immediately after invasion of such or such (anyl) virus, bacterium, protozoan or champignon will diminish (cancel) the actions of the parasites from beginning. One can also pulverise such Du-2T-like proteins (Parts VII-IX) of the virus and other parasites of the plants for agriculture. An obtaining of each such Du-2T peptide is elementary/par exemple, according works of Dudich E. I., Dudich I. V. and Timofeev V. P. (Part II- Refs.2, 4): one diminishes the pH with “Du-2T” dissociation and makes the elementary separation sur filters Amicon/ and their sequencing (˜15 amino acids) is too routine and automatic (and evidently, their synthesis is also automatic). Evidently, one can also determine the end amino acids of such peptides and to see its sequence according the published prinriary (nucleotides included) structures of the preprotein.

Evidently, after resolution (Part X) of the mechanism of the process of formation of the priones (correct Nobel Prise of 1997, unfinished at all), the end of such diseases as Mad Cow or Creutzfeldt-Jacob is evident. One must only introduce the corresponding Du-2T-like proteins in organism of sick man or animal.

Evidently, the synthesis of the functional proteins (like enzymes, the high affinity molecules as antibodies, the receptors, the nuclear factors, the repressors, the activators) can be really done in vitro with help of the molecules as chaperons (with their precise and successive addings in dissociating the previous chaperon with, par example, acid solutions), like the peptidyl prolylisomerase (PPI) and like protein disulfideisomerase (PDI) and also the Du-2T like proteins (Part VII).

Even the proteins with new sequences, synthesized artificially, must have the parts of the primary structure, necessaire for foldings (S—S bonds, two nearby prolines, attachment sites for homologous carbohydrates, prosequences (N- or C-) for Petites Proteins (Peptides) Du-2T- like. (Parts VII-IX).

Evidently, the synthesis of the proteins in vitro (even on the artificial surfaces) with help of the ribosomes (obtained from cell cytoplasm), always recycled, must be corrected according Annex AI: particularly, the proteolysis, activating the proribosomes by Cathepsin L (MEP) must be obligatory.

We have seen that the cell apoptosis is characterised by depletion of the stocking of the proteins of the “PKC” transport vesicle cycle (Annex AI, §6) and also of the substances (like phosphorylated derivatives of phosphatidylinositol),necessary. for the functioning of these transport vesicles /1/, at time quand this transport cycle does not function. Evidently, the cell transport cannot be reconstructed and the cell must die. This exhaustion of the phosphorylated derivatives of phosphatidylinositol at the moment of stopping of the functioning of the cycle of the transport vesiciles is made, principally, by the vesicular PI-PLC, well exceptionally proved in jungles of researches, very bad conducted (Part VII and also /1/ and FR-95-11550, retired). The irreversible death (because of the oxygen absence, already 5 minutes. after the clinical death, is due to cells of the brain. These new.characteristics, justly, explian such phonomenon, because, without functioning of the mitochondria, there is no transport of the “PKC” and synaptic vesicles /1/. And after long stopping of the cycle hear the “Railway station Calcisomes” /1/, the PI-PLC-α (vesicular) hydrolyses (without mesure) the phosphorylated derivatives of the phosphatidylinositol. Consequently, the substances cannot already arrive to the “railway”. The neuron terminals are, justly, the most sensible because of the length of the axonal transport (with “PKC” vesicles). The utilisation of the inositol, phosphatidylinositol (PI), PI-P (phosphate), PIP₂ (PI disphosphate) (or their derivatives less hydrolysable and their lysoderivatives) must permit to potentiate the defense of the functioning of the cells in the states of the clinical death and coma.

One can see the conscience as the reflection of the cyclic system of the neurons with chaotic transport CYCLES of the “PKC” and synaptic (“PKC”-like) vesicles (analogy with autoactivatlons of the heart rhythm: heart cells) /1, PCT/FR98/02334-retired/. The “order” to sleep must be done due to the reversible (until some critic level of irreversible death) decrease of the level of the transporting (and conductingi /1/) vesicle cycles intensity due to the decrease of the concentration level of the PIP₂ (serving for functioning of the transporting and conduicting “PKC” vesicle cycle /1/) (CONSCIENCE, at the end, as the closed network of such cycle currents In the neuronal, also closed, cycles, Justly), where such quantity of the phosphorylated derivatives diminishes during the working day and the action of the hypnotics must contain a prbperty to cut the spontanous excitations of the “PKC” and synaptic vesicles (“CLOSED NETWORK OF CONSCIENCE”). The cyanate, in stopping the respiration, stops the cycle of the synaptic (“PKC”-like) and “PKC” transporting and conducting vesicles and their long way provision support. But with weaker dozes, it is the hypnotic with perfect advantage: due to its small size, it leaves the organism the most easily (the best of any other existing hypnotics). Such basic obligatory “reversibility” /1/, due to a weaker dozes, is well known for hypnotics.

IIIA. Perfect Proofs of Existence of Transporting Vesicle Cycle with Muscle Physiology.

This (Part I,§1;Annex AI) “PKC” transporting vesicle machinery cycle, so important for the cell signalling (Annex AI) and its “extreme” expression in cell mobility (Part I,§1) is well visible during the skeletal and cardiac muscle functioning /1,FIG. 29,28/. Moreover, its establishment can be supported by the extraordinary convergences between the global physiological muscle data (Application PCT/FR98/02334 :“Process of scientific reanimation with generalisation of the scientific modifications of the organ functioning. Agony and epilepsy with treatment”, retired). It is well known that the motoneurons make the action potential “TRAIN” in response to the voluntary stimulation or sense receptor action /1, 2/, but nobody knows the goal! My profound advances in the CONDUCTIVITY process along the skeletal and cardiac muscle /1-FIG. 29,28/ permits to resolve this curious question. After the increased amplitude of the motoneuron synaptic current there is, justly, the repetitive dischargments where these action potentials are followed by the prolonged AHP (after hyperpolarisation) (lasting 50-200 msec) /3/. The frequences in these “trains” are from tens Hz until 200 Hz /3-5,1/. Also, there is the cortical pyramidal neurons with the bursts with groups of the action potentials wherein each burst (“train”) contain many pulsations with at least 150-300 Hz! These “trains” are repeated with their frequence of 5-15 Hz /6,p.313/. But one does not register the action potentials with the too strong frequence, obtaining them In situ as the sole large action potential /Refs.7,p.57/.

With the discovery of the muscle conductivity process /1-FIG. 29/, where the signal passes by the chains of the Na/K/Ca and Ca-K(Ca) channels, intermittent along the muscle (FAST one), one explains the necessity of the signal TRAINS FOR the ENTIRE muscle contraction, because during one sole pulsation (action potential) (with Ca liberation) there is.only the small sarcomer displacement. Moreover, during waiting the 2nd pulsation, the slow skeletal muscles are excited nonsynchronically (it means permanently with justly, the numerous innervations along the skeletal muscle). It means there are the bridges of the myosin heads on the actine during the muscular contraction. It is the solution (confirming the muscular contraction mechanism /1-FIG. 29/) of the remarked (but not resolved) Haxley's paradox /8/. The following pulsations achieve the skeletic muscle contraction. The cardiac muscle has the similar conductivity mechanism /1,FIG. 29/ and must also have the signal “trains” for Its contraction. But there is no slow muscles in the cardiac musculature to keep the appIled force. So the frequence in the pulsation “train” must be increased. So such uncomprehensible, permanent presence of the “TRAIN” of the action potentials for muscle contractions converges perfectly with the obligation of the large number of the cycles of the transporting permanent vesicles to make the complete large contraction discreetly “little by little” (anyway, if to increase the distance between the action potentials in “train”) by the fast skeletal fibers whereas the slow fibers (always present in whole muscle) enable to keep the contraction level between the action potentials. So the proofs of such functional transport vesicle cycle in muscle are impressive.

### References.

Part I. Patent Publication No. FR-2644347 (1990). 2.Montagnier L. et al Patent Publication No. US-4708818 (1987). 3.Zagyansky Y. International Patent Publication No. WO 88/06837 (1988) (PCT). 4.Zagyansky Y. European Patent Publication No. EP-0347536 (1989). 5.Cold Spring Harb. Symp.Quant Biol. 60,249,1955. 6.lmmunomethods 4,229,1994. 7.AdvJmmunol: 45,1,1989. 8.Virus Res. 32,227,1994. 9.Coll.Physicians Lond. 26,204,1992. 10.J.immunol. 156,1284,1996. 11.FASEB J. 5,2391, -1991. 12.Virology 147,231,1986. 13.Am.J.Pathol. 145,428,1994. 14.Semin. Cell Biol. 5,149,1994. 15 .J.Cell Biol.123,977,1993. I .Nature 302,532,1983. 17.Microbiol.Rev. 60,386,1996. 18.Adv.Exp.Med.Biol. 374,47,1995. 19.Blood 75,1215,1990. 20.Clin.Exp.lmmunol.95,442,1994. 21.Adv.Exp.Med.Biol. 374,211,1995. 22.J.Med.Primatol. 24,94,1995. 23.Clin.lnfect.Dis. 19,500,1994. 24.Curr.Top.Microbiol .immunol. 183,1,1993. 25.Virus Res. 32,111,1994. 26.Microbiol.Rev. 57,183,1993. 27.Adv.Exp.Med.Biol. 374,27,1995. 28.Curr.Opin.Immunol. 7,24,1995. 29.Nature 343,659,1990. 30.APMIS 100,465,.1992. 31.FEBS Lett. 386,239;1996. 32.Blood 88,1215,1996. 33.Ann.lntern.Med. 121,100,1994. 34.Transfusion 36,681,1996. 35.J.lnfect.Dis. 168,1260,1993. 36.J.Biol.Chem.269,13811,1994. 37.J.Immunol. 156,3693,1996. 38.Immunol.Today 16,569,1995. 39.Adv.Exp.Med.Biol. 374,183,1995. 40.Curr.Top.Microbiol.Immunol. 205,47,1996. 41.1mmunol. Today 14,215,1993. 42.J.Immunol. 157,1625,1996. 43.Blbod 88,3792,1g96. 44.Immunol.Today 16,297,1995. 45.Blood 83,2191,1994. 46.Curr.Top.Microbiol. 205,137,1996. 47.J.Immunol. 158,1157,1997. 48.Mol.Immunol. 22,161,1985. 49.Annu.Rev. Immunol. 12,457,1994. 50.Structure 2,59,1994. 51.J.Immunol. 158,216,1997. 52.AIDS Res.Hum.Retroviruses 8,1581,1992. 53.Dudich El & Dudich IV Mol.Immunol.20,1267,1983. 54.Dudich El, Dudich IV & Timofeev VP Mol.Immunol. 20,1273,1983. 55.Biochemistry 14,3778,1975. 56.Cell 61,243,1990. 57.Adv.Immunol. 24,37,1976. 58.Dev.Neurosci. 11,276,1989. 59.Philos.Trans.R.Soc.Lond.B Biol.Sci. 327,171,1990. 60.Clin.Exp.Rheumatol. 6,355,1988. 61.Respiration 61,Suppl.1,8,1994. 62.AIDS Res.Hum.Retroviruses 9,1277,1993. 63.Annu.Rev.Immunol. 12,593,1994. 64.Nature 384,529,1996. 65.Mol.Med. 3,23,1997. 66.Biomed.Pharmacother. 48,445,1994. 67.Am.J.Pathol. 148,1367,1996. 68.,J.Clin.Invest. 97,2722,1996. 69. lrnmunol.Lett. 53,105,1996. 70.Immunol.Cell Biol. 74,513,1996. 71.Annu.Rev.Immunol. 8,139,1990. 72.Zagyansky Y. -Part II- here. 73.AIDS Res.Hum.Retrovir. 8,1581,1992. 74.Virology 205,34,1994. 75.Protein Eng. 9,671,1996. 76.Cell 61,243,1990. 77.EMBO J. 8,2889,1989.

Part II. 1.Zagyansky Y. Part I. 2.Curr.Opin.Struct.Biol. 3,393,1993. 3.Curr.Opin.Struct.Biol. 3,701,1993. 4.J.Biol.Chem. 264,2385,1989. 5.J.Struct.Biol. 109,201,1992. 6.Glycoblology 3,429,1993. 7.J.CIin.lnvest. 91,379,1993. 8.J.Immunol. 149,1957,1992. 9.Nature 372,150,1994. 10.J.Biol.Chem. 270,27127,1995. 11.“Encyclopedia of Molecular Biology” Ed.Kendrew J. (Blackwell Science, Oxford),p.439. 12.Nature 385,557,1997. 13.Glycobiology 4,837,1994. 14.Matrix 13,215,1993. 15.Arch.Biochem.Biophys. 304,65,1993. 16.J.Biol.Chem. 261,13186,1986. 17.J.Biol.Chem. 271,17006,1996. 18.Glycobiology 8,635,1996. 19.Adv.Immunol. 52,425,1992. 20.Arch.Virol. 141,291,1996. 21.Arch.Virol. 126,11,1992. 22.Glycobiology 7,129,1997: 23.Proc.Natl.Acad.Sci.USA 89,663,1992. 24.Immunogenetics 29,281,1989. 25.Proc.Natl,Acad.Sci.USA 86,8521,1989. 26.Proc.Natl.Acad.Sci.USA 87,7000,1990. 27.Blochem.Blophys.Res.Comm. 110,424,1983. 28.J.Biol.Chem. 269,4473,1994. 29.J.Exp.Med. 181,877,1995. 30.J.Biol.Chem. 269,20807,1994. 31.Eur.J.Biochem. 238,511,1996. 32.Trends Blochem.Sci. 15,291,1990. 34.Biochem.Biophys.Res.Comm. 178,1194,1991. 34.Glycobiology 4,413,1994. 35.J.Biol.Chem. 271,70.81,1996. 36.J.Infect.Dis. 170,51,1994. 37.Adv.Immunol. 47,187,1989. 38.Annu.Rev.Blochem. 57,785,1988. 39.Annu.Rev.Immunol. 11,613,1993. 40.J.Immunol. 142,.1177,1989. 41.J.Virol. 83,2270,1989. 42.AIDS Res.Hum.Retroviruses 11,481,1995. 43.AIDS Res.Hum.Retroviruses 12,1129,1996. 44.Cell Immunol. 172,235,1996. 45.Eur.J.Immunol. 20,215,1990. 46.Cell 47,333,1986. 47;Zagyansky Y. European Patent Publication. EP-0347536 (1989). 48.Microbiol.Rev. 57,183,1993. 49.AIDS Res.Hum.Retroviruses 7,45,1991. 50.Eur.J.Biochem. 192,523,1990. 51 .J.Biol.Cheim. 263,11760,1988. 52.Virology 180,853,1991. 53.Arch.Biochem.Biophys. 290,248,1991. 54.Immunol.Today 13,301,1992. 55.J.Virol.68,463,1994. 56.Molec.Immunol. 31,569,1994. 57.Glycobiology 3,429,1993. 58.Proc.Natl.Acad.Sci.USA 84,1649,1987. 59.Proc.Natl.Acad.Sci.USA 84,7644,1987. 60.Eur.J.Immunol. 23,179,1993. 61.J.Immunol. 158,216,1997. 62.Arch.Virol.141,291,1996. 63.J.Virol. 62,1016,1988. 64.J.Virol. 158,216,1998. 65.Immunol.Lett. 47,141,1995. 66.Zagyansky Y. Patent Publication. No. FR-2644347 (1990). 67.Biochim.Biophys.Acta 1161,194,1993. 68.J.Am.Soc.Nephrol. 7,955,1996. 69.Contrib.Nephrol. 111,73,1,989. 70.Biochem.J. 286,131,1992. 71.AIDS Res.Hum.Retroviruses 6,813,1990. 72.Nature Struct.Biol. 3,596,1996. 73.J.Virol. 67,3639,1993. 74.Proteins 25,1,1996. 75.Clin.Exp.Immunol. 80,420,1990. 76.J.Virol. 67,584,1993. 77.Virology 180,1,1991. 78.Can.J.Chem. 36,563,1958. 79.Adv.Carbohydr.Chem. 6,108,1951. 80.Philos.Trans.R.Soc.Lond.B Biol .Sci. 278,33,1977. 81.Carbohydr.Res. 91,101,1981. 82.J.Solut.Chem. 13,209,1984. 83.Trends -Bidchem.Sci. 14,272,1989. 84.“Physiological Regulation of Membrance Fluidity” Eds. Aloia R C, Curtain C C & Gordon L M (Liss Inc N.Y.), p.75. 85.J.Cell.Biol. 51,536,1971. 86.J.BioI.Chem. 256,13039,1981. 87.Zagyansky Y. International Patent Publication NO. WO 88/06837 (1988). 88.Trends Biochem.Sci. 3,110,1978. 89.Bioessays 12,173,1990. 90.J.Biol.Chem. 272,24780,1997. 91.Biochem.J. 308,801,1995. 92.Genomics 39,192,1997. 93.J.Bioi.Chem. 272,9709,1997. 94.J.Biol.Chem. 272,13843,1997. 95.Curr.Struct.Biol. .6,663,1996. 96.Genomics 21,18,1994. 97.Biochem.Soc.Transac. 25,871,1997. 98.Eur.J.Biochem. 217,645,1993. 99.Biochem.J. 326,911,1997. 100.J.Biol.Chem. 270,24166,1995. 101.J.Biol.Chem. 268,18960,1993.

Part.III. 1.Rev.Med.Virol. 3,173,1993. 2.J.Acquir.Immune Defic.Syndr. 3,965,1990. 3.J. Virol. 69,8164,1995. 4.J.lnvest.Dernatol. 99,99S,1992. 5.J.Gen.Virol. 77,1083,1996. 6. Virology180,229,1991. 7.J.Biol.Chem. 271,5393,1996. 8.J.Virol. 63,5194,1989. g.J.Virol. 66,305,1992. 10.Science 254,423,1991 g 11.J.Virol. 67,6863,1993. 12.Cell 61,213,1990. 13.J.Virol.66,1717 ,1992. 14.J.Leukoc.Biol. 56,289,1994. 15.J.Virol. 70,8701,1996. 16.1mmunol.Lett. 51,3,1986. 17.AIDS 10,31,1996. 1 8.Marm,:Genome 5,688,1994. 19.Virology 189,344,1992. 20.AIDS 5,617,1991. 21.Adv.Canc.Res. 51,183,1988. 22.,J.Virol. 68,6644,1994. 23.Virology 205,470,1994. 124.AIDS Res.Hum.Retroviruses 12,1443,1996. 25.J.Vlrol. 69,321 6,1995. 26.J.Virol.64,2421,1990. 27.J.Virol. 69,376,1995. 28.Nature 343,85,1990. 29.Zagyansky Y. Part I- here. 30.JAMA J.Am.Med.Assoc. 256,2365,1986. 31.AIDS 5,639,1991. 32.J.Virol. 67,4972,1993. 33.J.Virol. 64,4169,1990. 34.EMBO J. 8,2653,1989., 35.Semin;Virol.5,71,1994. 36.J.Virol. 7.1,95,1997. 37.J.Vlrol. 70,8187,1996. 38.AIDS 3,475,1989. 39.J.Acquir.Immune. Defic.Syndr.Hum.Retrovirol. 10, S15,1995. 40.Proc.Natl.Acad.Sci.USA 80,339,1983. 41.Proc .Natl.Acad.Sci.USA 83,7246, 1986.; 42.J.Cell Biol. 126,1221,1994. 43.J.Neurosci. 18,1346,1996. 44.J.Neurosci. 14,1130,1994. 45.Eur.J.Biochem. 63,166,1994. 46.Biochem.Soc.Transact. 24,521,1996. 47.J.Virol. 64,2265,1990. .48.J.Virol. 64,5306,1990. 49.Virology 178,435,1990. 50.J.Virol. 69,4060,1995. 51.Mol.Cell.Biol. 12 ,1375,1992. 52.J.Virol. 70,3645,1996. 53.J.Virol. 68,5433,1994. 54.Zagyansky Y. Patent Publication No. FR-2644347 (1990). 55.Ann.Intern.Med. 124,984,1996. 56.Mathe G. “SIDA. Sceaux, Sex, Science” (La Coutellerie, Paris, 1994). 57.AIDS .3,665,1989. 58.Lancet 348,239,1996. 59.Science 260,1273,1993. 60.New Engl.J.Med. 322,941,1990. 61-New Engl.J.Med. 326,437,1992. 62.Lancet 343,1354,1994. 63.Lancet 343,871,1994. 64.AIDS 9,1165,1995. 65.AIDS 4,221,1990. 66.J.Exp.Med. 169,933,1989. 67.Gen.Pharmacol. 26,1469,1995. 68.AIDS 8,F1,1994. 69.lnt.J.Clin.Pharmacol.Ther. 33,85,1995. 70.Mlcrobiol.Rev. 57,183,1993. 71.Biochem.Soc.Transac. 19,255,1991. 72.Am.J.Physiol. 253,C707,1987. 73.Cell Tiss.Res. 240,433,1985. 74.Allergy 50,157,1995. 75.J.Allergy Clin.Immunol. 89,68,1992. 76.J.lnfect.Dis. 164,1,1991. 77.Lancet 345,1342,1995. 78.Science 287,1820,1995. 79.J.Gen.Vlrol. 77,2969,1996. 80.AIDS 10 (Suppl.A) ,S33,1996. 81.Science 270,988,1995. 82.J.Virol. 69,5048,1995. 83.J.Virol. 69,4053,1995. 84.J.Biol.Chem. 271,12762,1996. 85.Curr.Top.Microbiol.Immunol. 188,185,1994. 86.Biochem.Biophys.Res.Comm. 178,1194,1993. 87.Zagyansky Y.- Part V. 88.J.Biol.Regul.Hemost.Agents 6,15,1992. 89.J.Med.Virol. 35,65,1991. 90.Adv.Immunol. 52,425,1992. 91.J.Virol. 66,5937,1992. 92.J.Biol.Chem. 271,16753,1996. 93.Cell 82,665,1995. 94.Virology 230,82,1997. 95.J.Virol. 70,6701,1996. 96.Int.Immunol. 4,1113,1992. 97.J.Immunol. 151,2928,1993. 98.Cell 65,651,1991. 99.“Encyclopedy of Molecular Biology” Ed.Kendrew J. (Blackwell Science, Oxford), p.515. 100.Zagyansky Y. Part II. 101.Brit.Med.J. 298,713,1989. 102.J.Med.Virol. 28,209,1989. 103.Am.J.Publ.Health 78,1535,1988. 104.J.Med.Virol. 35,14,1991. 105.N.Engl.Med.J. 320,1458,1989. 106.Lancet 348,1347,1996. 107.Cell Mol.Biol. 41,345,1995. 1 08J.Acquir.Immune. Defic.Syndr. 6,303,1993. 109.J.Acquir.Immune Defic.Syndr. 2,77,1989. 11 O.AIDS 7,1363,1993. 111 .J.Virol. 63,4277,1989. 112.Immunol.Lett. 51,35,1998. 113.J.Exp.Med. 180;769, 1994. 114.J.Immunol.-.162, 2308,1 999, - 1 5.J.Infebt:Dis. 178,T799,f 99&8 f1:6.Natm.ed; 2,405,1996. 119.Proc.Natl.Acad.Sci.USA 87,3972,1990. 120.Scand.J.Immunol. (Suppl.) 36(11),81,1992. 121.J.Med.Primatol. 25,158,1996. 122.Nature 391,397,1998.

Part IV. Br.Med.J. 301,1362,1990. 2.AIDS 1,105,1987. 3.N.Engl.J.Med. 321,947,1989. 4.Am.J.Epidemiol. 132,203,1990. 5.N.Engl.J.Med.- 316,61,1987. 6.Zagyansky Y. Part I- here. 7.Zagyansky Y. Patent Publication No. FR-2644347 (1990). 8.AIDS 7,51,1993. 9.Lancet 338,1159,1991. 10.Pharmacol.Ther. 55,201,1992.11.J.lnter.Am.Med.Health Assoc. 1,1,1992. 12.Nature 362,103,1993. 13.J.Dermatol. 32,784,1993. 14.Mayo Clin.Proc. 67,1089,1992. 15.Arch.Dermatol. 126,539,1990. 16.Busvine J R ”Diseases transmission by insects” (Springer-Verlay, Berlin,1989). 17.J.Infect.Dis. 160,970,1989. 1 8.AIDS 7,341,1993. 19.Vir.Res. 32,111,1994. 20.Arch.Dermatol. 127,206,19.91. 21.South.Med.J. 87,352,1994. 22.Dermatology 192,143,1996. 23.Immunopharmacol. 17,103,1995. 24.Zagyansky Y. Part III. 25.J.Acqulr.Immune Defic.Syndr. 5,251,1992. 26.J.Immunol. 148,2709,1992. 27.S.Afr.Med.J. 66,806,1984. 28.Clin.Exp.Immunol. 71,13,1988. 29.Immunology 74,567,1991. 30.Crit.Rev.Oncol.Hematol.1 9,47,1995. 31.Arch.Intern.med. 148,2296,1988. 32.J.Acquir.Immune Defic.Syndr. 1,367,1988. 33.J.Am.Acad.Derrnatol. 29,269.,1993. 34.!Infect.Dis.Clin.North Am. 8,583,1994. 35.Clin.Infect.Dis. 17,724,1993. 36.Annu.Rev.Microbiol. 48,687,1994. 37.J.Acquir.Immune Deftc.Syndr. 5,782,1992. 38.Antivir.Chem.Chemother. 6,Suppl.1,17,1995. 39.J.lnfect.Dis. 172,672,1995. 40.Am.J.Respir.Crit.Care Med. 151,129,1995. 41.Rev.Med.Virol. 6,163,1996. 42.Immunology 83,507,1994. 43.Proc.Natl.Acad.Sci.USA 92,8378,1995. 44.Exp.Neurol. 134,214,1995. 45.Clin.Immunol. 95,886,1995. 45a.AIDS 11,949,1997. 46.Zagyansky Y. Part II. 47.Adv.Immunol. 52,425,1992. 48.AIDS 7,S37,1993. 49.J.Virol. 64,3779,1990. 50.J.Med.Primatol. 22,194,1993. 51.1mmunol.Allergy Clin.North Am. 15,205,1995. 52.J.Pediatr. 123,929,1993. 53.AIDS 7,Suppl.2,S49,1993. 54.Zagyansky Y. Part III. 55.Zagyansky Y. Part I. 56.J.Acquir.Immune Defic.Syndr. 14,368,1997. 57.J.lnfect.Dis. 174,866,1996. 58.J.Exp.Med. 178,1459,1993. 59.Lancet i,949,1985. 60.J.Child Neurol. 8,378,1993. 61.AIDS Res.Hum.Retroviruses 9,1277,1993. 62.N.Eng.J.Med. 330,308,1994. 63.AIDS 7,1683,1993. 64.J.Immunol. 154,433,1955. 65.Pediatr.Res. 33,469,1993. 66.Antimicrob.Agents Chemother. 39,125,1995. 67.Arch.Dis.Childhood 76,215,1997. 68.Pediatr.lnfect.Dis.J. 15,891,1996. 69.Pediatrics 89,297,1992. 70.Acta Pedlatr. 83,173,1994. 71.AIDS 7,SuppI.,S91,1993. .72.Pediatr.Res.lnfect.Dis.J. 11,639,1992. 73.Pediatrics 89,123,1992. 74.AIDS 3,577,1989. 75.Clin.Microbiol. 5,238,1992. 76.J.Thorac.Cardiovasc.Surg. 101,909,1991. 77.Bioessays 12,173,1990. 78.J.Immunoi. 157,4963,1996. 79.Annu.Rev.Biochem. 57,785,1988. 80.Proc.Natl.Acad.Sci.USA 80,6632,1983. 81.J.BIol.Chem. 271,12762,1996. 82.AIDS 7,1139,1993. 83.Pedlatr.Res. 40,597,1996. 84.Clin.Infect.Dis. 15,502,1992. 85.Pediatr.Infect.Dis:J. 11,974,1992. 86.Lancet 340,585,1992. 87.Trop.Doct. 26,100,1996. 88.Acta. Vet.Scand. 37,31,1996. 89.J.Acquir.Immune Defic.Syndr. 8,188,1995. 90.N.Engl.J.Med. 323,531,1990. 91.Lancet 343,191,1994. 92.Lancet ii,916,1987. 93.Arch.Blochem.Biophys. 290,248,1991. 94.J.Leukoc.Biol. 56,218,1994. 95.Microbiol.Rev. 59,481,1995. 96.lnt.J.Epidemiol. 21,387,1992. 97.Br.Med.J. 311,156,1995. 98.1nfect.1mmun. 64,2821,1996. 99.Ann.N.Y.Acad.Sci. 177,156,1971. 100.“Textbook of AIDS Medicine” Eds.Broder S., Mergan tC & Bolognesi D. (Williams & Wilkins, Baltimore, 1994),p.21. 101.J.Leukoc.Biol. 56,247,1994. 102.Semin.Immunol. 5,187,1993.103.AIDS 5,1433,1991. 104.J.Med.Virol. 28,81,1989. 105.Nature 391,240,1998. 106.Science 274,1543,1996. 107.AIDS 10,1057,1996. 108.J.lnfect.Dis. 1B6,738,1992. i09.Science 279,327,1998.

Part V. 1.Zagyansky Y. Patent Publication No. FR-2644247 (1990). 2.Zagyansky Y. Part III. 3.zagyansky Y. Part I. 4.Zagyansky Y. Part IV. 5.J.Immunopharmacol. 17,75,1995. 6.Ann.N.Y.Acad.Sci. 540,649,1988. 7.J.Virol. 62,3073,1988. 8.J.Virol. 64,1437,1990. 9.J.infect.Dis. 170,51,1994. 10.Zagyansky Y. Part 11. 11.1mmunol.Lett. 51,107,1996. 12.J.Virol. 66,3398,1992. 13.J.Virol. 70,3741.,1996. 14.J.Antimicrob.Chemother. 37,Suppl.B,185,1996. 15.Am.J.Vet.Res. 47,537,.1986. 16.J.Virol. 64,2396,1990. 17.J.Virol. 69,3704,1995. 18.AIDS Res.Hum.Retroviruses 12,437,1996. 1s.Zagyansky Y. Europeen Patent Publication EP-0347536 (1989). 20.Progr.Polym-Sci. 4,1,1975. 21.lnt.J.Clin.Lab.Res. 26,124,1996. 22.AIDS Res. Hum.Retroviruses 13,677,1997. 23.Biochemistry 33,6974,1994. 24.Biochem.Biophys.Res.Comm. 209,850,1995. 25.J.Acquir.Immune Defic.Syndr. 3,965,1990. 26.Immunol.Today 13,301,1992. 27.Microbiol.Rev. 57,183,1993. 28.AIDS 3,475,1989. 29.Curr.Opin.1mmunol. 2,414,1990. 30.Microbiol.Rev. 57,183,1993. 31.Ann.N.Y.Acad.Sci. 727,50,1994. 32.Virology 186,261,1992. 33.Nature 337,368,1989. 34.Proc.Nati.Acad.Sci.USA 86,4287,1989. 35.AIDS 1 o(Suppl .A),S33, 1996. 36.J.Acquir.Immune Defic.Syndr. 3,319,1990. 37.J.Med.Primatol. 22,154,1993. 38.Immunol.Lett. 51,107,1996. 39.AIDS 10,689,1996. 40.J.Trop.Pediatr.42,116,1996 41.Science 268,1612,1995. 42.Annu.Rev.Med. 41,331,1990. 43.Clin.Microbiol.Rev. 10,86,1997. 44.J.Infect.Dis. 158,124,1988. 45.Transplant.Proc. 20, Suppl .1,661,1988. 46.Semin.Pediatr.Surg. 2,218,1993. 47.Nippon Rinsho 54,1529,1996. 48.Oncogene 13,427,1996. 49.Annu.Rev.Immunol. 12, 593,1994. 50.1mmunol.Cell.Biol. 74,513,1996. 51.Virology 225,111,1996. 52.J.lnfect.Dis. 174,1098,1996. 53.Immunology 82,410,1994. 54.J.Infect.Dis.Suppl. 99,30,1995. 55.Scand.J.lnfect.Dis.Suppl. 99,34,1995. 56.Transplantation 61,1757,1996. 57.J.Virol. 71,1521,1997. 58.Scand.J.lnfect-Dls.Suppl. 99,43,1995. 59.Virology 197,143,1993. 60.Cell 76,301,1994. 61.“Fields Virology” Eds.Fields B N, Knipe D M & Howley P M (Lippincott-Raven Publishers, Philadelphia, 1996),p.2221. 62.J.Virol. 67,2209,1993. 63.“Fields Virology” Eds.Fields B N, Knipe D M & Howley P M (Lippincott-Raven Publishers, Philadelphia, 1996),p.2343. 64.J.Virol. 67,2918,1993. 65. U.lnfect.Dis. 165,994,1992. 66.“Fields Virology” Eds.Fields B N et al (Uppincott-Raven. Publishers, Philadelphia, 1996), p.2231. 67.“Fields Virology” Eds.Fields B N et al (Lippincott-Raven Publishers, Philadelphia), p.2297. 68.Virology 224,214,1996. 69.Virology 179,487,1990. 70.J.lnterferon Res. 14,319,1994. 71.Pharmacol.Ther. 65,415,1995. 72.AIDS Res.Hum.Retroviruses 12,1273,1996. 73.Virology 221,1,1996. 74.J.Gen.Virol. 75,1211,1994. 75.Annu.Rev.Cell Biol. 8,365,1992. 76.J.Virol. 68,2830,1994. 77.Am.J.Pathol. 134,223,1989. 78.Adv.NeuroImmunol. 5,327,1995. 79.Clin.lnfect.Dis. 19,500,1994. 80.Vet.Microbiol. 55,277,1997. 81.Proc.Natl.Acad.Sci;USA 91,1932,1994. 82.Microb.Pathog. 14,275,1993. 83.N.Engl.J.Med. 322,1648,1990. 84.Nature 362,103,1993. 85.lnt.Arch.Allergy Immunol. 103,128,1994. 86.Zagyansky Y. International Patent Publication No. WO 99/56288 (04/11i99) and WO 00/52989 (14.09.00.) “Elnstein-Bohr End: New Atomic Scale Physics, Electric field: neutrinos and electrons In conversions, perpetual motion, development: seisms, exstinguished volcans, created islands, Big Bang Energy” —full text in: http://pctgazette.wipo.int 87.lnfect.Control.Hosp.Epidemiol. 17,532,1996.

Part VI. 1.“Immunochemistry-Labfax” Eds.Kerr M A & Thorpe R. (Biosci. Publishers, Oxford, 1994). 2.Harlow E. & Lane D. “Anticorps. Un Manuel de Laboratoire” (Prodel, Paris, 1991). 3.Methods in Molecular Biology: vol.51 “Antibody engineering protocols”. 4.J.Am.Chem.Soc. 116,6508,1994.

Part VII. 1.Proc.Natl.Acad.Sci.USA 95,13233,1998. 2.J.Gen.Virol. 77,1837,1996. 3.Science 229,1402,1985. 4.Science 228,593,1985. 5.Eur.J.Biochem. 260,482,1999. 6.J.Neurosci.Res. 33,639,1992. 7.Bioessays 12,173,1990; Bioessays 12,223,1990. 8.J.Biol.Chem. 268,4922,1993. 9.Exp.Eye Res. 47,53,1988. 10.Biochim.Biophys.Acta 986,106,1989. 11.Protein Sci. 3,1953,1994. 12.Int.J.Pharrnaceut. 134,193,1996. 13.EXS 77,41,1996. 14.Biochem.Cell.Biol. 73,123,1995. 15.Plant Mol.Biol. 32,191,1996. 16.FASEB J. 9,1559,1995. 17.Cold Spring Harb.Symp.Quant.Biol. 60,405,1995. 18.EMBO J. 15,6921,1996. 19.Eur.J.Biochem. 244,334,1997. 20.J.Biochem. 114,763,1993. 21.FEBS Lett. 387,27,1996. 22.Biochemistry 33,3229,1994. 23.J.Cell.Biol. 136,555,1997. 2⁴.Nature 385,557,1987. 25.J.Biol.Chem. 269,1744,1994. 26.Experimentalia 50,1012,1994. 27.Nature 370,373,1994. 28.Curr.Opin.Struct.Biol. 6,663,1996. 29.J.Biol.Chem. 270,1705,1995. 30.J.Cell Biol. 126,41,1994. 31.“Encyclopedy of Molecular Biology” Ed.Kendrew J. (Blackwell Science, Oxford, 1994).32.Acta Neurobiol.Experim. 56,287,1996. 33.J.Biol.Chem. 267,16396,1992. 34.J.Biol.Chem. 272,27116,1997. 35.J.Cell Biol. 133,1177,1996. 36.Biochem.J. 301,917,1994. 37.Biochem.J. 301,918,1994. 38.Biochem.J. 301,919,1994. 39.lnt.J.Cancer 56,286,1994. 40.lnter.J.Biochem. 25,157,1993. 41.EMBO J. 12,3143,1993. 42.Trends Biochem.Sci. 19,331,1994. 43.J.Biol.Chem. 265,1094,1990. 44.Mol.Cell.Biol. 12,4601,1992. 45.FEBS Lett. 372,210,1995. 46.EXS 77,97,1996. 47.Gene 164,377, 1995. 48.Proc.Natl .Acad.Sci.USA 88,4453,1991. 49.J.Biol.Chem. 268,7728,1993, 50.Arch.Biochem.Biophys. 323,397,1995. 5t.J.Biol.Chem. 267,3553,1992. 52.Biochim.Biophys.Acta 1309,253,1996. 53.Nature 334,268,1988. 54.Meth.Enzymol. 197,526,1991. 55.Arch.Biochem.Biophys. 308,454,1994. 56. Curr.Opin.Cell.Biol. 8,499,1996. 57.Zagyansky Y. “New Created Fundamental Chapters of Cellular and Intercellular Molecular Biology in 2050 (with top medical applications)”, ISBN:2-9502914-8-1, Paris, 1995. Very detailed Abstract in: Lancet vol.346, No.8986 (18/111/95), p:Classified 2 (No. D710). (PCT/FR94/01159, retired). Also European Patent Publication EP-0347536 (1989). 58.J.Biol.Chem. 266,20337,1991. 59.EMBO J. 14,4688,1995. 60.Bio-chem.J. 316,623,1996. 61.Proc.Natl.Acad.Sci.USA 90,3973,1993. 62.J.Biol.Chem. 271, 15874,1996. 63.EMBO J. 16,3506,1997. 64.ibid 16,3494,1997. Zagyansky Y. Eur.Pat.Publ. No.EP-0347536 (1989). 66.Eur.J.Biochem. 216,689,1993. 67.Annu.Rev.Biophys.Blol.Struc. 22,123,1993. 68.J.Cell.Physiol. 125,251,1985. 69.Curr.Opin.Struct.biol. 3,104,1t993. 70.Physiol.Rev. 76,631,1996. 71.J.Biol.Chem. 240,1882,1965. 72.J.Biol .Chem. 241,5408,1966. 73.Arch.Biochem.Biophys. 125,715,1968. 74.Trehds Biochem.Sci. 22,87,1997. 75.Mol.Biochem.Parasitol. 79,13,1996. 76.Annu.Rev.Pharrnacol .Toxicol. 37,297,1997. 77.Biochemistry 36,3600,1997. 78.Genes Develop. 10,1491,1996. 79.J.Biol.Chem. 270,16198,1995. 80.Biochem.Blophys.Res.Comm. 191,860,1993. 81.Oncogene 7,1207,i992. 82.Oncogene 7,1625,1992. 83.J.Biol.Chem. 267,2902,1992. 84.J.Biol.Chem. 272,16224,1997. 85.Biochemistry 34,8123,1995. 86.J.Cell Sci. 108,2037,1995. 87.Mol.Endocrinol. 5,1215,1991. 88.EMBO J. 13,4278,1994. 89.FEBS Lett. 400,136,1997. 90.Transplantation 53,460,1992. 91.Adv.Prot.Chem. 44,25,1993. 92.Tumer-man L. & Zagyansky Y. Theses of International Congress IUPAC,Riga,1970 “Multiglobular structure of proteins”, Egorov A M Vestnik Moskovskogo Universiteta ser.6 Biological, U.S. Pat. No. 6,123,1970 “About multinuclear structure of globular proteins”. 93.J.Biol.Chem. 268,15611,1993. 94.Biochem.J. 291,723,1993. 95.J.Virol. 70,778,1996. 96.J.Virol. 68,5063,1994. 97.J.: BioloChem. 269,7006,1994. 98.J.Cell.Biochem. 65,574,1997; 99.Biochem.J. 302,641,1994. 100.Blochemistry 36,7776,1997. 101.Mol.Cell.Biol. 15,3917,1995. 102.1nt.J.Cancer 54,153,1993. 103.J.Biol.Chem. 266,2499,1991. 104.J.Biol.Chem. 266,4686,1991. 1 05.J.Biol.Chem. 270,10650,1995. 106.Proc.NatI .Acad.Sci .USA 90,7074,1993. 107.Biochemistry 33,8912,1994.

Part VIII. 1.Dudich El, Franek F & Nezlin RS FEBS Lett. 89,89,1977. 2.Dudich El & Dudich IV Mol.Immunol. 20,1267,1983. 3.J.Bioi.Chem. 257,277,1982. 4.Mol.Immunol. 20,1273,1983. 5.Zagyansky Y. International Patent Publication (PCT) No. WO 88/06837 (1988). 6.Zagyansky Y. “New Created Fundamental Chapters of Cellular and Intercellular Molecular Biology in 2050 (with top medical applications)“, ISBN:2-9502914-8-1, Paris,1995. Very detailed Abstract in: Lancet, vol.346,No. 8986 (18/11/95),p.Classified 2 (No.D710). (PCTIFR94/t159). 7.J.Cell Biol. 111,829,1990. 8.Mol.Immunol. 27,623,1990. 9.EMBO J. 11,1573,1992. 10.lImmunity 2,629,1995. 11.J.Biol.Chem. 272,3117,1997. 12.“Fundamental Immunology” Ed.Paul W E (Raven Press, N.Y.,1993,3d Ed.). 13.J.Mol.Biol. 248,190,1995. 14.Hibridoma 10,651,1991. 15.J.Biol.Chem. 272,22556,1997. 16 .Biochem.J. 317,659,1996. 17.J.Biol.Chem. 269,14290,1994. 18.J.Biol:Chem. 265,1980,1990. 19.Nature 386,506,1997. 20.J.Exp.Med. 170,1175,1989. 21.J.Biol.Chem. 268,5866,1993. 22.J.Biol.Chem. 269,28997,1994.

Part IX. 1.Annu.Rev.Immunol. 14,369,1996. 2.1mmunol.Res. 14,200,1995. 3.Immunol.Res. 16,243,1997. 4.Cell 89,167,1997. 5.1mmunogenetics 45,285,1997. 6.Immunogenetics 45,209,1997. 7.Nature 329,506,1987. 8.Nature 384,134,1996. 9.Annu.Rev.Immunol. 8,139,1990. 10.Trends Biochem.Sci. 21,427,1996. 11.EMBO J. 7,3745,1988. 12.Zagyansky Y. International Patent. Publication WO. 88/06837 (1988). 13.Immunol.Rev. 101,149,1988. 14.Annu.Rev.Immunol. 5,503,1987. 15.J.Exp.Med. 180,433,1994. 16:.J.Immunol. 156,35,1996. 17.EMBO J. 16,4205,1997. 18.J.Immunol. 158,1201,1997. f9.Zagyansky Y. “New Created Fundamental Chapters of Cellular and Intercellular Molecular Biology in 2050 (with top medical applications)”, ISBN:2-9502914-8-1, Paris,1995. Very detailed Abstract:. Lancet 346, No.8986,p.Classified 2 (D710). 19a.J.Immunol. 156,3275,1996. 19b. ibid 150,2831,1993. 19c.ibid 136,6,1986. 19d.1mmunology 64,175,1988. 20.Current Biol. 7,R299,1997. 21.Eur.J.immunol. 26,544,1996. 22.Eur.J.Immunol. 25,3270,1995. 23.Eur.J.Immunol. 24,1066,1994. 24.Nature 345,41,1990. 25.Nature 387,630,1987. 28.Cell 40,237,1985. 27.J.Immunol. 152,397,1994. 28.1mmunogenetics 38,27,1993. 29.J.Immunol. 158,2236,1997. 30.J.Exp.Med. 178,2035,1993. 31.1mmunity 4,565,1996. 32.1mmunol.Today 18,299,1997. 33.Eur.J.Immunol. .23,1173,1993. 34.J.Cell Sci. 100,329,1991. 35.lntern.Immunol. 8,1947,1996. 36.Zagyansky Y. European Patent Publication No. EP-0347536 (1989). 37.EMBO J. 10,893,1991.

Part X. 1.Cold Spring Harbor Symp.Quant.Biol. 61,495,1996. 2.Curr.Opin.Struct.Biol. 7,53,1997. 3.Cold Spring Harb.Syrnp.Quant.Biol. 61,511,1996. 4.Biochemistry 31,5043,1992. 5.Biochemistry 36,3543,1997. 6.J.Virol. 70,4724,1996. 7.European Patent Publication No.EP-0347536 (1989). 8.Trends Biochem.Sci. 21,482,1996. 9.Zagyansky V. “New Created Fundamental Chapters of Cellular and Intercellular Molecular Biology in 2050 (with top medical applications)”, ISBN:2-9502914-8-1, Paris, 1995 Very detailed Abstract in: Lancet vol.346,No.8986 (18111/95), p.Classified 2 (No. D710). (PCT/FR94/1159). 1 O.Biochemistry 28,8380,1989. 11 .Brit.Med.Bull. 49,873,1993. 12.Blochem.Soc.Transac. 25,307S,-1997. 13.Biochem.Soc.Transac. 22,163,1994. 14.J.Virol. 67,643,1993. 1 5.Proc.Natl.Acad.Sci.USA 93,14945,1996. 16.Cold Spring Harb.Symp.Quant.Biol. 61,473,1996. 17.FASEB J. 5,2799,1991. 18.Neurology 47,741,1996. 19.Cold Spring Harb.Symp.Quant.Biol. 61,531,1996. 20.Biochem.J. 320,563,1996. 21.Cell 59,847,1989. 22.Biochemistry 32,1991,1993. 23.Proc.Natl.Acad.Sci.USA 92,3923,1995. 23.Proc.Natl.Acad.Scl.USA 92,3923,1995. 1 24.J.Gen.Virol. 70,3371,1989. 25.Gene 184,125,1997. 26.Genetika 33,610,1997. 27.Neuron 7,59,1991. 28.Curr.Top.Microbiol.Immunol. 172,57,1991. 29.Trends Gen. 12,467,1996.

Annex.IA. 1.Zagyansky Y. “New Created Fundamental Chapters of Cellular and Intercellular Molecular Biology in 2050 (with top medical applications)” ISBN: 2-9502914-8-1, Paris,1995. Very detailed Abstract is published In: Lancet, vol.346,No.8986 (18/11195),p.Classified 2 (No. D71 0) (PCT/FR94/1159). 2.Progr.Biophys.Mol.Biol. 24,177,1972. 3.J.Physiol. 227,855,1972. 4.Science 247,858,1990. 5.Cell Calcium 16,247,1994. B.Cell Calcium 17,165,1995. 7.Eur.J.Biochem. 174,3,1988. 8.J.Membr.Blol. 135,93,1993. 9.Mol.Pharmacol. 48,995,1995. 10.J.Biol.Chem. 270,29209,1995. 11.Am.J.Physiol. 246,R409,1984. 12.Biochim.Biophys.Acta 988,73,1989. 13.J.Cell Sci. 101,55,1992. 14.Trends Cell Biol. 6,168,1996. 15.Proc.Natl.Acad.Sci.USA 83,357,1986. 16.Biochem.Biophys.Res.Comm. 131,402,1985. 17.Biochem.J. 248,449,1987. 18.Science 265,615,1994. 1 9.J.Biol.Chem. 268,10501,1993. 20.Proc.Natl.Acad.Sci.USA 92,5282,1995. 21.J.Cell.Physiol. 144,108,1990. 22.J.Cell.Biol. 129,233,1995. 23.Cell Regul. 2,87,1991. 24.J.Biol.Chem. 261,9167,1986. 25.J.Bibl.Chem. 260,14304,1985. 26.J.Biol.Chem. 266,2007,1991. 27.Arch.Biochem.Biophys. 297,169,1992. 28.Biochem.Biophys.Res.Comm. 43,644,1971. 29.Mol.Biol.Cell 6,S1890,1995. 30.J.Gen.Physiol. 57,244,1971. 31.Arch.Biochern.Biophys. 172,627,1976. 32.Metabolism 28,801,1979. 33.Am.J.Physiol. 269,H1 522,1995. 34.Annu.Rev.Physiol. 57,737,1995. 35,Nuci.Acid Res. 12,3025,1984. 36.Exp.Cell Res. 203,259,1992. 37.J.Virol. 69,6751,1995. 38.lnt.J.Cancer 60,495,1995. 39.J.Biol.Cherh. 264,7377,1989. 40.J.Cell.Biochehn. 40,31,1989. 41.Annex IIA here. 42.Endocrlne J. 2,249,1994. 43.J.Mol.Biol. 210,541,1989. 44.J.Cell Sci. 107,105,1994. 45.J.neurosci. 1B,3265,1996. 46.Proc.Natl.Acad.Sci.USA 91,10766,1994. 47.J.Neurosci. 14,1130,1994. 48.J.Neurosci. 16,1400,1996. 49.Mol.Cell Neurosci. 3,1221,1994. 50.J.Cell Biol. 126,1221,1994. 5t.Cell Biol. 126,1211,1994. 52.Recept.Channels 1,11,1993. 53.J.Cell Biol. 126,1221,1994. 55.Biochem.J. 298,143,1994. 56.Mol.Cell.Neurosci. 7,289,1996. 57.J.Biol.Chem. 271,12585,1996. 58.Biochim.Biophys.Acta 1248,11,1995. 59.Proc.NatI .Acad.Sci.USA 93,6043,1996. 60.J.Cel l Biol. 100,535,1985. 61 .Biochem.J. 201,189,1982. 62.Curr.Top.Cell Regul. 22,71,1983. 63.Biochem.Soc.Trans. 19,255,1991. 64.J.Biol.Chem. 265,13650,1990. 65.Biochemistry 32,375,1993. 66.J.Cell Biochem. 40,31,1989. 67.J.Biol.Chem. 264,20487,1989. 68.Biochem.J. 272,39,1990. 69.Arch.Biochem.Biophys. 312,101,1994. 70.FASEB J. 10,A1400,1996. 71.Exp.Cell Res. 212,338,1994. 72.1nfect.1mmun. 63,911,1995. 72a.Cancer J. 8,72,1995. 75b.J.Biochem. 119,719,1996. 73.Adv.Cancer Res. 66,255,1995. 74.Nucl.Acid Res. 23,2328,1995. 75.Cell 53,211,1988. 76.J.Biol.Chem. 271,6084,1996. 77.Cell 46,705,1986. 78.J.Cell Biol. 120,577,1993. 79.J.Biol.Chem. 270,9,1995. 80.EMBO J. 13,5433,1994. 81 .Proc.Nati.Acad.Sci.USA 92,552,1995. 82.Cell 79,13,1994. 83.Mol.Cell.Biol. 15,1302,1995. 84.Mol.Cell.Biol. 15,1294,1995. 85.Nucl.Acid Res. 21,5059,1993. 86.Adv.Immunol. 58,1,1995. 87.Mol.Cell.Blol. 16,2248,1996. 88.DNA Cell Biol. 11,523,1992. 89.Genomics 30,493,1995. 90.J.Biol.Chem. 268,10501,1993. 91.Trends Gen. 6,390,1990. 92.J.Cell Sci. 99,465,1991. 93.Cell 56,379,1989. 94.Science 271,1513,1996. 95.Proc.Natl.Acad.Sci.USA 73,4531,1976. 96.Exp.Cell Res. 178,264,1988. 97.Nucl.Acid Res. 12,3025,1984. 98.Dev.Biol. 173,420,1996. 99.Trends Cell Biol. 6,290,1996. 100.“Hormones” Eds.BauIleu E E & Kelly P A (Herman, publishers in art and in science, Paris,1990). 101.“Molecular Biology of the Cell” Eds. Alberts B., Bray D., Lewis J., Raff M., Roberts K. & Watson J D (Garland Publishing Inc, N.Y.,3d Ed.,1994).

Annex IIA. 1.Zagyansky Y. Annex IA- here. 2.Zagyansky Y. “New Created Fundamental Chapters of Cellular and Intercellular Molecular Biology (with top medical applications)”, ISBN: 2-9502914-8-1, Paris, 1995. Very detailed Abstract in: Lancet, vol.346,No.8986 (18/11/95), p.Classified 2 (N-D710), (PCT/FR94/1159). 3.Proc.Natl.Acad.Sci.USA 91,10766,1994. 4.Cell 53,.211,1988. 5.Mamrnal.Genonie 7,563,1996. 6.EMBO J. 15,5492,1996. 7.Eur.J.Biochem. 196,159,1991. 8.J.Cell.Biochem. 40,31,1989. 9.Mol.Reprod.Dev. 40,146,1995. 10.Mol-Cell.Parasitol. 73,7,1995. ii.FEBS Lett. 292,115,1991. 12.FEBS Lett. 352,281,1994. 13.Biochem.J. 257,493,1989. 14.Mol.Endocrinol. 5,1789,1991. 15.EMBO J. 11,1479,1992. 16.FEBS Lett. 287,57,1991. 17.Oncogene 3,659,1998. 18.Nucl.Acid Res. 18,3400,1990. 19.Protein Profile 2,105,1995. 20.J.Cell.Biochem. 57,479,1995. 21.J.Mol.Biol. 260,623,1996. 22.Nucl.Acid Res. 16,11383, 1988. 23.DNA Sequence 5,255,1995. 24.Mol.Cell.Biol. 14,7414,1994. 25.Biochem.J. 313,245,1996. 26.Crit.Rev.Oncog. 1,65,1989. 27.Protein Profile 1,123,1994. 28.J.Biol.Chem. 270,11623,1995. 29.Annu.Rev.Biochem. 58,31,1989. 30.FEBS Lett. 350,223,1994. 31.J.Biol.Chem. 270,19651,1995. 32.FEBS Lett. 350,223,1994. 33.Biochem.J. 288,1,1992. 34.Zool.Studies 34,141,1995. 35.J.Biol.Chem. 268,21318,1993. 36.FEBS Lett. 340,185,1994. 37.Eur.J.Biochem. 213,339,1993. 38.Biochem.J. 302,503,1994. 39.Biochem.Biophys.Acta 1223,84,1994. 40.Protein Sci. 5,752,1996. 41.Biochem.J. 305,745,1995. 42.Protein Sci. 5,680,1996. 43.J.Biol.Chem. 271,355,1996. 44.Cell.Mol.Neurobiol. 14,407,1994. 45.Pharm.Theurap. 59,1,1993. 46.Mol.Cell.Biol.Res. 40,481,1994. 47.Annu.Rev.Biochem. 56,567,1987. 48.J.Biol.Chem. 256,8955,1981. 49.J.Cell.Mol.Neurobiol. 14,407,1994. . 50.Mol.Cell.Blochem. 129,77,1993. 51.J.Biol.Chem. 271,15662,1996. 52.J.Mol.Biol. 253,703,1995. 53.Eur.J.Biochem. 204,293,1992. 54.J.Biol.Chem. 270,7501,1995. 55.Cell.Mol.Biol.Ref. 40,581,1994. 56.Prostate 24,101,1994. 57.Am.J.Physiol.- Cell Physiol. 40,C304,1996. 58.FASEB J. 9,313,1995. 59.Adv.Cancer Res. 59,1.15,1992. 60.Mol.Biol.Cell 7,1249,1996. 61.Ann.N.Y.Acad.Sci. 638,98,1991. 62.Ann.N.Y.Acad.Sci. 638,361,1991. 63.Oncogene 5,755,1990. 64.Protein Profile 2,1173,1995. 65.Eur.J.Blochem. 232,425,1995. 66.Endocrine J. 2,249,1994. 67.EMBO J. 5,891,1986. 68.Proc.Natl.Acad.Sci.USA 82,7889,1995. 69.Breast Canc.Res.Treat. 40,231,1996. 70.Cancer Biochem.Biophys. 15,67,1995. 71.Curr.Opin.Cell.Biol. 5,48,1993. 72.Mol.Cell.Biol. 8,1775,1988. 73.J.Neurosci. 14,1130,1994. 74.J.CelI Biol. 126,1221,1994. 75.Differentiation 14,123,1979. 78.Biochem.J. 316,713,1996. 77.BiophysJ. 74,1914,1996. 78.Arn.J.Physiol.-Cell Physiol. 39,Cl532,1996. 79.Biol.Cell 84,139,1995. 80.Mol.Cell.Biol. 12,685,1992. 81.Nucl.Acid.Res. 23,2388,1995. 82.Mol.Cell.Biol. 13,1572,1993. 83.Gen.Devel. 5,1464,1991. 84.Cell 61, 255,1990. 85.Mol.Cell.Biol. 16,5444,1996. 86.Curr.Biol. 5,477,1993. 87.J.Med.Biol.Res. 29, 895,1996. 88.NucI.Acid.Res. 24,1753,1996. 89.EMBO J. 7,3559,1988. 90.Nucl.Acid.Res. 23,4712,1995. 91.Mol.Cell.Biol. 9,5073,1989. 92.J.Cell.Blol. 115,887,1991. 93.J.Biol.Chem. 266,19867,1991. 94.Mol.Cell.Biol. 8,2737,1988; Zagyansky Y. French Patent Application FR-98-06910. 95.Eur.J.Biochem. 63,166,1994. 96.Biochem.Soc.Transac. 24,521,1996. 97.Mol.cell.Biol. 14,7984,1994. 98.J.Cell Biol. 126,1221,1994. 99.J.Neurosci. 18,1346,1996. 1 00.Gen.Compar.Endocrinol. 103,316,1996. 101.Nucl.Acid Res. 24,4078,1996. 102.Eur.J.Neurosci. 7,2249,1995. 103.Neuroscience 72,889,1996.

Important Supplement for Research—According to the Law.

According to the Law /for instance Art.52(3) of Convention of European Patent Office (EPO) or Art.1 (Austrian National Law, 1970- Administartion, charged for the International Research)l: “The provisions of paragraph 2 (as patentability of discoveries and scientific theories AS SUCH) shall exclude patentability of the subject-matter or activities reffered to in that provision only (ONLY) to the extent to which a European patent application (Austrian patent application) . . . relates to such . . . subject-matter or activities AS SUCH (it means without their practical applications)”. To see also Accords between Austrian Patent Office (EPO) and World International Patent Organisation “Gazette du PCT” 5611997 (Apperdix B): “are not excluded from research (PCT) or examination (PCT): all objects that are submitted to the research or examination according the national (Austrian or europeen) procedure”. Very clearly, as well in “Guidelines for Examination in European Patent Office”-§CIV-2.2.).

This direct stipulation is absolutely EQUVALENT (since secondary school since 12 years old, since “their” Euclide) to the stipulation, opposite to the inverse one: “If the Austrian (European) Patent Application concerns the subject-matters (as the discoveries and scientific theories) with their applications (=“not as such”), they are patentable ONLY in this case”!

It stipulates clearly according the DIRECT Law /stipulation opposite to the inverse one of Art.52(3) (EPO) or Art.1 (3) (AT)/, these subject-matters (like discoveries and scientific theories) are patentable only with their applications (!). Consequently, the claims, concerning “Theory or (EVENI) principle underlying the invention” (letter “T” according the Form of the EPO Research) (to see also “Guidelines.,” §C-2.2.) and claims, concerning their practical consequences must be considered ONLY ENSEMBLE, according the law. Consequently, I present here the (preliminary) International Classification of Patents with the mentions of the corresponding claim Groups, serving to the establishment of each element of I.C.P.

Claims 1, 2,3: C12N 7104; A61K 39/12. Claims 1, 2, 4: A61K 39/00; A61K 39/395. Claims 1, 2, 5: C12M 1/18; C12N 1/36; C120 1/70; A61K 39/12, 39/395; GOIN 33/48, 33/50. Claims 1, 2, 6, 8: A61P 37/00, 29/00, 43/00; C12P 21/00, 21/02; A81K 32/02, 38/04; C07K 1/00. Claims 1, 2, 6, 9: A61K 39/12, 39/395; C08L 101/02, 89/00. Claims 6, 10: A61K 38/46, 38/48. Claims 1, 6, 11 A61M 21/00; A61K 33/42, 31/7032. Claims 1, 6, 12: A61P 25/20; A61K 31/26.

Unity of Invention (S-03/1998 “Gazette du PCT”).

The Unity of the Invention is specially guaranteed here. The sole “sensible” claim 6 is clearly dependent “containing all characteristics of previous one (claim 1)” (S-03/1998), where moreover the “process of the signal switching, produced by interaction between gp120 and CD4 molecules” that is the important part of the “general process of AIDS development by HIV lentivirus” of claim 1, is clearly resolved “from general fundamental processes of the protein folding and recognitions” (claim 6), that is, at the same time, also, the general independent claim (general folding and recognitions) having common “particular technical elements” (Rule 13, PCT; S-03/1998,p.46.)/See for instance, example 17 (S-03/1998), where the general process of ADN sequence coding would be FIRSllV (firstlYl) resolved/.

Legends to Figures.

FIG. 1. The general schema of the Immunology /3, 4/. There are the TWO interactions between the B and T lymphocytes. The 1st interaction takes place with the help of the T cell receptors (TCR) (without CD4 receptor) and the receptors for antigen of B cells (AgR) and with the membranous immunoglobulins that are situated on the Fc receptors (FcR) of the B cells. The antigen (Ag) (⋆) (relatively large fragments of which are liberated into the solution by the macrophages “for” the 1st B-T interaction) is the bridge between the B and T cells, permitting the physical as well the localized directed chemical interactions between the lymphocytes. It is the membranous immunoglobulins that switch the signal with the flexible scorpion “tail” of the Fc fragment after the interaction with the antigen. After this interaction there is the antigen endocytosis with its consecutive appearance In the complex with the MHC-II molecules on the cell surface /5/ and the cell division in order to have the state of the corresponding phase /1/. The T4 cells have the conformational change and their CD4 receptors can make the interaction together with the TCR. During the 2nd B-T meeting there is the interaction with the TCR and CD4 receptors of the T -cells with the MHC complex presented by the B cells. This contradiction conduct to the activation, proliferation and differentiation of the B cells with the immunoglobulin production. During this phase there is the creation of the B and T cell memory. The B cell mitogens make their action only during this phase. After the thymus (TM) development (with remarkable symmetry), the T cells react by their TCR as well CD4 receptors with the macrophages (MΦ). The macrophages (the different cells of the macrophage/monocyte origin like the Langerhan cells are also included in this term) make the general unic antigne endocytosis and digestion with the antigen presentation on the surface in the complex with the MHC-II. After this interaction with the macrophages there is the T cell proliferation (symmetrical to that after the 2nd B-T Interaction) where the CD4 receptors loose the possibility to interact with the TCR. The T cell polyclonal activators act at this stage of the B-T interaction (and as one knows, they act on the T cells, justly exiting from the thymus, that clearly confirms the symmetry of this introduced schema). During the repetitive immunisations (where the well developed network of the B and T secondary memory cells exists), these B and T cells can take the majority of the antigen, processed by the macrophages from the solution ( - - - ) /8/. In this case, there is a number of receptors for antigen, that are filled by the antigens as well their fragments serve as the bridge between the B and T cells. This permits to the rigid immunoglobulins /as Immuniglobulin E (IgE) to make the cell activation without the flexible “scorpion” tail by the cross-linking of the neighboor receptors (shown In the frame). The action diapason of the IL-1, IL-2, IL-4 and IFN-γ interleukines is indicated in the general accord with work /7/. The action sites of the autoantibodies (that are the antibodies against the viral proteins: anti-gag p17, anti-gp41 (“COOH” epitope), anti-gp41 (“NH₂”-epitope)and anti-gp120 are also indicated.

FIG. 2. The best structure of the strong superpositions of the “chair” structures of the N-acetyllactosamines /βGaI(1-4)-βGIcNAc-/. All hydroxyl and substitute groups are in the equatorial positions. Note, the equatorial group on the galactose C₄ atom is the result of the torsion of its C1 ( - - - ) conformation, conducting to the flatness.

FIG. 3. The general schema of the HIV action. As a result of the 1st contamination there is the anti-env antibody creation. During the 2nd contamination there is already the productive contamination of the macrophages with the anti-env antibody help and also the contamination of the CD4 cells directly after initial contamination and the contamination by the macrophage& The apoptotic T-cell syncytium is phagocyted easier and RAPIDLY by the macrophages, diminishing the CD4 cell number by steps. At the next cycle, the new contaminated macrophages contaminate the new T4 cells making the syncytium that is again phagocyted by the macrophages. As a result, a quantity of the CD4 cells diminishes by steps without the appreciable destroyed cell traces The macrophage anergy (due to free envelope proteins) diminishes also the rate of the AIDS development. The 1st and 2nd contaminations are made by the macrophage tropic strains. The 1st contamination cannot be transmitted by parasits in difference with the 2nd one. The T-cell tropic strain (T4 cell produced!) is used for the massive contamination of the other T cells and the T-cell syncytium creation /105/. (M-macrophage, AB- anti-env antibodies).

FIG. 4. The process of the nuclear factor liberation after intracellular pH elevation with signal. A.The exchanger Na⁺/H⁺ is activated permanently. B.The intracellular pH increases for 0.1-0.3 unities. C.Consequently, the protein synthesis on the polysomes of the cytoplasm increases due to the synthesis rate increase with the pH increase (Cathepsin L synthesis included) (there is the part of CL in the form of the preprocathepsin, synthesized formely and with the dimethylated arginine ribosome attachment); D.Some of the procathepsin L excess in the cytoplasm (that is not linked with the GR peptide help) (logically without the signal peptide); E.The weak autolysis of the procathepsin L and the cathepsin L liberation (synthesized formely) from their complexes with the ribosomes, that switches the new autolysis with the CL forms, activated at the neural pH; F.The liberation with the CL help of the different proteins (NF-kB and NF-jun or bFGF included).

FIG. 5. The pathway of the vesicular cycle between the “calcisomes” (C) and the PM with the successive belts of the cortical microfilaments (consecutive) (“PKC” transporting vesicles).

1. The general process of the AIDS development by the HIV lentivirus, characterized by the following characteristics:
(1) There are the two obligatory phases of the AIDS development, coinciding with the 2 type PRINCIPAL obligatory contaminations.
(2) Only the mobile macrophages are contaminated during their movement during the 1st contamination.
(3) This movement is initiated by the cytokine secretions, which provoke: (a) the corresponding adhesion molecule appearance in the organs-targets; (b) the β-chemokine secretion serves as the chemoattractive substances for the macrophage movement, directed to the organs (encephalite origin in the case of the intensive brain contamination) wherein, Justly, these β-chemokines with their corresponding receptors coinfect the macrophages/monocytes by HIV.
(4) The relativily weak HIV concentrations can make the 1st contamination due to the patching, capping and endocytosis of many receptors, participating in the cell mobility, provoking the aggregate creations (“critic mass”), including the syndecan molecules, which make the microfilament attachment to the aggregated receptors, conducting (at the end) to the endocytosis and HIV contamination.
(5) The anti-envelope CHIV) antibodies appear later in avoiding the interactions with the viral particles of the original contamination destructed already (the basis of the 2nd contamination necessity).
(6) The 1st nonproductive HIV entry takes place with the viral replicated uninegrated DNA with the heterogeneities of DNA and the corresponding proteins, with the intracysternal A particle (IAP) presence and with the heterogenous pattern of the antibodies against the corresponding viral proteins.
(7) Such IAPs make the very weak viral pseudocontaminations of other cells due to the RNA and reverse transcriptase that they contain.
(8) The heterogeneity of the env viral proteins is generally absolutely necessary for the HIV consecutive productive contamination due to an increased complex number of the Fc fragments of the heterogenous (also) aggregated anti-env antibodies with the cellular Fc receptors that facilitates the virus penetration into the cell at the 2nd phase.
(9) The viral env protein heterogeneity often takes place at the carbohydrate attachment sites (O- and N- chains) and consequently the carbohydrate pattern of the env proteins must be especially heterogenous.
(10) The mortality acceleration at the AIDS phase takes place due to the contamination facilitation at this phase by the complement receptor (C1qR), interacting with the Ciq factor, activated by the anti-opportunist agent antibodies.
(11) The weak correspondence between the carbohydrate patterns of the Fc-receptor cell machinery (of chimpanzee) and the HIV prevent (logically) the 2nd contamination and the AIDS phase (chimpanzee “immunity”).
(12) The baby macrophages are more active than those of adult and the baby immune system can already product the antibodies very quickly after the birth, but the 2nd AIDS phase can take place only since ˜3 months of age due to the created carbohydrate pattern correspondence between the baby macrophage Fc receptor machinery and HIV virus and, generally, the 2nd contamination can take place due to the very weak transmitted dozes.
(13) The 1st macrophage contamination is made by the macrophage- tropic clones as well the 2nd contamination with also T-cell contaminations, and the T cell- tropic clones provoke the creation of syncytia that undergo the regulated and accelerated apoptosis and the phagocytosis (by macrophages) by the relatively small, almost unvisible, quantities.
(14) Principally, the vaccination must aggravate the contamination due to the antibodies, that help to the contamination, but due to the vaccination with the homogenous envelope proteins, the strong productive contamination is more problematic and these homogenous antibodies can make some decrease of the viral particle quantity (precipitation) and also some blocking of the 1st entry although in the case of the powerful 2nd HIV entry (with CD4 receptor help) there are the dangerous spontaneous reenterings.
(15) The restricted HIV-2 contaminations take place because of a weaker variability of the viral proteins during the 1st contamination and a stronger differences between the host and virus carbohydrate patterns.
(16) The discrete switching signal due to the specific interactions between the CD4 molecules and the viral envelope proteins, important for AIDS development, are determined by a more general biological molecular processes.

2. The principal characteristics of the AIDS development process /(a): there is the 1st productive contamination with the cell motility utilisation; (b) the infection increase depends on the antibodies; (c) there is the protein heterogeneity during the 1st contamination, necessary for the heterogenous antibody production, obligatory for 2nd contamination/ according claim 1 characterized In that a number of other viral FamiIles possesses them.

3. The viral exterior proteins (envelope or capsule) /Art.52(4) EPO, Art.2(2) AT (law of 1970)/ /for manufacture of medicaments pour vaccinations against viruses (like influenza A-influenza/pneumonia)/ with one (anyway with minimal possible quantity) viral neutralizing epitope and/or one (minimally possible) viral homogenous clone that must be taken for the immunization characterized in that the increase of the virus contaminations is minimal (zero) because the corresponding anti-viral antibodies could increase the cell contaminations with the Fc receptor help wherein the heterogeneity of these antibodies is, generally, obligatory for the virus entry according claims 1(8, 10, 14) and 2.

4. The substances /Art.52(4) EPO, Art.2(2) AT (law of 1970)/ (for manufacture of medicaments against encephalites), that stop the macrophage motility (as antibodies against the β-chemokine receptors), characterized in that this motility is obligatory for the contaminated macrophage entering Into brain to cause the encephalites (as those of CMV origin) according claims 1(2, 3) and 2.

5. The kit of preparations for the determination of the real titers of the different viruses, with the action, similar to the two HIV phases, according claims 1(1-5, 8 /and Art.52(4) EPO and Art.2(2) AT- law of 1970/ characterized in that for the 1st phase determination: one creates the concentration gradients of the chemokines (as β-chemokines) to switch the macrophage mobility to approach to the conditions of the 1st contamination in vivo, and for the 2nd phase contamination: one utilizes the heterogenous anti-env antibodies (similar to those in vivo) to approach to the conditions of the 2nd contamination in vivo.

6. The real characteristics of the signalling events that follow the process of the signal switching, produced by the interaction between gp120 and CD4 molecules of the “general process of AIDS development by HIV lentiviruses” of claim 1, are established from the general fundamental processes of the protein foldings and recognitions in the cells, characterized by following caracteristics:
(1) There are the chaperon specializations for each glycosylation type: N-, O- and GAG-, that determine the Universal specialities of a limited chaperon number for a protein enormous number by their carbohydrate chains, specialized due to the law of the homologous intercarbohydrate interactions.
(2) There are the two principal pathways of the protein foldings: endoptasmic reticulum→Golgi and in cytoplasm.
(3) The calnexin (chaperon of ER) is monoglycosylated and it is attached to the N-monoglycosylated proteins after the elimination of two other glucoses.
(4) The calnexin makes the complexes with the calreticulin (ER) due to their homologous O-carbohydrates of their similar structures wherein it is the calreticulin that is responsable for the complex with the BiP and grp 94 chaperons later.
(5) During the 1st “trip” in Golgi (ER→Golgi→ER→Golgi), (a) the proteolysis of the terminal N-end (or C- one) (creation of the peptide, named , Du-2T- like) must take place due to the convertases in Golgi; (b) the gag glycosylation must (can) take place.
(8) The creation of the definitive complex, moreover in ER, of the gp96/grp94 chaperon (“boat”) with the BiP, calreticulin, p50-like proteins (GAG specificity), peptidyl-prolyl isomerase (PPI) and protein-disulfide isomerase (PDI) (principally) must take place.
(7) The cytoplasmic folding of the polypeptides, yet attached to the ribosomes in cytoplasm, takes place.
(8) The Hsp7a chaperons (specialized for O-chains) and TRIC chaperons (specialized for Core 2 O-chains, the chaperon of beginning) attach to the elongating peptide near (on) the ribosome.
(9) The hsp90 chaperon serves as “the boat” (in cytoplasm) for other chaperons, 5 including the p5o chaperon, specialized for the GAG- chains (equal to cdc 37), hsp70, PPI (but without PDI), phosphatase (pp5), the CK-II and (in special case) the steroid hormone receptors).
(10) The hsp90 complex proteins (FKBP type) with the protein phosphatase and CK-II (proteins of complete “boat”) (serving to the synthesis of the signal proteins of the “PKC” vesicle transporting machinery, having the GR methylated peptide at the C- or N- ends of preproprotein) all go to the nucleus (with these signal proteins) to attach to the preribosomes in nucleus (nucleole) to leave it at the end of G1 (or between signals of “G0”).
(11) The hsp90 complex proteins (“boat” type CyP) do not go to the nucleus and serve to the cytoplasm folding of the “purely” cytoplasrnic proteins without the GR peptides and without the “trip” into the nucleus.
(12) The Erp61 (grp58) protein is not the chaperon (of PDI family), but it is clearly the vesicular Pl-PLC-α, that is well active on “PKC” vesicles, although only after the limited proteolysis, and it is responsable for the phosphatidyl inositol-4,5-biphosphate (PIP₂) molecule hydrolysis on the “PKC” transporting vesicles during their spontaneous stopping (like with oxygene cutting) during their functioning at G1 cycle phase or “G0”, leading to, at the end, irreversibility of their definite stopping (famous apoptosis!) due to the stock exhaustion of these PIP₂, absolutely necessary for the “PKC” transport vesicle movement (IA,§6; Parts VII, XI), where such irreversibility (apoptosis) could be also due to the stock exhaustion (of proteins, attached with GR peptides on cytoplasmic ribosomes) of the proteins of the “PKC” transporting vesicle machinery.
(13) Particularly, the cytoplasmic steroid receptors “wait” the hormone in the hsp9o “boat” complex (type FKBP) wherein the steroid signal provokes the limited proteolysis (by CL) of the proreceptor and its liberation (with pH increase and protein phosphatase activation).
(14) Generally, the active state with the tension, necessary for the energy stocking during the protein functionings, is made with the two PPI enzymes, that make the two coupled transitions (prolyl trans→cys of two prolyl neighbouring residues with the weak difference of the energies between these positions) wherein the return is impossible because the one sole transition is uneffective for the return and two transitions (at the same time!) are improbable.
(15) In the case of the ER-Golgi pathway, there is the protein foldings with the help of the proteolysed propeptide (from N- or C- ends), attached to the macromolecules and hidden by the two homologous interacting carbohydrate chains, wherein the folding is accomplished with the distortion mechanism by the tension with the PPI and PDI help wherein with the artificial peptide loss in vivo (“scarpie” form) there is the pathology.
(16) In the case of the cytoplasmic protein foldings, the molecules of the “PKC” transporting vesicle machinery, having the GR peptides and making the “trip” into nucleus there is, firstly, the new chaperon assembling (based on the “boat” hsp90/type FKBP/) with the preproteins, having the GR peptide,in the nucleus (nucleole) in constructing the preproribosomes, that after the activation (at G1 end) go into cytoplasm, wherein there is (with signal /G1 beginning/) the folding of the new proteins of the machinery with the PPI (FK-506 sensible) help, where the GR prepeptide plays a role, during folding, for the attachment to the ribosomes and later, In the “ready” form, for the “adress” of the destination with the liberation of the 2 interacting (firstly intramolecular interaction) carbohydrate chains, for already “definitive”, intermolecular interactions, with the 2 (also “intra”intreracting before) glycochains of the target, correspondingly.
(17) In the case of the “pure” cytoplasmic proteins (representing many enzymes) (without “trip” into nucleus), the folding is necessary for the activity (as enzymatic for the tension energy stocking) wherein the propeptide (N- or C-) is necessary for the folding (tension energy) with the PPI (CyP-like) help and where the peptide is not important for the enzyme activity although its association must “abate” definitively the homologous carbohydrate chain dissociation to interact specifically with the chains of the target (adress) of the cytoplasmic proteins.
(18) The proteins (including enzymes) that can be folded with such chaperons must obligatory have their primary structure with the two nearby prolines, corresponding “Du-2T” proteolysable peptides in the proprotein (including the C-end possibility) and corresponding amino acid cluster, permitting the posttranslational modifications with the couple (at least) of the carbohydrate chains, possessing their intramolecular homologous Interactions, hiding moreover the “Du-2T” peptide.
(19) The proteins, submitted to this general fundamental process of the protein foldings possess the characteristics for the general recognitions (“adresses”): the couple of their carbohydrate chains being in the intramolecular intercarbohydrate homologous liaison can recognize justly the homologous couple of the (also in intramolecular interactions) carbohydrate homologous chains and provoke the signal beginning with the 2 inter molecular (already) intercarbohydrate hydrogen homologous liaisons, that is “abated” definitively by the corresponding “Du-2T” peptide dissociations.
(20) Particularly, the real characteristics of the signalling events in the process of the AIDS development are established from this (established here) general processes of the protein foldings and recognitions: the couples of the interacting carbohydrate chains of the CD4 molecules (Ochains near the hinge regions) and C2 and V3 regions of gp120 can recognize each other with the switching of the dissociations of these INTRAmolecular bonds for the establishment of the INTERmolecular intercarbohydrate hydrogen homologous liaisons between CD4 and gp120 chains already, that is “abated” definitively by the dissociation of the corresponding “Du-2T”-like proteins wherein as a result of the strong conformational change, the other intramolecular liaisons are broken, leading to the agglomerate creations with the membrane melting due to the intercarbohydrate dehydration.

7. The little proteins (peptides) Du-2T like (“Du-2T”) of the synthesized, by ER→Golgi, proteins, including all membranous receptors (as CD4 or gpi2o) of claims 1(16) ,2 and 6 characterized in that they are obtained. from N- or C- proprotein ends after the limited proteolysis, they are hidden at the protein surface by the 2 carbohydrate chains with the homologous intercarbohydrate interactions, they are situated near the site of the 2 prolines and S—S bond(s) (source of the irreversible folding with tension) and its dissociation changes the state of the general structure of these proteins profoundly and discretely in liberating the carbohydrate chains for the intermolecular interactions (for formation of the complex aggregate as in the case of the membranous receptors or/and activation of other molecules) like these real little proteins (peptides) for IgG (and also for Fc receptors and receptors for antigen), characterized in that they are situated in the CH2 region and they dissociate after the hapten. (antigen) interaction with the active sites because of the consecutive dissociation of the Intramolecular coupled interactions of the covering homologous chains in liberating these imrnunoglobulin chains from already intermolecular interactions: with N-carbohydrate chains of the complement (C1q) (in activating it) or with the carbohydrates of other receptors: Fc and for antigen and with other carbohydrate chains of plasrna membrane (PM); like the little real proteins (peptfdes) (although exceptionally special) Du-2T- like for the molecules MHC class I, characterized in that they are found in “the active site”, created by the al and a2 domains of the MHC α-chain with the several prolines of these subdomains wherein “the dissociation” of this peptide (Du-2T -like) in the active site of the TRC provokes the strong change of the general structure of the MHC-I αchain, where the sole N-chain carbohydrate (without pair at invariable site) of this subunit can already interact with the corresponding carbohydrate chain (of the invariable site) of the TCR a-chain (also without pair) to switch the “Du-2T” dissociation from the MHC α3 domain with the 2 covering carbohydrate chains, the dissociation, from TCR, of its “Du-2”, (also there is the presence of the prolines, S—S and two symmetric N-carbohydrate chains) (very exceptional presence of the charged amino acids in the intramembranous domains of all components of TCR facilitates the confornational changes due to instability) and the mutual intermolecular interactions of the O- and N- chains of MHC with those of TCR and the CD8 molecules (having own “Du-2T”) and between the carbohydrate chains of the same PM (during the complete signal); and like the functional GPI-anchored proteins (so called priones), synthesized by ER→Golgi (in complex aggregate of the classical complete signal) that make the self-aggregation in pathology (diseases as Mad Cow or Jacob-Creutzfeldt) (this time, without signal already, but like during the signal!), due to the interactions with the forms of these proteins where the “Du-2T” is dissociated already and the carbohydrate chains (hiding) are free for already homologous intermolecular aggregation, facilitating already the dissociation (facilitated also generally!) the dissociation of the “Du-2T” of other molecules.

8. The large number of the different little proteins (peptides) (Du-2T-like=“Du-2T”) according claims 1,2,6 and 8 for the manufacture of the medicaments /Art.52(4) EPO, Art.2(2) AT (Law 1970)/ against (1) the undesirable process of the complement activation; (2) the cell lysis; (3) the action of the particular membranous receptors; (4) the action of the parasites as the viruses and also bacteria, protozoans, mashrooms; (5) the creation of the dangerous priones (so called aggregated “scarpie” form) during the diseases as Mad Cow or Creutzfeldt-Jacob; (6) the protein aggregation in solutions (In blood included) characterized in that the simple Introduction of these “Du-2T” proteins must stop the corresponding undesirable processes in preventing the dissociation of their native analogues where such dissociation would provoke the harmful signalling Including the pathological diseases /(1)-(5)/ or the intermolecular glycoprotein aggregation in solution (6).

9. The well charged affine molecules (as antibodies) against the distinct HIV surface molecules (or those of other viruses and other parasites like bacteria or mashrooms or against distinct active sites of the surface antibodies, receptors for antigens (B-cells) and/or TCR producing the harmful anti-HIV-env antibodies (or similar harmful antibodies against the other viruses), the harmful auto-antibodies (as anti-HIV-gag or those in rheumatic diseases) or the harmful allergic antibodies, characterized in that these strong, localizedprecisely, specifically charges perturb the intercarbohydrate homologous hydrogen liaisons of the signallings of HIV and other viruses as well the functioning of the cell, producing these harmful antibodies for manufacture of medicaments /Art.52(4) EPO, Art.2(2) AT (Law 1970)/ according claims 1, 2 and 6 (19, 20).

10. The process of the functioning cycle of the cytoplasmic ribosomes for the folding (FKBP type of the proteins for the transporting “PKC” vesicle machinery) and the synthesis of all proteins at all ribosomes according claim 6 is characterized in that (1) During already the signal (in G1 or “G0” or the stocking signal at the G1 end), there is the constitution of the preproribosornes (nucleus, nucleole) from the proteins (with GR peptides), synthesized and folded on the active ribosomes (cytoplasm) (nuclear proteins as nucleolin or fibrillarin or ribosomal proteins as L5 or of the machinery of the transporting “PKC” vesicles /to be stocked/ or the steroid receptors that attach to the rRNA in nucleus /Inucleole/); and these preproribosomes are activated (1st step!) by the special signal with the nuclear (serine or cysteine) proteinases (where the proteolysis of the N-part of the nucleolin, attached to the chromatin, Is necessary for the pre-rRNA transcription) and they go to the cytoplasm where the 3′ mRNA part serves as the guide for the cytoskeletal localisation; (2) These proribosomes (cytoplasm) are already activated definitively with the cathepsin L (CL) help that cuts (at GR peptides) the particular ribosomal proteins as L5 and the nuclear proteins as the nucleolin and fibrillarin (serving as fusible ) and (CL) liberates also the stockedproteins of the “PKC” transporting vesicle machinery (necessary to start again the machinery of the proteins of the “PKC” transporting vesicle cycles without their synthesis) wherein these active ribosomes make the new synthesis and foldings with the help of the corresponding chaperons (FKBP type), attached yet from the nucleus, where, naturally, all ribosomes for all proteins, have the same cycles but without “traveling” proteins with the GR peptides where the same effective process of the protein synthesis of all proteins at the proribosomes (in reality, obtained from the cytoplasm) in vitro (attached for instance on the artificial surfaces) must be made with such CL activation: proribosomesribosomes.

11. The manufactured medicaments /Art.52(4) EPO, Art.2(2) AT law; Accord WIPO-AT/ against the state of the clinical death and coma as phosphatidylinositol-4,5-biphosphate or its derivatives including the lysoderivatives (with easier integration in PM with transport to the interior PM layer) and, like GTP-γS (less hydrolysable substratum also for vesicle transport) characterized in that all these substances help to avoid the process of the irreversible apoptosis of the cells of the brain and heart (original reason of the state of the clinical death and coma) according claims 1 and 6.

12. The manufactured medicaments (hypnotics) /Art.52(4) EPO, Art.2(2) AT and Accord WIPO-AT) for the partial Inhibition of the cycle of the “PKC” and synaptic (“PKC”-Iike) vesicles (like very deluted cyanate) for the sleep process are characterized in that they cut partially the cyclic system of the neurons of the superior brain (determining the cycle, establishing the conscience), functioning by the chaotic permanent cycles of the synaptic (“PKC”-like) transport vesicles according claim 6(12).
